(19) United States
(12) Patent Application Publication
(10) Pub. No.: US 2007/0111947 A1
(43) Pub. Date: May 17, 2007

(54) Fibrin targeted therapeutics

(76) Inventors: Thomas J. McMurry, Winchester, MA (US); Andrew Kolodziej, Winchester, MA (US); Alan P. Carpenter JR., Carlisle, MA (US); Simon Jones, Somerville, MA (US); Philip Graham, Carlisle, MA (US); Richard Looby, Reading, MA (US); Shrikumar A. Nair, Lexington, MA (US); Xifang Wang, Malden, MA (US); Kirsten Overoye-Chan, Chelmsford, MA (US); John A. Barrett, Groton, MA (US)

Correspondence Address: FISH & RICHARDSON P.C., PO BOX 1022, MINNEAPOLIS, MN, 55440-1022, US

(21) Appl. No.: 11/581,677

(22) Filed: Oct. 16, 2006

Related U.S. Application Data
(60) Provisional application No. 60/800,152, filed on May 12, 2006.
(60) Provisional application No. 60/726,632, filed on Oct. 14, 2005.

(51) Int. Cl. A61K 38/10; C07K 7/08

(52) U.S. Cl. 514/15; 530/328

(57) ABSTRACT

Fibrin targeted therapeutic agents for the treatment of thromboembolism, infection, and cancer are provided.

### CROSS-REFERENCE TO RELATED APPLICATIONS

This application claims priority under 35 U.S.C. § 119(e) to U.S. Provisional Application No. 60/800,152, filed May 12, 2006, and to U.S. Provisional Application No. 60/726,632, filed Oct. 14, 2005. Both applications are incorporated by reference in their entirety herein.

### TECHNICAL FIELD

This invention provides hybrid molecules that include both a therapeutic bioactive moiety and a fibrin-targeting moiety. More particularly, the invention provides hybrid molecules in which a bioactive moiety is conjugated optionally through a linker to a fibrin-targeting moiety. A bioactive moiety can be specific for the treatment of thromboembolism, infection, and cancer.

### BACKGROUND

Current methods for treating thromboembolism, such as arteriothrombosis and venous thrombosis, is currently limited. Examples of arteriothrombotic conditions include coronary thrombosis (e.g., myocardial infarction resulting from plaque rupture), unstable angina, pulmonary embolism, atrial fibrillation, stroke, thrombosis in AJV fistula, and in-stent thrombosis. Venous thrombosis includes deep vein thrombosis and pulmonary embolism as an ancillary complication. Treatment of these conditions with antithrombotics such as anticoagulants, antiplatelets, thrombolytics, and fibrinolytics can lead to many unwanted side-effects. For example, intravenous injection of thrombolytics that act systemically rather than at specific clot locations can cause complications due to bleeding. Accordingly, there is a need for antithrombotics that can localize at the site of clots.

Current human cancer therapies, while significantly improved from earlier therapies, also have limitations. For example, the non-specific toxicity of most antitumor agents against normal cells leads to complications in medical treatment. Numerous attempts have been made to minimize this non-specificity by targeting the drugs at or near the tumor cells through hybridization of the drugs with a biotargeting moiety. This strategy can increase agent exposure while reducing systemic toxicity. Early efforts have been directed toward either utilizing specific cell surface receptors that have been found to be upregulated on tumor cells, or taking advantage of enhanced metabolism in tumor cells for increased agent uptake. Despite these advances, there remains a need for new cancer therapies.

Current therapies for infections also are limited in efficacy. Infective endocarditis (IE) therapy, for example, involves prolonged treatment with bactericidal antibiotics (typically 4-6 weeks), and attempts to balance systemic toxicity and efficacy. A characteristic infectious lesion (i.e., the vegetation) is an amorphous mass of platelets and fibrin in which abundant microorganisms are enmeshed. Adherence of the microorganisms to fibrin is a crucial step in the progress of an infection such as IE. Thus, there is a continued need for improved methods for treating infection.

### SUMMARY

The invention is based on fibrin targeted therapeutic agents, for the treatment of thromboembolism, infection, and cancer, which can localize a drug at the site of pathology with increased efficacy and reduced systemic toxicity. Fibrin is an abundant extracellular matrix (ECM) protein found in many diseases, including thromboembolism and cancer. The fibrin targeted therapeutic agents are conjugates of a fibrin-targeting moiety and a bioactive moiety such as an antithrombotic (e.g., anticoagulant or antiplatelet), antitumor agent or chemotherapeutic agent (e.g., cytotoxin), antibiotic, or radiopharmaceutical (e.g., radionuclide (including chelated radionuclides)). The fibrin-targeting moiety and the bioactive moiety can be covalently joined, optionally, through a linking group. The targeted therapeutics of the invention can provide enhanced efficacy and reduced systemic toxicity relative to a corresponding non-targeted bioactive moiety. The present fibrin-targeting hybrid molecules can exhibit modulated biodistribution upon administration to a mammal as compared to a free drug control (i.e., without the fibrin-targeting moiety), which can result in an improved therapeutic index. For example, hybrid molecules that include a thrombin inhibitor (e.g., melagatran) bound to a fibrin-targeting moiety may achieve effective exposure at thrombi while keeping systemic concentrations at a lower level.

In one aspect, the invention features a hybrid molecule of the general formula [D]ᵢ-[L]ⱼ-[F]ₖ, wherein [D] includes a bioactive moiety for treating thromboembolism, infection, and cancer, [L] includes a linker moiety, and [F] includes a fibrin-targeting moiety, wherein i and k are independently integers between 1 and 20, and j is an integer from 0 to 20. In some embodiments, i is 1 to 5 (e.g., 1, 2, 3, 4, or 5). In some embodiments, k is 1 to 5 (e.g., 1, 2, 3, 4, or 5). In other embodiments, j is 1 to 5 (e.g., 1, 2, 3, 4, or 5). The fibrin-targeting moiety can be a peptide, a peptidomimetic, or a small molecule.

In another aspect, the invention features a hybrid molecule of the general formula [D]ᵢ-[L]ⱼ-[F]ₖ-[L′]ⱼ-[Z]ₘ, wherein [D] includes a bioactive moiety for treating thromboembolism, infection, and cancer, [L] and [L′] independently include a linker moiety, [F] includes a fibrin-targeting moiety, and [Z] includes a modifying moiety wherein i, and k are independently integers between 1 and 20, j is independently an integer from 0 to 20, and m is independently an integer from 0 to 20. The fibrin-targeting moiety can be a peptide, a peptidomimetic, or a small molecule. The modifying moiety may be used to modulate the solubility, physiological stability, metabolic stability, biodistribution, or other physical properties of the hybrid molecule.

The peptide can have the amino acid sequence P*-Y*-X₁*-L*, wherein P* is a proline or a non-natural derivative thereof; Y* is a tyrosine or a non-natural derivative thereof; X₁* is G or D or a non-natural derivative of G or D; L* is a leucine or a non-natural derivative thereof. X₁* can be G or D and L* can be leucine. P* can be proline or 4-hydroxyproline, and Y* can be tyrosine or a non-natural derivative of tyrosine substituted at the 3 position with a moiety such as F, Cl, Br, I, and NO₂.

The peptide can have the amino acid sequence X₁-X₂-C-P*-Y*-X₃-L-C-X₄-X₅-X₆, wherein P* is a proline or a non-natural derivative thereof; Y* is a tyrosine or a non-natural derivative thereof; X₁ is selected from the group consisting of W, Y, F, S, Bip, Hx, Dpr, Cy, Gu, Ad, Hfe, 3-Pal, 4-Pal, DopaMe₂, nTyr, dW, dF, F(3/4*), and Y(3*), wherein F(3/4*) is a phenylalanine substituted at either the 3 or the 4 position with a moiety selected from the group consisting of CH₃, CF₃, NH₂, CH₂NH₂, CN, F, Cl, Br, I, Et, and OMe, and wherein Y(3*) is a tyrosine substituted at the 3 position with a moiety selected from the group consisting of F, Cl, Br, I, and NO₂; X₂ is selected from the group consisting of E, H, dE, S, H(Bzl), 2-Pal, Dpr, and Th; X₃ is selected from the group consisting of G and D; X₄ is selected from the group consisting of H, F, Y, and W; X₅ is selected from the group consisting of I, L, V, N, Bpa, Bal, Hfe, Nle, Tle, Nval, Phg, Cha, Taz, Fua, Th, 4-Pal, and F(3/4*), wherein F(3/4*) is a phenylalanine substituted at either the 3 or the 4 position with a moiety selected from the group consisting of CF₃, Et, iPr, and OMe; X₆ is selected from the group consisting of N, Q, I, L, and V, or X₆ is not present. P* can be proline or 4-hydroxyproline, and Y* can be a tyrosine or a non-natural derivative of tyrosine substituted at the 3 position with a moiety selected from the group consisting of F, Cl, Br, I, and NO₂. In some embodiments, X₄ can be H.

The peptide can have the amino acid sequence C-P*-Y*-X₁-L-C, wherein X₁ is G or D, P* is proline or its non-natural derivative 4-hydroxyproline; Y* is tyrosine or a non-natural derivative of tyrosine substituted at the 3 position with a moiety selected from the group consisting of F, Cl, Br, I, and NO₂.

The peptide can have the amino acid sequence C-D-Y-Y-G-T-C-X₁₀, wherein X₁₀ is selected from the group consisting of n(decyl)G, n(4-PhBu)G, MeL, Bpa, Bip, Me-Bip, F(4*), F(3-Me), F(3,4-difluoro), Amh, Hfe, Y(3,5-di-iodo), Pff, 1Nal, d1Nal, and MeL, wherein F(4*) is a phenylalanine or phenylalanine substituted at the 4 position with a moiety selected from the group consisting of Et, CF₃, I, and iPr.

The peptide can have the amino acid sequence C-D-Y-Y-G-T-C-X₁₀-X₁₁ wherein X₁₀ is selected from the group consisting of n(decyl)G, n(4-PhBu)G, MeL, Bpa, Bip, Me-Bip, F(4*), F(3-Me), F(3,4-difluoro), Amh, Hfe, Y(3,5-di-iodo), Pff, 1Nal, d1Nal, and MeL, wherein F(4*) is a phenylalanine or phenylalanine substituted at the 4 position with a moiety selected from the group consisting of Et, CF₃, I, and iPr, and X₁₁ is D, dD, βD, Inp, Nip, Me-D, dc, Cop, or Cmp. The peptide also can have a formula of structure 1-11.

The bioactive moiety can be an antithrombotic. An antithrombotic can be, for example, an anticoagulant, antiplatelet, thrombolytic, or fibrinolytic. An anticoagulant moiety can affect, e.g., inhibit, directly or indirectly, the action of enzymes, polypeptides, and cofactors involved in thrombin generation (e.g., the thrombin coagulation cascade), including thrombin itself, or it may block the action of an inhibitor of fibrinolysis. Alternatively, an antiplatelet moiety may affect, e.g., inhibit, directly or indirectly, an aspect of platelet function, including platelet adhesion to a wound site, platelet activation (e.g., shape change or filapodia extension), platelet aggregation, platelet secretion of prothrombotic or platelet activating factors, and recruitment and activation of monocytes.

The bioactive moiety can be an anticoagulant moiety such as a thrombin inhibitor (e.g., melagatran), Factor Xa inhibitor, tissue factor inhibitor, Factor VIIa inhibitor, Factor IXa inhibitor, Factor Va inhibitor, Factor XIa inhibitor, Factor XIIa inhibitor, TAFIα inhibitor, α2-antiplasmin inhibitor, PAI-1 inhibitor, PAI-2 inhibitor, PAI-3 inhibitor, prothrombinase inhibitor, tick anticoagulation peptide, protein C, warfarin, heparin, lepirudin, aspirin, ticlopidine, clopidogrel, tirofiban, and eptifibatide.

The bioactive moiety can be an antiplatelet moiety such as GPIIb/IIIa receptor inhibitor, ADP receptor (e.g., P2Y₁, and P2Y₁₂) inhibitor, thrombin receptor (e.g., PAR-1 and PAR-4) inhibitor, CD40 inhibitor, CD40L (CD40 ligand) inhibitor, Gas6 inhibitor, Gas6 receptor axl inhibitor, Gas 6 receptor inhibitor Sky, Gas6 receptor Mer inhibitor, P-selectin inhibitor, P-selectin receptor PSGL-1 inhibitor, thromboxane inhibitor, synthetase inhibitor, fibrinogen receptor antagonist, prostacyclin mimetic, phosphodiesterase inhibitor, RANTES inhibitor, phosphoinositide-3-kinase (PI(3)K) isoform β inhibitor, phosphoinositide-3-kinase (PI(3)K) isoform γ inhibitor, eptifibatide, tirofiban, ticlopidine, and clopidogrel.

Thrombolytics can also be used as bioactive moieties and include tissue plasminogen activator (natural or recombinant), urokinase, plasminogen activators (e.g., vampire bat plasminogen activator), streptokinase, staphylokinase, prourokinase, anisolated streptokinase plasminogen activator complex (ASPAC), and animal salivary gland plasminogen activator.

The bioactive moiety can also include fibrinolytics such as copperhead snake fibrolase.

The bioactive moiety also can be any compound with general antithrombotic activity that reduces thrombus formation such as hyaluronic acid or dextran.

In some embodiments, the bioactive moiety can be an antibiotic such as a β-lactam antibiotic, sulfonamide, quinolone, trimethoprim, aminoglycoside, tetracycline, macrolide, probenecid, chloramphenicol, or glycopeptide. The β-lactam antibiotic can be a penicillin, cephalosporin, or carbapenem. The penicillin can be penicillin, ampicillin, nafcillin, oxacillin, or methicillin. The cephalosporin can be cepahlothin, cefamandole, ceftaxidime, deacetylcephalothin, cephaloridine, cefixime, or latamoxef. The macrolide can be erythromycin, clarithromycin, or azithromycin. The aminoglycoside can be kanamycin or streptomycin. The glycopeptide can be vancomycin.

The bioactive moiety can be a chemotherapeutic or antitumor agent. For example, a bioactive moiety can be a cytotoxic agent such as an alkylating agent (nitrogen mustards, nitrosoureas, or triazenes), antimetabolite (e.g., folic acid analogs, pyrimidine analogs, or purine analogs), natural product (e.g., alkaloid, ellipticine, hydroxymethylacylfulvene, epipodophyllotoxin, enzyme, biological response modifier, or hormone), antibiotic (e.g., actinomycin D, daunorubicin, doxorubicin, or idarubicin), platinum coordination complex, or antagonist (e.g., antiestrogens or antiandrogens).

A bioactive moiety can include a radiopharmaceutical, such as a chelated radionuclide. The radionuclide can include Iodine-131, Yttrium-90, Lutetium-177. Copper-67, Rhenium-186, Rhenium-188, Bismuth-212, Bismuth-213, and Astatine-211. The chelating group can include DTPA and DOTA.

The linker can be cleavable, for example, by a proteolytic enzyme such as a protease selected from the group consisting of matrix metalloproteases, blood coagulation factors, neutrophil elastase, prostate specific antigens, and plasminogen activators.

The modifying moiety can be a metal chelate, charged moiety, or other organic or inorganic chemical group.

In another aspect, the invention features a pharmaceutical composition for the treatment of thromboembolism, infection, and cancer. The composition includes a hybrid molecule as described and at least one pharmaceutically acceptable ingredient. The pharmaceutically acceptable ingredient can be a solubilizing agent, buffer, vehicle, preservative, local anesthetic, flavoring, coloring, stabilizer, or excipient.

In yet another aspect, the invention provides a method for treating thromboembolism. The method includes administering an amount of a composition effective to treat thromboembolism, e.g. preventing clot formation, reducing the growth rate of a clot, reducing the size of a clot or thrombus in any dimension, or maintaining or improving the patency of a blood vessel. For example, the size of a clot can be reduced by 5% or more in any dimension (e.g., 10%, 20%, 30%, 40%, 45%, 50%, 55%, 60%, 65%, 70%, 75%, 80%, 85%, 90%, 92%, 95% or more).

The composition includes a hybrid molecule, wherein the bioactive moiety is an anticoagulant such as a thrombin inhibitor (e.g., melagatran), Factor Xa inhibitor, tissue factor inhibitor, Factor VIIa inhibitor, Factor IXa inhibitor, Factor Va inhibitor, Factor XIa inhibitor, Factor XIIa inhibitor, TAFIα inhibitor, α2-antiplasmin inhibitor, PAI-1 inhibitor, PAI-2 inhibitor, PAI-3 inhibitor, prothrombinase inhibitor, tick anticoagulation peptide, protein C, warfarin, heparin, lepirudin, aspirin, ticlopidine, clopidogrel, tirofiban, and eptifibatide.

In other cases, the bioactive moiety can be an antiplatelet moiety, such as GPIIb/IIIa receptor inhibitor, ADP receptor (e.g., P2Y₁, and P2Y₁₂) inhibitor, thrombin receptor (e.g., PAR-1 and PAR-4) inhibitor, CD40 inhibitor, CD40L (CD40 ligand) inhibitor, Gas6 inhibitor, Gas6 receptor axl inhibitor, Gas6 receptor inhibitor Sky, Gas6 receptor Mer inhibitor, P-selectin inhibitor, P-selectin receptor PSGL-1 inhibitor, thromboxane inhibitor, synthetase inhibitor, fibrinogen receptor antagonist, prostacyclin mimetic, phosphodiesterase inhibitor, RANTES inhibitor, phosphoinositide-3-kinase (PI(3)K) isoform β inhibitor, phosphoinositide-3-kinase (PI(3)K) isoform γ inhibitor, eptifibatide, tirofiban, ticlopidine, and clopidogrel.

Furthermore, the bioactive moiety can be a thrombolytic agent such as tissue plasminogen activator (natural or recombinant), urokinase, plasminogen activators (e.g., vampire bat plasminogen activator), streptokinase, staphylokinase, prourokinase, anisolated streptokinase plasminogen activator complex (ASPAC), and animal salivary gland plasminogen activator.

In another embodiment, the bioactive moiety can be a fibrinolytic such as copperhead snake fibrolase.

The hybrid molecule may be given alone or in combination with anticoagulant, antiplatelet, thrombolytic, or fibrinolytic drugs.

In yet another aspect, the invention provides a method for treatment in which the administration of the hybrid molecule alone or in combination with thrombolytics is used to reduce re-occlusion or re-thrombosis following initial clot degradation and restoration of blood flow.

An improved therapeutic index can be achieved with these hybrid molecules. For example, by conjugating the present fibrin-targeting peptides to inhibitors of thrombin, Factor Xa, or inhibitors of platelet activation, aggregation, and secretion, improved efficacy and/or safety is observed.

The invention also features methods for treating infection in a mammal. The method includes administering an amount of a composition to the mammal effective to treat the infection, e.g., to reduce the number of infectious microorganisms. The composition includes a hybrid molecule, wherein the bioactive moiety is an antibiotic. The antibiotic may be selected from a β-lactam antibiotic, sulfonamide, quinolone, trimethoprim, aminoglycoside, tetracycline, macrolide, probenecid, chloramphenicol, or glycopeptide. The β-lactam antibiotic can be a penicillin, cephalosporin, or carbapenem. The penicillin can be penicillin, ampicillin, nafcillin, oxacillin, or methicillin. The cephalosporin can be cepahlothin, cefamandole, ceftaxidime, deacetylcephalothin, cephaloridine, cefixime, or latamoxef. The macrolide can be erythromycin, clarithromycin, or azithromycin. The aminoglycoside can be kanamycin or streptomycin. The glycopeptide can be vancomycin. The symptoms of the infection may or may not be reduced.

In another aspect, the invention features a method of treating cancer in a mammal. The method includes administering an amount of a composition effective to treat the cancer e.g., to reduce the number of cancerous cells or the size of a tumor. The composition includes a hybrid molecule, wherein the bioactive moiety is an antitumor or chemotherapeutic agent such as a cytotoxic agent. Examples of cytotoxic agents include an alkylating agent (nitrogen mustards, nitrosoureas, or triazenes), antimetabolite (e.g., folic acid analogs, pyrimidine analogs, or purine analogs), natural product (e.g., alkaloid, ellipticine, hydroxymethylacylfulvene, epipodophyllotoxin, enzyme, biological response modifier, or hormone), antibiotic (e.g., actinomycin D, daunorubicin, doxorubicin, or idarubicin), platinum coordination complex, or antagonist (e.g., antiestrogens or antiandrogens). Symptoms of the cancer may or may not be reduced.

Compositions of any of the above methods can be administered orally, intraperitoneal, or by intravenous or subcutaneous injection.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 shows non-natural amino acids or amino acid derivatives that may be constituents of the peptide targeting group of the invention.

FIG. 2 exemplifies an increased time to occlusion of approximately 4-fold utilizing Hybrid Molecule 3 (a fibrin-targeted thrombin inhibitor containing melagatran as the bioactive moiety) as described in Example 5 (“binder”). The “Non-binder” control is the equivalent molecule with the exception that the cysteine at the C-terminal end of the peptide is switched to the d-amino acid form instead of the 1-amino acid form. A comparison of Hybrid Molecule 3's results as compared to an equivalent dose of melagatran alone or the Non-binder control is shown.

FIG. 3 exemplifies a decrease in clot weight of approximately 2-fold utilizing Hybrid Molecule 3 as compared to an equivalent dose of melagatran or Non-binder control.

FIG. 4 exemplifies a higher level of Hybrid Molecule 3 compared to an equivalent dose of Non-binder control bound to clot (5-fold) or to clot plus damaged artery (2-fold).

FIG. 5 shows the effect of Compound 12 (see Example 17) on two tumor cell lines, human umbilical vein endothelial cells, and vascular smooth muscle cells.

### DETAILED DESCRIPTION

Definitions

A “hybrid molecule” refers to a molecule comprising a fibrin-targeting moiety covalently linked, optionally through a linking moiety, to a bioactive moiety. The hybrid molecule may also have a modifying moiety covalently linked, optionally through a linking moiety.

A “bioactive moiety” refers to agents for the treatment or prevention of disease, in particular, for treating thromboembolism, infections and cancer.

A “drug” refers to any chemical agent used in the treatment, cure, prevention, or diagnosis of disease (Pharmacology-Drug Actions and Reactions, Ruth R. Levine, Parthenon Publishing, 6th edition). By this definition, a “drug” can be, but is not limited to, a small molecule or a polypeptide.

A “fibrin-targeting moiety” refers to a molecule or portion of a molecule that specifically binds to fibrin or to a soluble or insoluble fragment of fibrin. A fibrin fragment may have a structure or characteristic exhibited by fibrin. The term fibrin-targeting moiety as used herein does not encompass recombinant, chimeric, humanized, monoclonal, or polyclonal antibodies or antibody fragments, including single chains that are specific for fibrin or a soluble or insoluble fragment of fibrin.

An “inhibitor” refers to any chemical agent that reduces the activity of a protein (e.g., an enzyme) either through active-site direction, e.g. competitive inhibitor and suicide inhibitor, exo-site direction, e.g. noncompetitive inhibitor and uncompetitive inhibitor, or an overlapping mechanism of action.

The term “specific binding affinity” as used herein, refers to the capacity of a molecule to bind a particular biological component to a greater degree than other components. The specific binding affinity of a molecule for a target can be expressed in terms of the equilibrium dissociation constant “Kd.”

A “modifying moiety” refers to agents used to adjust the physical, physiological and/or biological properties of the resulting hybrid molecule. In addition, a “modifying moiety” may be an imaging agent to provide visualization.

The term “treating thromboembolism” refers to preventing or reducing the rate of growth of a clot or thrombus accretion in any dimension. Thromboembolism is a general term describing both thrombosis and its main complication which is embolisation. The term thromboembolism, as used herein, is used interchangeably with thrombosis and thromboembolic disease. The treatment of thromboembolism may also include reducing the size of a clot or thrombus in any dimension. The size of the clot or thrombus may be reduced by an amount of 5% or more (e.g., 10%, 20%, 30%,40%, 45%, 50%, 55%, 60%, 65%, 70%, 75%, 80%, 85%, 90%, 92%, 95% or more). The patency of the blood vessel containing the thrombus or clot may be maintained or increased, e.g., as assessed by angiography, including X-ray angiography, MR angiography, ultrasound, or doppler flow. By maintaining or prolonging vessel patency, a longer time window is provided in which intervention may be efficacious. The treatment of thromboembolism may also be prophylactic, used for example in combination with percutaneous coronary or peripheral artery intervention, or elective surgery, e.g. athroplasty.

The term “treating infection” refers to reducing the number of infectious microorganisms (e.g., bacteria). The number may be reduced by an amount of 5% or more (e.g., 10%, 20%,30%, 40%, 45%, 50%, 55%, 60%, 65%, 70%, 75%, 80%, 85%, 90%, 92%, 95% or more). Symptoms of the infection may or may not be reduced.

The term “treating cancer” refers to reducing the number of cancerous cells or reducing the size of a tumor. The number of cancerous cells or the size of the tumor may be reduced by an amount of 5% or more (e.g., 10%, 20%, 30%, 40%, 45%, 50%, 55%, 60%, 65%, 70%, 75%, 80%, 85%, 90%, 92%, 95% or more). Symptoms of the cancer may or may not be reduced.

The term “non-natural amino acid” or “non-natural” refers to any derivative of a natural amino acid including D forms, and β and γ amino acid derivatives. It is noted that certain amino acids, e.g., hydroxyproline, that are classified as a non-natural amino acid herein, may be found in nature within a certain organism or a particular protein.

The terms “peptide” and “polypeptide” are used interchangeably herein, and refer to a chain of amino acids that ranges from two to about 75 amino acids in length, or any value therebetween. For example, a peptide can be about 2 to about 50, about 2 to about 25, about 2 to about 15, about 2 to about 12, about 4 to about 15, about 4 to about 12, about 6 to about 12, about 5 to about 20, about 5 to about 25, or about 5 to about 12 amino acids.

Hybrid Molecules

In general, methods of treating thromboembolism, infection, and cancer using fibrin targeted hybrid molecules, which are conjugates of a fibrin-targeting moiety and a bioactive moiety, are provided. The bioactive moiety can include, for example, anticoagulant moieties, antiplatelet moieties, thrombolytics, fibrinolytics, antibiotics, radiopharmaceuticals (e.g., chelated radionuclides), antitumor agents, or combinations of such bioactive moieties. The fibrin-targeting and bioactive moieties can be covalently joined, optionally through a linking group.

The therapeutic hybrid molecules include a bioactive moiety (D), which can be, but is not limited to, drugs known to have beneficial therapeutic effects against thromboembolism, infection, and cancer, and a fibrin-targeting moiety (F), which has affinity for insoluble polymeric fibrin. The two moieties can be covalently joined together, optionally through a linker moiety (L). In some embodiments, the hybrid molecules have the following general formula:
[D]ⱼ-[L]ⱼ-[F]ₖ
where i and k are integers ranging from 1 to 20. For example, k and i can independently range from 1 to 5. J can be an integer from 0 to 20, e.g., from 1 to 5.

More broadly, the invention features a hybrid molecule of the general formula:
[D]ᵢ-[L]ⱼ-[F]ₖ-[L′]ⱼ-[Z]ₘ
wherein [D] includes a bioactive moiety for treating thromboembolism, infection, and cancer, [L] and [L′] independently include a linker moiety, [F] includes a fibrin-targeting moiety, and [Z] includes a modifying moiety, wherein i, and k are independently integers between 1 and 20, j is independently an integer from 0 to 20, and m is independently an integer from 0 to 20. The fibrin-targeting moiety can be a peptide, a peptidomimetic, or a small molecule. The modifying moiety may be used to modulate the solubility, physiological stability, biological activity or other physical properties of the hybrid molecule.
Fibrin-targeting Moieties

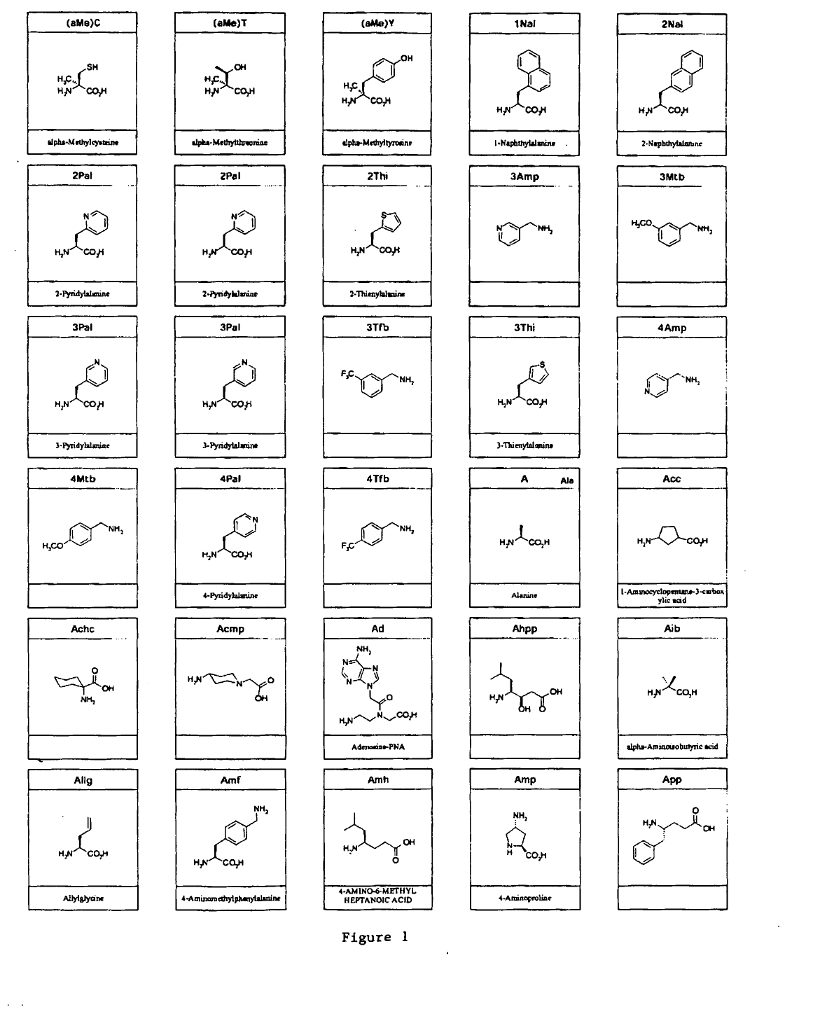

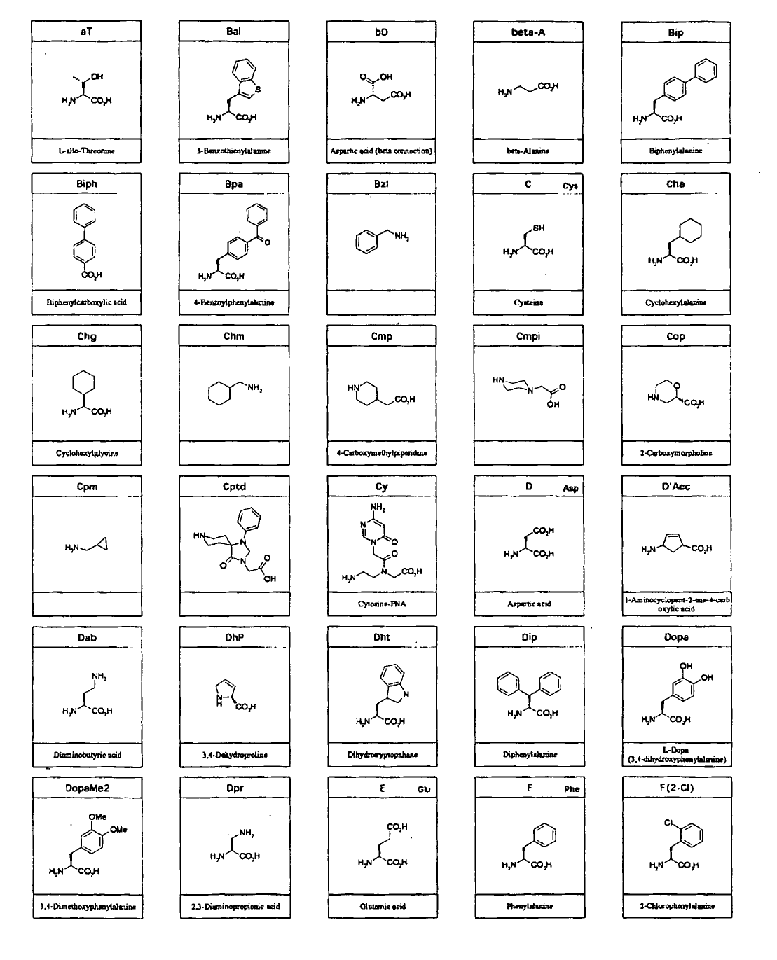

Suitable fibrin-targeting moieties can be a small molecule, a peptide, or a peptidomimetic. Fibrin-targeting peptides can include a great variety of amino acids, including natural and non-natural amino acids. Amino acids with many different protecting groups appropriate for immediate use in the solid phase synthesis of peptides are commercially available. In addition to the twenty most common naturally occurring amino acids, the following non-natural amino acids or amino acid derivatives may be constituents of the peptide targeting group of the invention (common abbreviations are in parentheses), see FIG. 1: β-Alanine (β-Ala), γ-Aminobutyric Acid (GABA), 2-Aminobutyric Acid (2-Abu), α,β-Dehydro-2-aminobutyric Acid (Δ-Abu), 1-Aminocyclopropane-l-carboxylic Acid (ACPC), Aminoisobutyric Acid (Aib), 2-Amino-thiazoline-4-carboxylic Acid, 5-Aminovaleric Acid (5-Ava), 6-Aminohexanoic Acid (6-Ahx), 8-Aminooctanoic Acid (8-Aoc), 11 -Aminoundecanoic Acid (11-Aun), 12-Aminododecanoic Acid (12-Ado), 2-Aminobenzoic Acid (2-Abz), 3-Aminobenzoic Acid (3-Abz), 4-Aminobenzoic Acid (4-Abz), 4-Amino-3-hydroxy-6-methylheptanoic Acid (Statine, Sta), Aminooxyacetic Acid (Aoa), 2-Aminotetraline-2-carboxylic Acid (Atc), 4-Amino-5-cyclohexyl-3-hydroxypentanoic Acid (ACHPA), para-Aminophenylalanine (4-NH2-Phe), Biphenylalanine (Bip), para-Bromophenylalanine (4-Br-Phe), ortho-Chlorophenylalanine (2-Cl-Phe), meta-Chlorophenylalanine (3-Cl-Phe), para-Chlorophenylalanine (4-Cl-Phe), meta-Chlorotyrosine (3-Cl-Tyr), para-Benzoylphenylalanine (Bpa), tert-Butylglycine (Tle), Cyclohexylalanine (Cha), Cyclohexylglycine (Chg), 2,3-Diaminopropionic Acid (Dpr), 2,4-Diaminobutyric Acid (Dbu), 3,4-Dichlorophenylalanine (3,4-C12-Phe), 3,4-Diflurorphenylalanine (3,4-F2-Phe), 3,5-Diiodotyrosine (3,5-12-Tyr), ortho-Fluorophenylalanine (2-F-Phe), meta-Fluorophenylalanine (3-F-Phe), para-Fluorophenylalanine (4-F-Phe), meta-fluorotyrosine (3-F-Tyr), Homoserine (Hse), Homophenylalanine (Hfe), Homotyrosine (Htyr), 5-Hydroxytryptophan (5-OH-Trp), Hydroxyproline (Hyp), para-Iodophenylalanine (4-1-Phe), 3-Iodotyrosine (3-I-Tyr), Indoline-2-carboxylic Acid (Idc), Isonipecotic Acid (Inp), meta-methyltyrosine (3-Me-Tyr), 1-Naphthylalanine (1-Nal), 2 Naphthylalanine (2-Nal), para-Nitrophenylalanine (4-NO2-Phe), 3-Nitrotyrosine (3-NO2-Tyr), Norleucine (Nle), Norvaline (Nva), Ornithine (Om), ortho-Phosphotyrosine (H2PO3-Tyr), Octahydroindole-2-carboxylic Acid (Oic), Penicillamine (Pen), Pentafluorophenylalanine (F5-Phe), Phenylglycine (Phg), Pipecolic Acid (Pip), Propargylglycine (Pra), Pyroglutamic Acid (pGlu), Sarcosine (Sar), Tetrahydroisoquinoline-3-carboxylic Acid (Tic), and Thiazolidine-4-carboxylic Acid (Thioproline, Th). Stereochemistry of amino acids may be designated by preceding the name or abbreviation with the designation “D” or “d” or “L” or “1” as appropriate. Additionally, αN-alkylated amino acids may be employed, as well as amino acids having amine-containing side chains (such as Lys and Orn) in which the amine has been acylated or alkylated. For example, the peptide can be a fibrin binding peptide disclosed in WO 01/09188 or WO 03/011115. For example, the peptide can have the amino acid sequence CXYYGTC or CPYXLC, where X is G or D.

Peptides of the invention can include the general formula P*-Y*-X₁*-L*, wherein P* is a proline or a non-natural derivative of proline, Y* is a tyrosine or a non-natural derivative thereof, X₁* is glycine or aspartic acid, or a non-natural derivative of glycine or aspartic acid, and L* is leucine or a non-natural derivative thereof. In some embodiments, at least one of P*, Y*, X₁*, or L* is a non-natural derivative of the respective amino acid. For example, X₁* can be glycine or aspartic acid, L* can be leucine, and at least one of P* or Y* can be a non-natural derivative, such as hydroxyproline or a tyrosine substituted at the 3 position with F, Cl, Br, I, or NO₂.

A peptide of the invention also can include the general formula X₁-X₂-C-P*-Y* -X₃-L-C-X₄-X₅-X₆, wherein P* is a proline or a non-natural derivative thereof; Y* is a tyrosine or a non-natural derivative thereof; X₁ is W, Y, F, S, Bip, Hx, Dpr, Cy, Gu, Ad, Hfe, 3-Pal, 4-Pal, DopaMe₂, nTyr, dW, dF, F(3/4*), or Y(3*). F(3/4*) can be a phenylalanine substituted at either the 3 or the 4 position with a moiety such as CH₃, CF₃, NH₂, CH₂NH₂, CN, F, Cl, Br, I, Et, or OMe. Y(3*) can be a tyrosine substituted at the 3 position with a moiety such as F, Cl, Br, I, and NO₂. X₂ can be E, H, dE, S, H(Bzl), 2-Pal, Dpr, or Th; X₃ can be G or D; X₄ can be H, F, Y, or W; X₅ can be I, L, V, N, Bpa, Bal, Hfe, Nle, Tle, Nval, Phg, Cha, Taz, Fua, Th, 4-Pal, or F(3/4*), wherein F(3/4*) is a phenylalanine substituted at either the 3 or the 4 position with a moiety such as CF₃, Et, iPr, or OMe; X₆ can be N, Q, I, L, or V, or not present. In some embodiments, at least one of X₁, X₂, X₅, P*, and Y* is a non-natural derivative of an amino acid. For example, P* can be proline and Y* can be a non-natural derivative of tyrosine substituted at the 3 position with a moiety such as F, Cl, Br, I, or NO₂ Alternatively, P* can be a non-natural derivative of proline such as 4-hydroxyproline and Y* can be tyrosine. We have found in some cases that when X₄ is H improved plasma stability for the therapeutic hybrid molecules is found. Such peptides can form a disulfide bond under non-reducing conditions.

Another example of a peptide that can bind fibrin includes the general formula C-P*-Y*-X₁-L-C, wherein X₁ is G or D, P* is proline or its non-natural derivative 4-hydroxyproline; Y* is tyrosine or a non-natural derivative of tyrosine substituted at the 3 position with a moiety such as F, Cl, Br, I, or NO₂. At least one of P* or Y* can be a non-natural derivative of the respective amino acid. For example, the peptide can have the following sequences: W-dE-C-P(4-OH)-Y(3-Cl)-G-L-C-W-I-Q, Y-dE-C-P(4-OH)-Y(3-Cl)-G-L-C-Y-I-Q, Y-dE-C-P(4-OH)-Y(3-Cl)-G-L-C-W-I-Q, W-dE-C-P(4-OH)-Y(3-Cl)-G-L-C-Y-I-Q, W-dE-C-P(4-OH)-Y(3-Cl)-D-L-C-W-I-Q, Y-dE-C-P(4-OH)-Y(3-CI)-D-L-C-Y-I-Q, Y-dE-C-P(4-OH)-Y(3-Cl)-D-L-C-W-I-Q, W-dE-C-P(4-OH)-Y(3-Cl)-D-L-C-Y-I-Q, F(4-OMe)-H-C-P(4-OH)-Y(3-Cl)-D-L-C-H-I-L, Y-H-C-P(4-OH)-Y(3-Cl)-G-L-C-W-I-Q, W-dE-C-P-Y(3-Cl)-G-L-C-W-I-Q, W-dE-C-P(4-OH)-Y-G-L-C-W-I-Q, or F-H-C-P-(4-OH)-Y(3-Cl)-D-L-C-H-I-L. Such peptides can form disulfide bonds under non-reducing conditions. Each peptide can have a K_{d} for fibrin or a fibrin fragment of ≦10 μM.

A peptide also can have the general formula C-D-Y-Y-G-T-C-X₁₀, wherein X₁₀ is n(decyl)G, n(4-PhBu)G, MeL, Bpa, Bip, Me-Bip, F(4*), F(3-Me), F(3,4-difluoro), Amh, Hfe, Y(3,5-di-iodo), Pff, 1Nal, d1Nal, or MeL, wherein F(4*) is a phenylalanine or a phenylalanine substituted at the 4 position with a moiety such as Et, CF₃, I, or iPr. In some embodiments, a peptide can include additional residues, X₁, P*, and/or X₁₁, to provide the general formula: C-D-Y-Y-G-T-C-X₁₀-X₁₁or X-P*-C-D-Y-Y-G-T-C-X₁₀-X₁₁, wherein X₁ is any natural or non-natural amino acid, P* is proline or a non-natural derivative thereof, and X₁₁ is D, dD, βD, Inp, Nip, Me-D, Cop, or Cmp. For example, a peptide can have the sequence of L-P-C-D-Y-Y-G-T-C-n(Decyl)G-dD, L-P-C-D-Y-Y-G-T-C-n(Decyl)G-D, L-P-C-D-Y-Y-G-T-C-Bip-D, L-P-C-D-Y-Y-G-T-C-Bip-dD, L-P-C-D-Y-Y-G-T-C-MeL-Inp, L-P-C-D-Y-Y-G-T-C-MeL-Cmp, or L-P-C-D-Y-Y-G-T-C-MeBip-D.

Peptides having the formula X₁-P*-C-D-Y-Y-G-T-C-X₁₀-X₁₁ can be synthesized according to standard synthesis methods, such as those disclosed in WO 01/09188, WO 01/08712, or WO 03/011115, and assayed for affinity to the DD(E) fragment of fibrin, which contains subunits of 55 kD (Fragment E) and 190 kD (Fragment DD). The DD(E) fragment can be biotinylated and immobilized via avidin to a solid substrate (e.g., a multi-well plate). Peptides can be incubated with the immobilized DD(E) fragment in a suitable buffer and binding detected using known methodologies. See, for example, WO 01/09188 and WO 03/011115. Alternatively, the peptide may be labeled on the N or C terminus with a fluorescein moiety and binding of the labeled peptide to DD(E) may be measured by fluorescence polarization. Binding of non-labeled peptides to DD(E) may be assessed by displacement of the fluorescein peptides from DD(E) in competition assays (see WO 01/09188)

Preferred fibrin-targeting moieties possess a high binding affinity for fibrin or a soluble or insoluble fragment thereof (i.e., K_{d} ranging from about 10⁻⁸ M to about 10⁻⁴ M) and favorable pharmacological properties. Fibrin binding peptides can fall within two classes, which are exemplified by structures 1-11 in Scheme 1. In class 1, binding elements are contained within the seven amino acid residue disulfide loop. The consensus residues are the central Y-Y-G-T segment. The conformation of the disulfide bridged ring structure may affect binding affinity. The exocyclic residues can contribute further to binding affinity. This class of peptides are exemplified by structures 1-5.

In class 2, binding elements are contained within the six amino acid residue disulfide loop. The consensus residues are the central P*-Y*-X-L segment, where X is D or G, and P* and Y* are as described previously. The conformation of the disulfide bridged ring structure may affect binding affinity. The exocyclic residues contribute further to binding affinity. This class of peptides are exemplified by structures 6-11.

Modifying the peptide can be used to vary the plasma half-life and physiochemical properties of the hybrid molecules to optimize the dose, ease of formulation, route of administration and biological activity for a given therapeutic use. Scheme 1. Structure of Fibrin binding Peptides 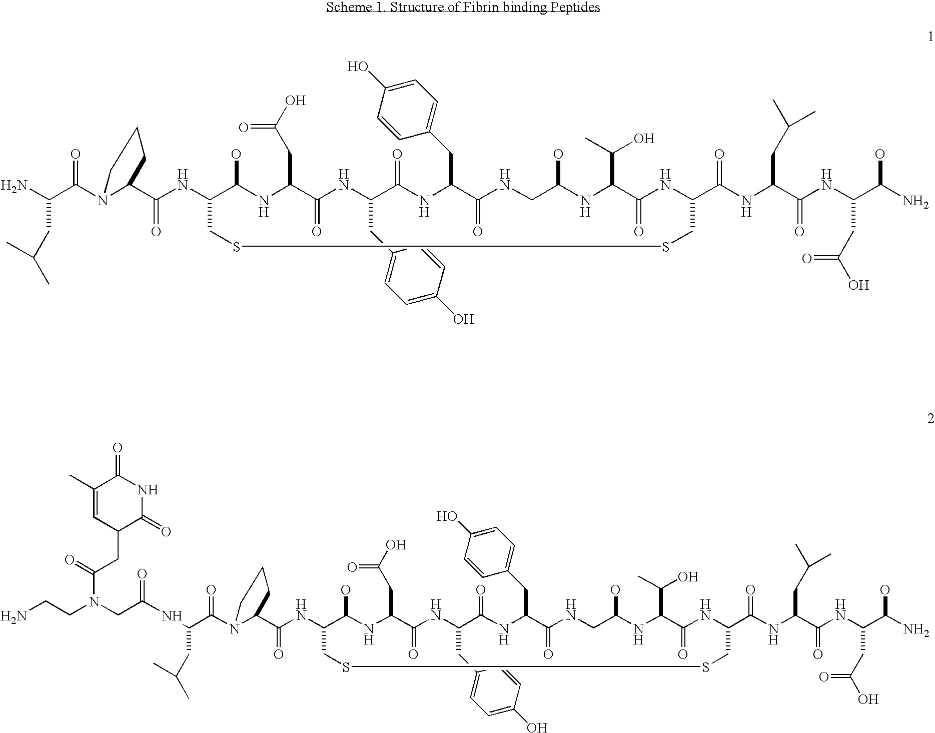 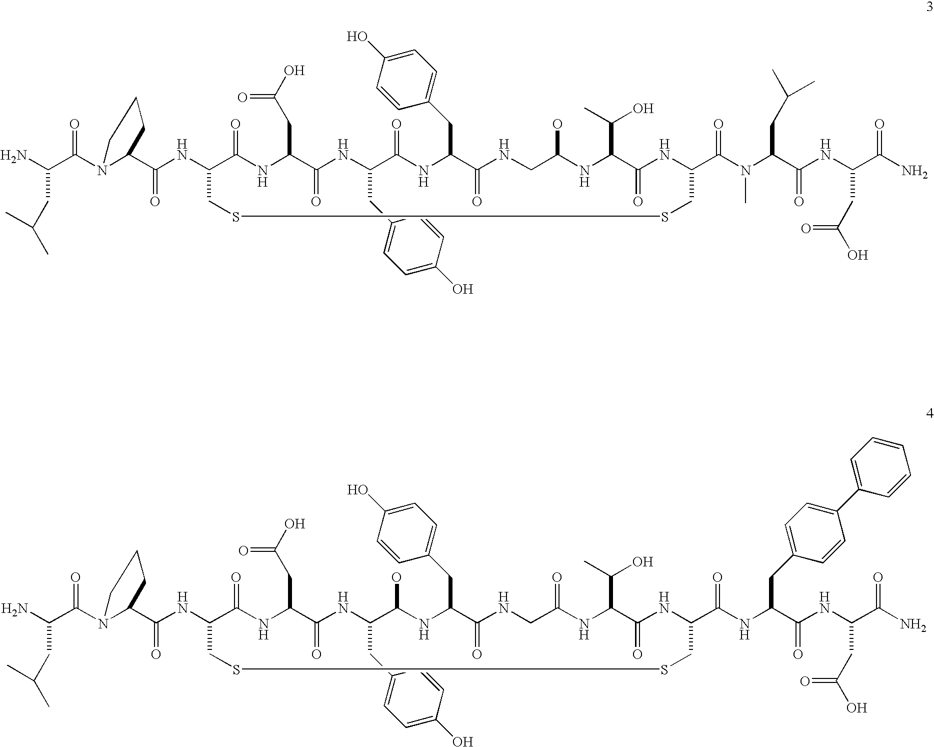 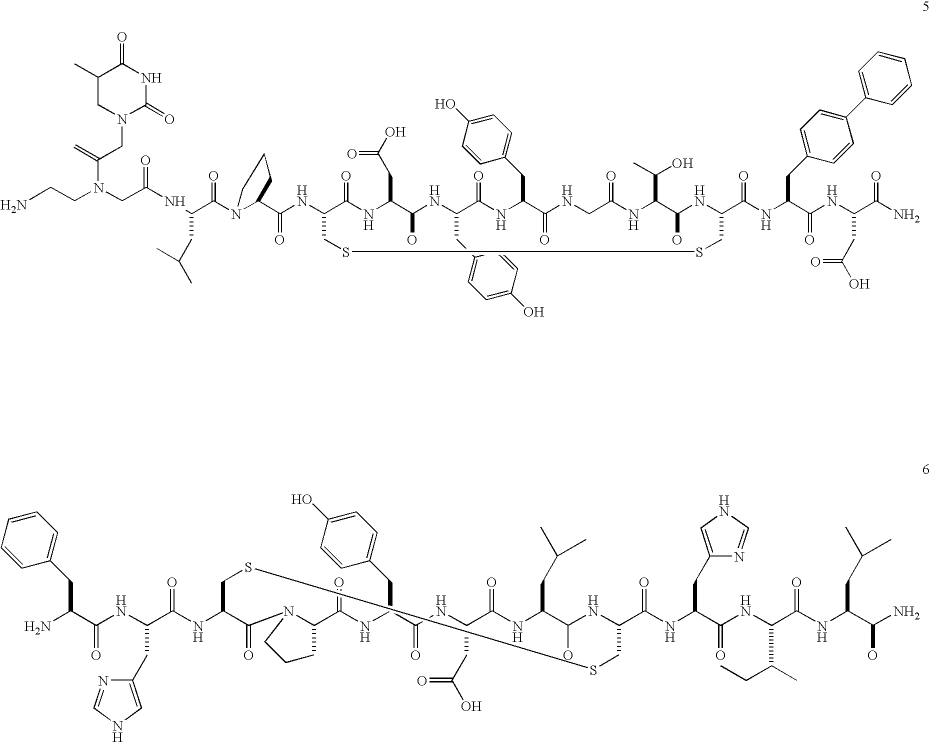 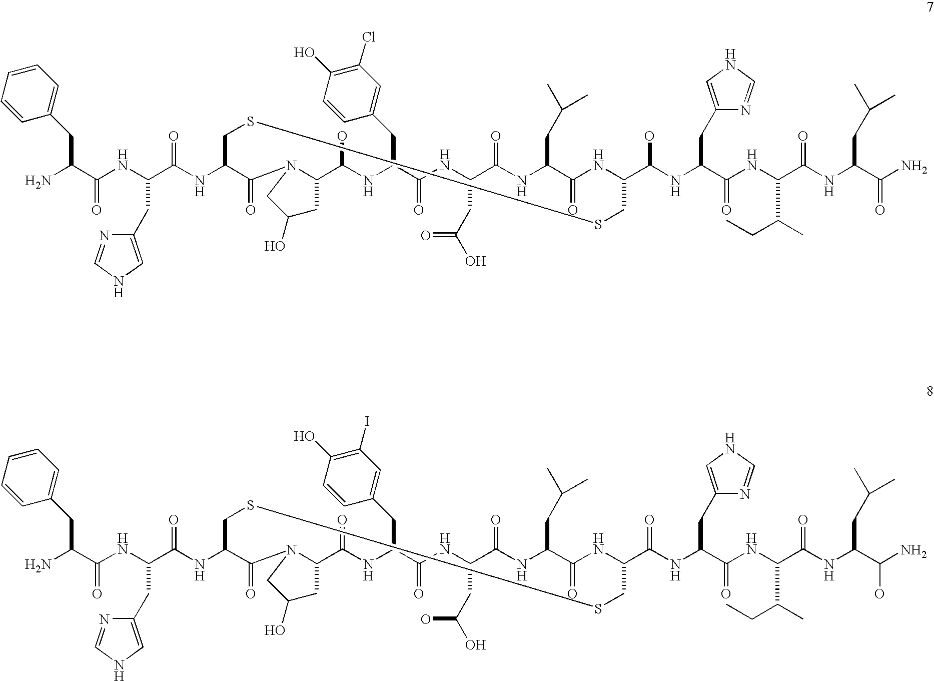 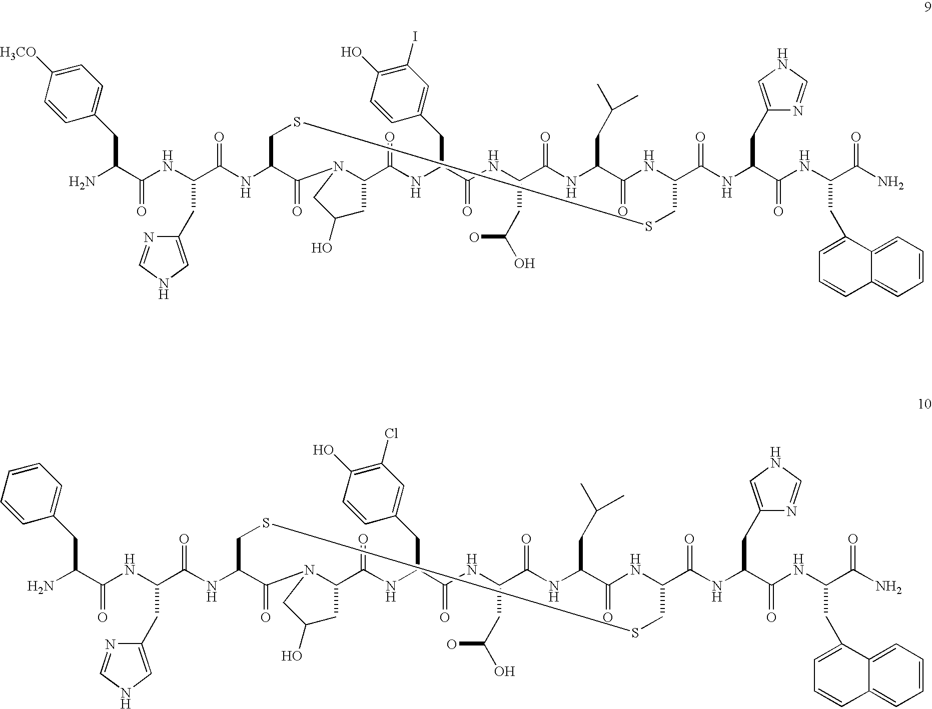 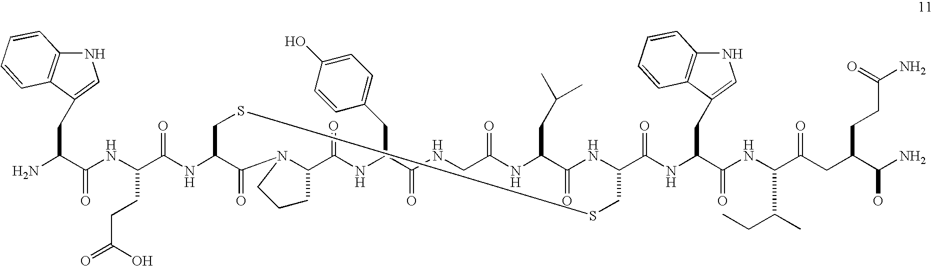
Linker Moieties

Bioactive moiety [D] and fibrin-targeting moiety [F] can be directly bonded to each other. Alternatively, bioactive moiety [D] and the fibrin-targeting moiety [F] can be joined through a linker [L]. The linker can be peptidic or non-peptidic in nature. The linker can be an all-carbon chain, or can contain heteroatoms such as, e.g., oxygen, nitrogen, sulfur, and phosphorus. The linker can contain a PEG (polyether) region. The linker can be a linear or branched chain, or can include structural elements such as phenyl ring(s), non-aromatic carbocyclic or heterocyclic ring(s), double or triple bond(s), and the like. Linkers may be substituted with halo, pseudohalo, alkyl, alkenyl, alkynyl, alkoxy, cycloalkyl, aryl, amino, hydroxy, carbonyl, alkoxycarbonyl, and hydroxycarbonyl groups. The linker moieties can include multiple functional groups, which can be conjugated to one or more fibrin-targeting and bioactive moieties.

In some instances, it is desirable to cleave the bioactive moiety from the fibrin-targeting moiety at the lesion site. In this case, a cleavable linker may be employed. The linker may be cleaved, for example, chemically (e.g., by hydrolysis, reduction, or oxidation reaction) or by enzyme catalyzed processes. Cleavable linkers can be short peptide sequences specifically recognized and processed by proteolytic enzymes and include, for example, proteases selectively upregulated at sites of pathological lesions, such as certain matrix metalloproteases (MMPs), blood coagulation factors, neutrophil elastase, prostate specific antigen, and plasminogen activators (PAs). Processing of such linkers results in local release of the bioactive moiety [D]. In this case, the bioactive moiety [D] can be more therapeutically active than the hybrid molecule alone. This “prodrug” approach has become an established strategy in the pharmaceutical industry to achieve favorable distribution properties for certain drugs. Exemplary linkers, some of which may be cleavable, are presented in Scheme 2.

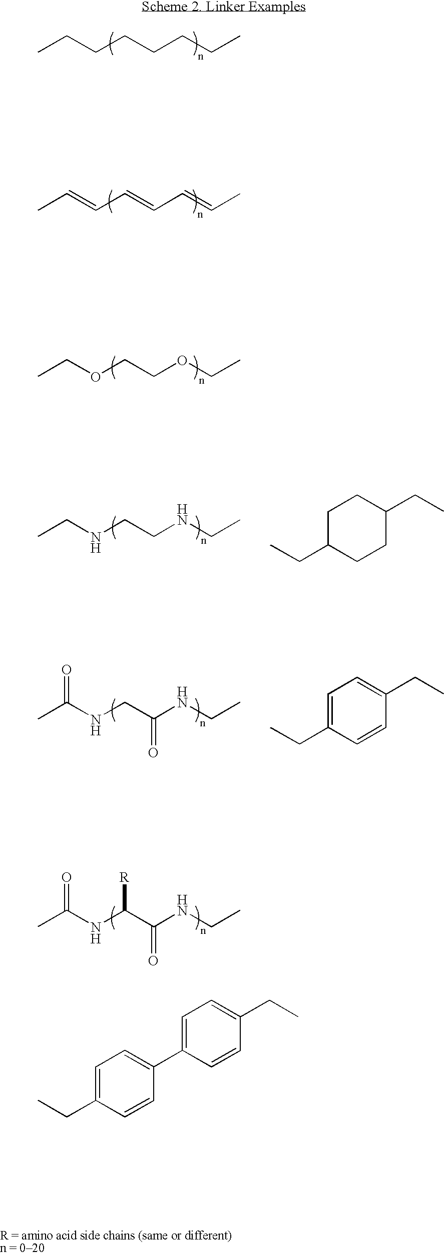

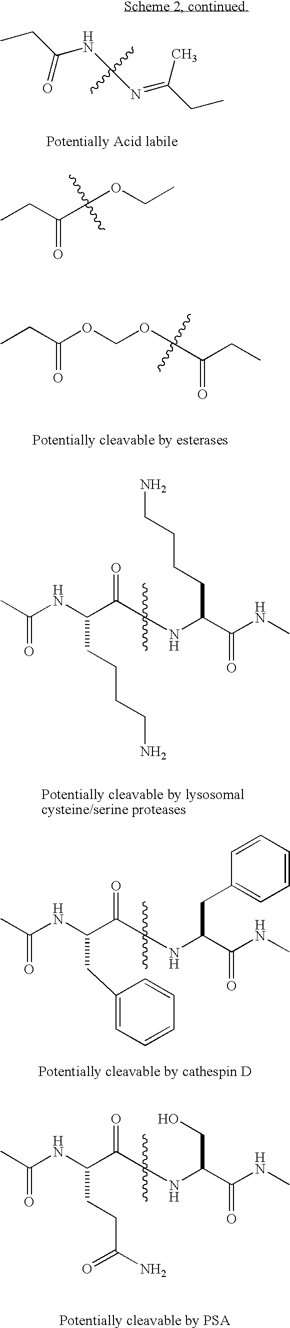

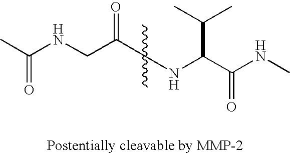

The linker may comprise multiple functional groups for attachment of one or more fibrin-targeting and bioactive moieties. For example, a linker that includes a carbonyl with a leaving group LG (for example, a carboxylic acid or an activated ester) and three or more protected amines can be reacted with a peptide amine to create a molecule with three or more terminal amines. The following carbonyl-based linker reagents may be appropriate for introducing three or more amine functional groups: 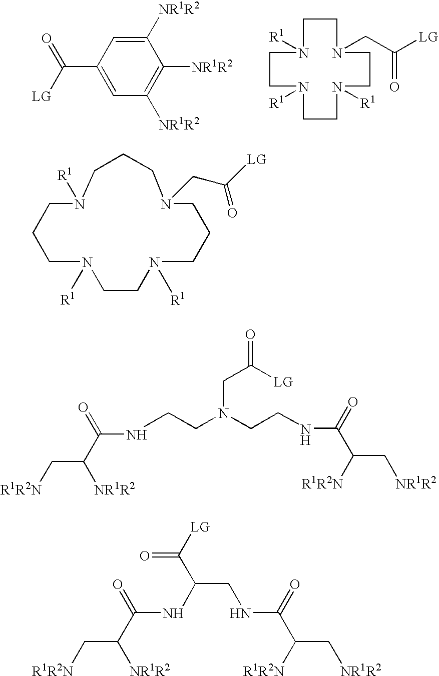
wherein LG is a leaving group (e.g., -OH, activated ester such as pentafluorophenol (Pfp), N-hydroxysuccinimide (NHS), N-hydroxysulfosuccinimide sodium salt (NHSS), 2-thioxothiazolidin-1yl, or hydroxybenzotriazole (HBT) and R¹ and R² are preferably independently hydrogen or a chemical protecting group (e.g., Boc, Fmoc, CBZ, t-butyl, benzyl, or allyl).

The amine functional group at the N-terminus of a peptide may be converted to an N-terminus carboxylate functional group by reaction with a cyclic acid anhydride thereby producing a modified peptide with an N-terminal carboxylate functional group:

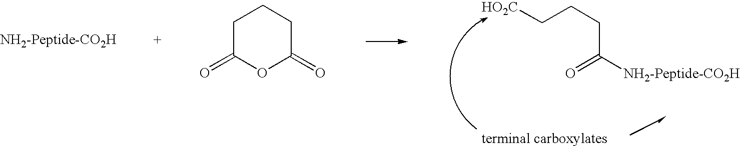

Examples of other reagents that can be used to convert an N-terminal amine to a carboxylate functional group include: 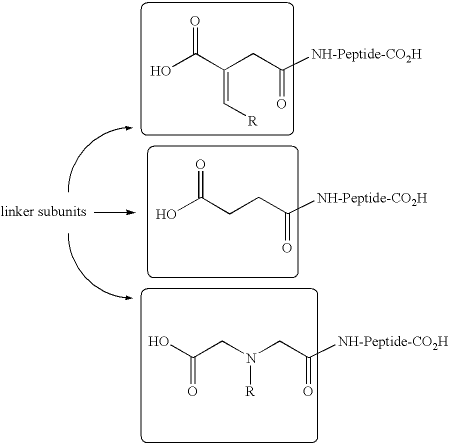
wherein R is any aliphatic or aromatic group.

When following an amide bond construction strategy in which a peptide molecule is terminated with two carboxylates, the following linker reagents may be appropriate to introduce three or more amine functional groups: 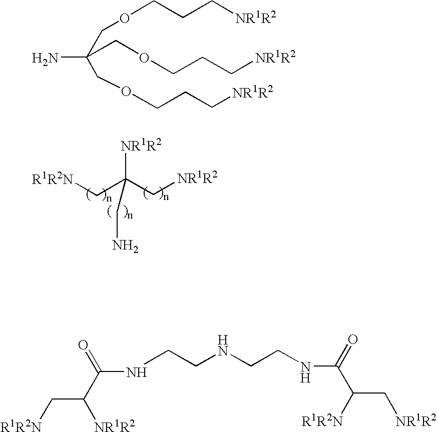
wherein R¹ and R² are independently hydrogen or a chemical protecting group such as OS, Boc, Fmoc, CBZ, t-butyl, benzyl, or allyl. Linker strategies that involve formation of amide bonds are useful because they typically are compatible with the protecting groups on the peptide. The linker can be covalently attached to the fibrin-targeting peptide(s) by formation of other bond types of reactions (e.g., nucleophilic displacement, or thiourea formation).

Conjugation to a protein or polypeptide bioactive moiety can be achieved by standard chemical techniques including the formation of amide, ester, disulfide, and thioether bonds. For example, a fibrin binding peptide can be covalently linked either directly, or through a linker, to a protein or polypeptide by forming an amide bond between the fibrin binding peptide or the linker and one or more lysine residues, e.g., on the surface of a protein. Surface lysine residues are usually distant from the enzyme's catalytic site. Therefore, the tethered moieties may not interfere with the enzyme's catalytic activity. Multiple ligation can be achieved in a single step. The proportion of the fibrin-targeting peptide relative to the protein drug can be controlled by adjusting stoichiometry of the ligation chemistry. Multiple ligation is particularly useful in the case of a moderately strong fibrin binding ligand because higher binding affinity can be realized through the so called “avidity” effect. In particular, a coupling agent or an activated ester can be used to achieve the amide bond formation between the lysine and the fibrin binding moiety or the linker. Alternatively, the fibrin-targeting peptide can be incorporated into the hybrid molecule using recombinant DNA technology.

Bioactive Moieties

The bioactive moiety can be directed towards improving therapies currently used for the treatment of thromboembolism, infection, and cancer. As compared with cell surface receptors, fibrin exists in much higher concentrations and is prevalent during various stages of these disease states. Therapeutics currently used for such diseases display serious side effects derived from freely circulating drugs. By connecting the therapeutic moiety to a fibrin-targeting group, efficacy of these therapeutics can be enhanced, and their toxicity can be reduced.

Antithrombotics

The fibrin-targeting moiety of the current invention can be used to improve the therapeutic index of currently used antithrombotics, including thrombolytics, fibrinolytics, antiplatelets, and anticoagulants. For example, an improved therapeutic index can be achieved by conjugating the present fibrin-targeting peptides to thrombolytics. A thrombolytic can affect, directly or indirectly, an enzyme, polypeptide, or cofactor in the plasmin-mediated fibrinolysis cascade, such as by activating certain enzymes (e.g., lytic enzymes or activators of lytic enzymes) or by inhibiting the inhibitors of certain enzymes (e.g., inhibiting lytic enzyme inhibitors). Thrombolytics for use in the present invention can include tissue plasminogen activator (natural or recombinant), urokinase, plasminogen activators (e.g., vampire bat plasminogen activator), streptokinase, staphylokinase, prourokinase, anisolated streptokinase plasminogen activator complex (ASPAC), and animal salivary gland plasminogen activator.

In another embodiment, a fibrin-targeted peptide can be linked to a fibrinolytic. Fibrinolytics act more directly to lyse fibrin multimeric structures in clots and include, but are not limited to, copperhead snake fibrolase. Such enzymes and proteins are available commercially, or may be obtained from natural sources or tissues or by recombinant protein technology.

Anticoagulants can also be employed. Anticoagulants can affect, directly or indirectly, enzymes, cofactors, or polypeptides involved in the thrombin coagulation cascade. For example, an anticoagulant can inhibit enzymes or polypeptides that activate thrombin, or can inhibit thrombin directly. Examples include thrombin inhibitor (e.g., melagatran), Factor Xa inhibitor, tissue factor inhibitor, Factor VIIa inhibitor, Factor IXa inhibitor, Factor Va inhibitor, Factor XIa inhibitor, Factor XIIa inhibitor, TAFIα inhibitor, α2-antiplasmin inhibitor, PAI-1 inhibitor, PAI-2 inhibitor, PAI-3 inhibitor, prothrombinase inhibitor, tick anticoagulation peptide, protein C, warfarin, heparin, lepirudin, aspirin, ticlopidine, clopidogrel, tirofiban, and eptifibatide.

Examples of thrombin inhibitors are: 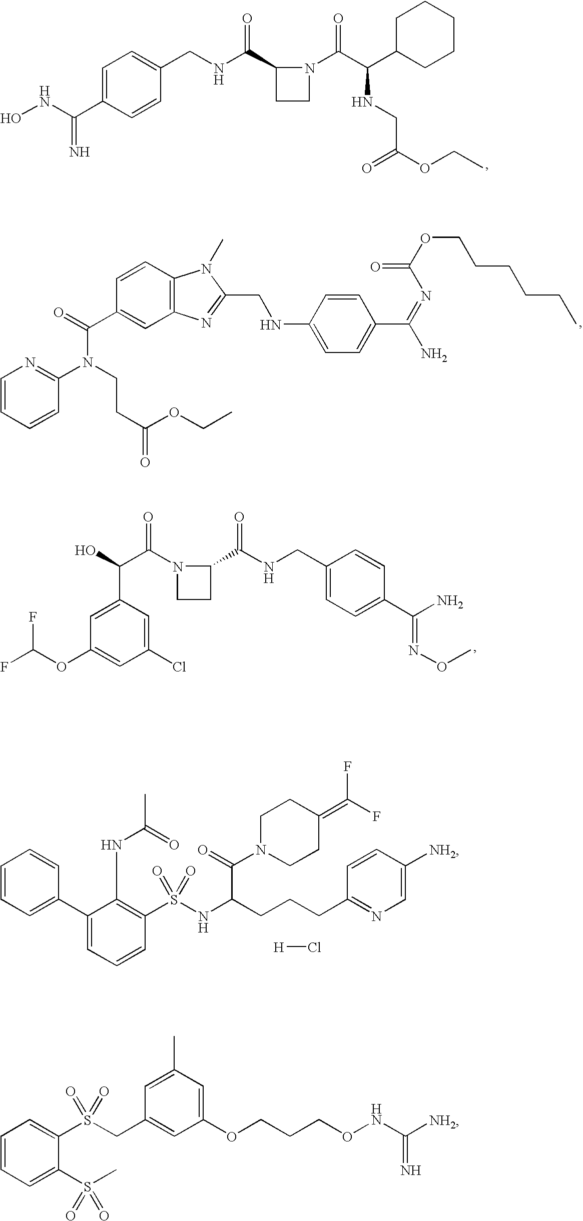 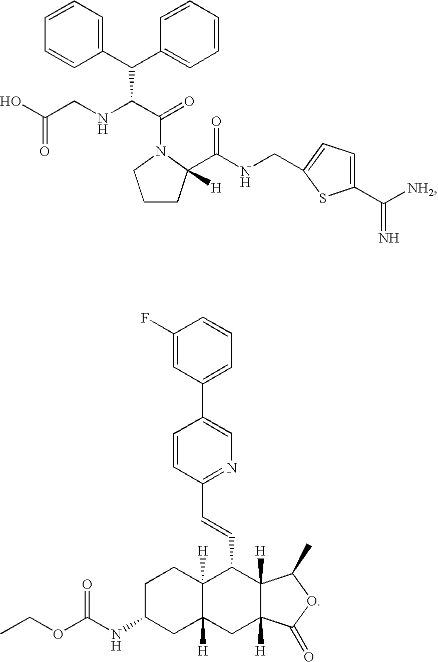
Additional thrombin inhibitors are: E-5555, MCC-977, and bivalirudin (Angiomax™).

Examples of Factor Xa inhibitors are:

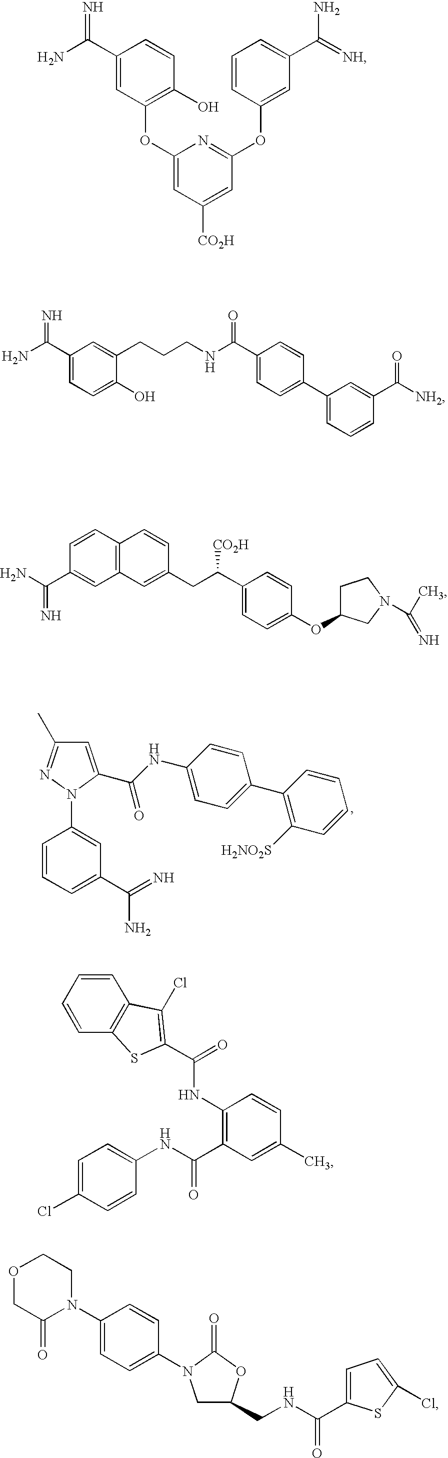

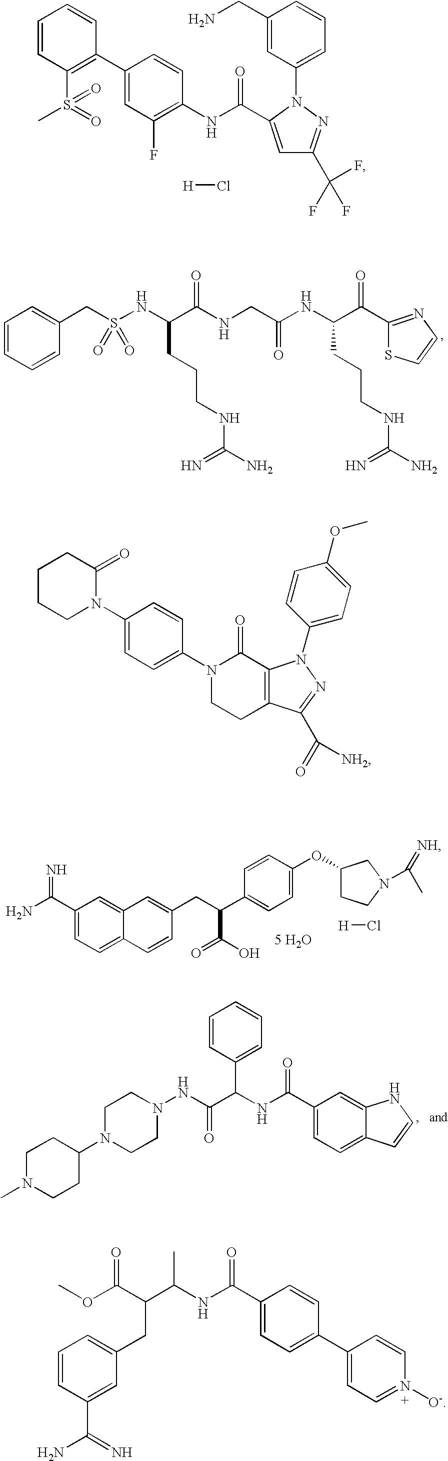

Examples of Factor Xa inhibitors are: YM-150, KFA-1982, TC-10, and 813893.

Examples of TAFIα inhibitors are:

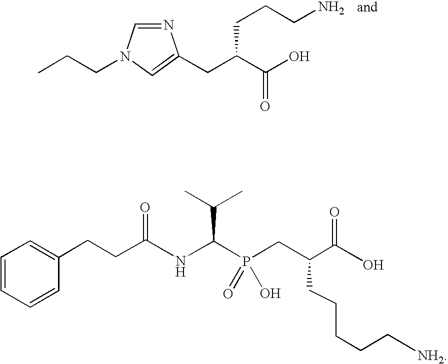

Examples of Factor VIIa inhibitors and active-site inactivated Factor VIIa are:

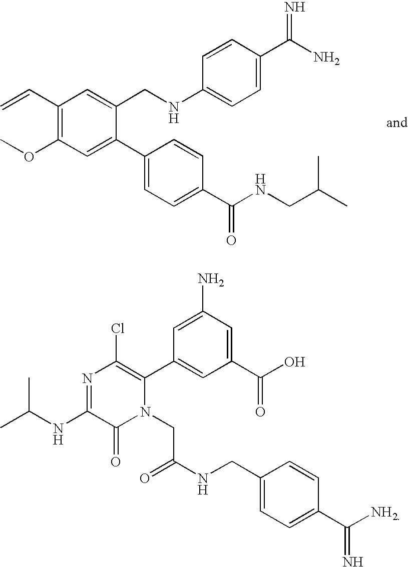

Antiplatelet moieties may inhibit platelet function, adhesion, activation, or aggregation. In addition, antiplatelet agents may inhibit secretion of prothrombotic or platelet activation factors and inhibit the recruitment and activation of monocytes. Examples include a GPIIb/IIIa receptor inhibitor, ADP receptor (e.g., P2Y₁ and P2Y₁₂) inhibitor, thrombin receptor (e.g., PAR-1 and PAR-4) inhibitor, CD40 inhibitor, CD40 L (CD40 ligand) inhibitor, Gas6 inhibitor, Gas6 receptor axl inhibitor, Gas 6 receptor inhibitor Sky, Gas6 receptor Mer inhibitor, P-selectin inhibitor, P-selectin receptor PSGL-1 inhibitor, thromboxane inhibitor, synthetase inhibitor, fibrinogen receptor antagonist, prostacyclin mimetic, phosphodiesterase inhibitor, RANTES inhibitor, phosphoinositide-3-kinase (PI(3)K) isoform β inhibitor, phosphoinositide-3-kinase (PI(3)K) isoform γ inhibitor, eptifibatide, tirofiban, ticlopidine, and clopidogrel.

Examples of GPIIb/IIIa antagonists are:

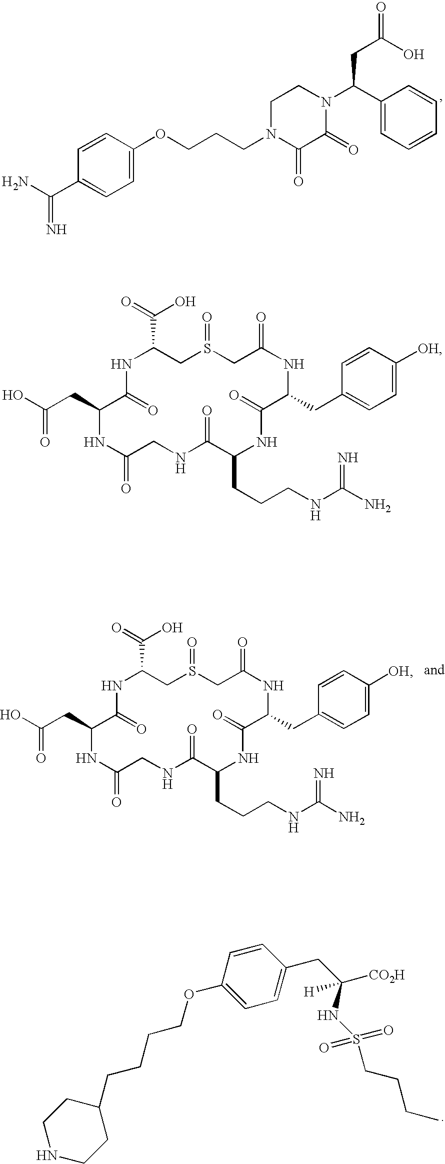

Examples of PAR-1 inhibitors include:

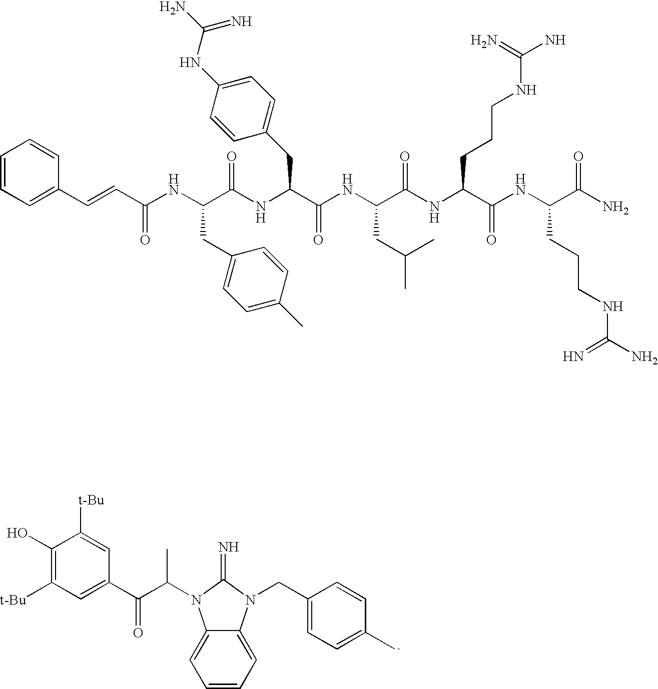

Examples of P2Y1 inhibitors include:

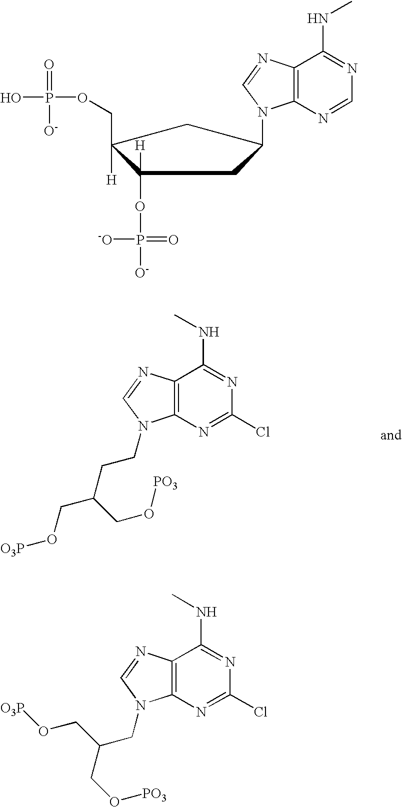

Another antiplatelet moiety inhibiting aggregation has the structure:

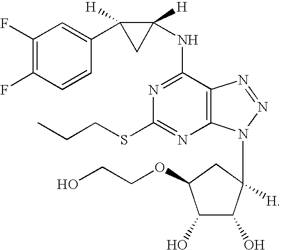

The bioactive moiety also can be any compound with general antithrombotic activity that reduces thrombus formation. Examples include hyaluronic acid and dextran.

Examples of Bioactive Conjugates

Examples of hybrid molecules include the following, where Pep is representative of a fibrin targeting peptide.
A) The Factor Xa inhibitors below can be conjugated to a fibrin-targeting moiety, such as a peptide described previously, to result in a fibrin-targeted Factor Xa inhibitor. The arrows denote possible sites of conjugation to a fibrin targeting moiety. 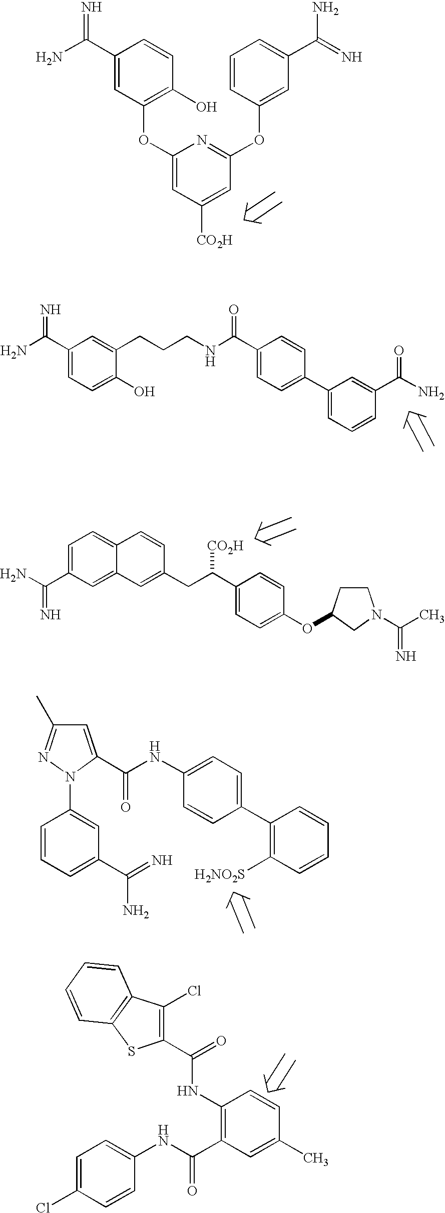
B) Where bioactive moiety targets GPIIb/IIIa: 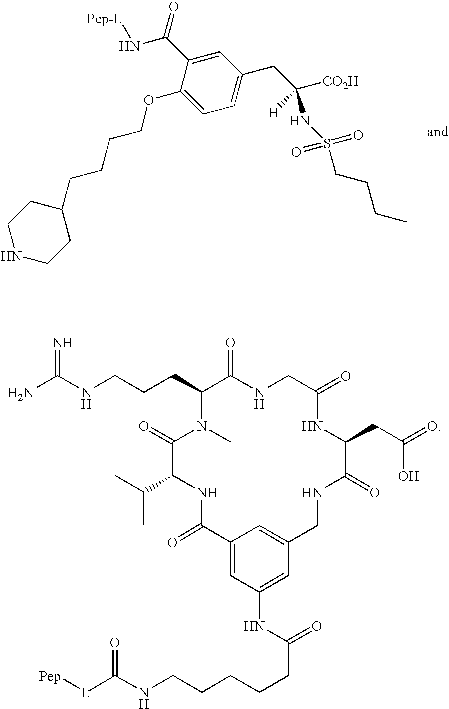
C) Where bioactive moiety targets PAR-1: 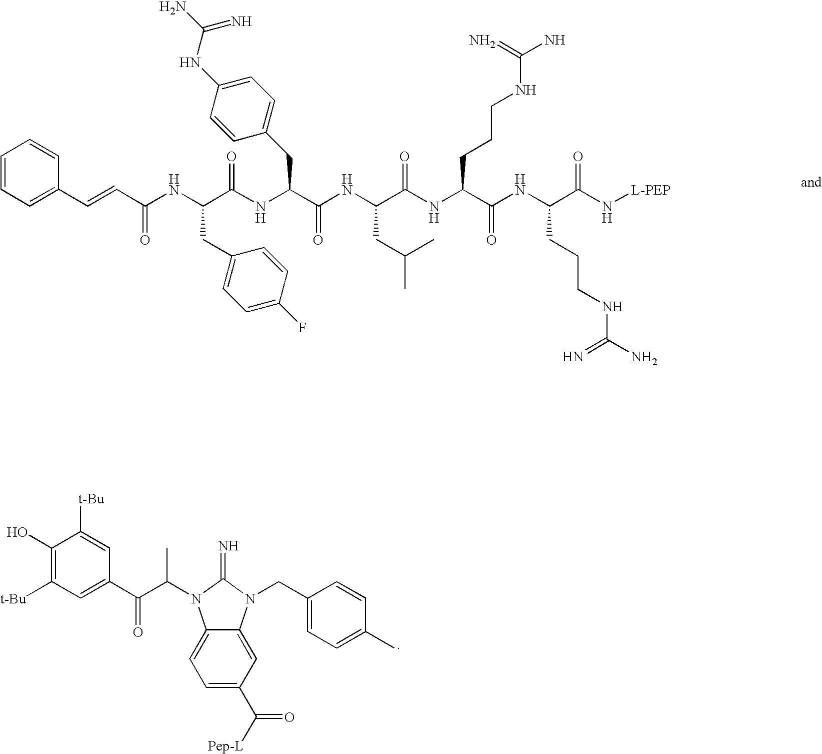
D) Where bioactive moiety is a P2Y₁ selective antagonist: 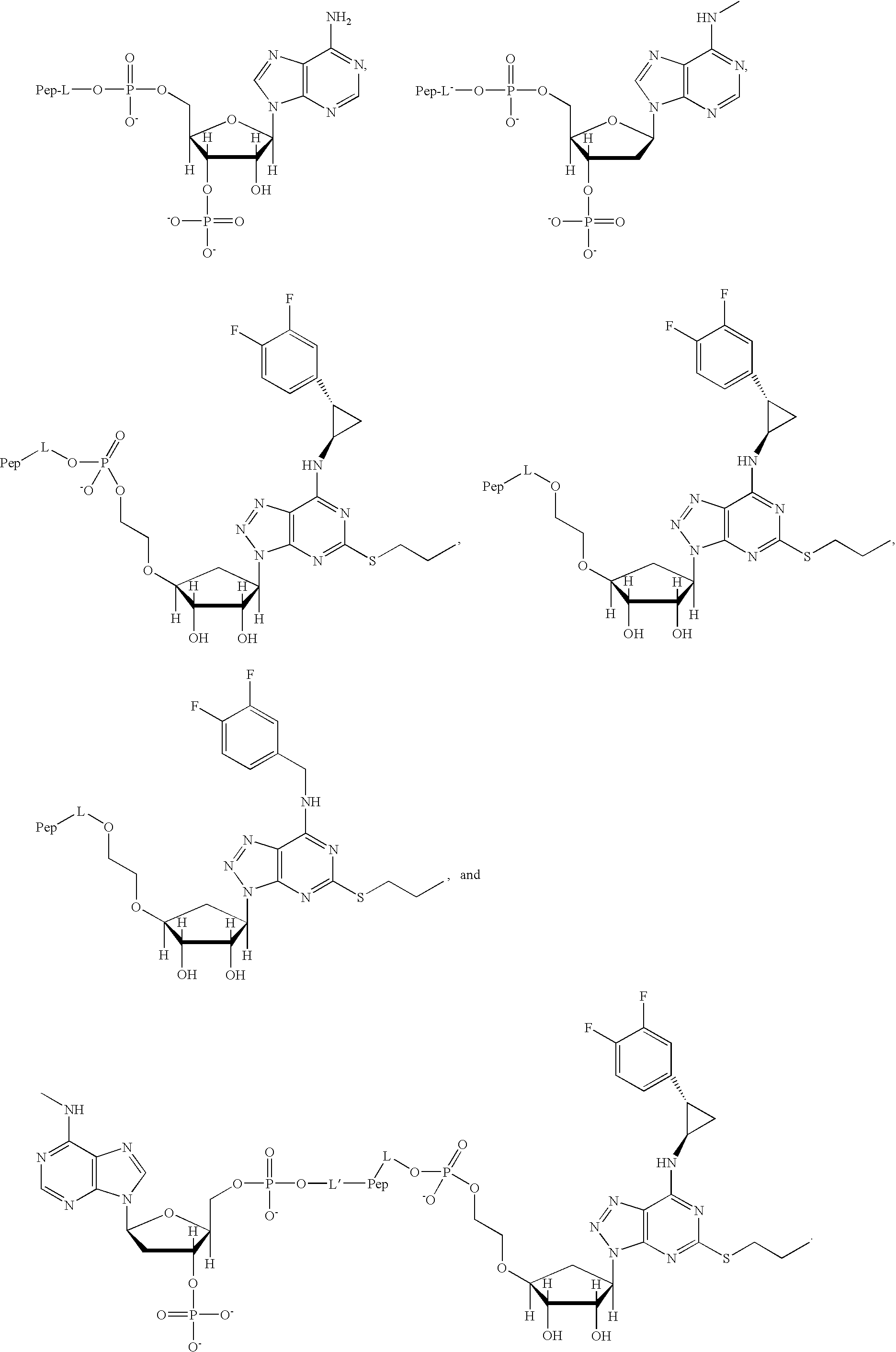
   Antibiotics

The hybrid molecules of the present invention can be effective in treating infectious diseases, such as infective endocarditis. For example, one or more fibrin-targeting moieties can be conjugated to an antibiotic. Such hybrid molecules can achieve effective antibiotic concentrations in vegetations without raising the plasma antibiotic concentrations to potentially unsafe levels. A host of currently known antibiotics can be used for conjugation with the fibrin-targeting peptide. Suitable antibiotics include a β-lactam antibiotic, sulfonamide, quinolone, trimethoprim, aminoglycoside, tetracycline, macrolide, probenecid, chloramphenicol, or glycopeptide. The β-lactam antibiotic can be a penicillin, cephalosporin, or carbapenem. The penicillin can be penicillin, ampicillin, nafcillin, oxacillin, or methicillin. The cephalosporin can be cepahlothin, cefamandole, ceftaxidime, deacetylcephalothin, cephaloridine, cefixime, or latamoxef. The macrolide can be erythromycin, clarithromycin, or azithromycin. The aminoglycoside can be kanamycin or streptomycin. The glycopeptide can be vancomycin. The technology of the current invention also applies to other classes of antibiotics. Scheme 3 provides the structure of some useful antibiotics.

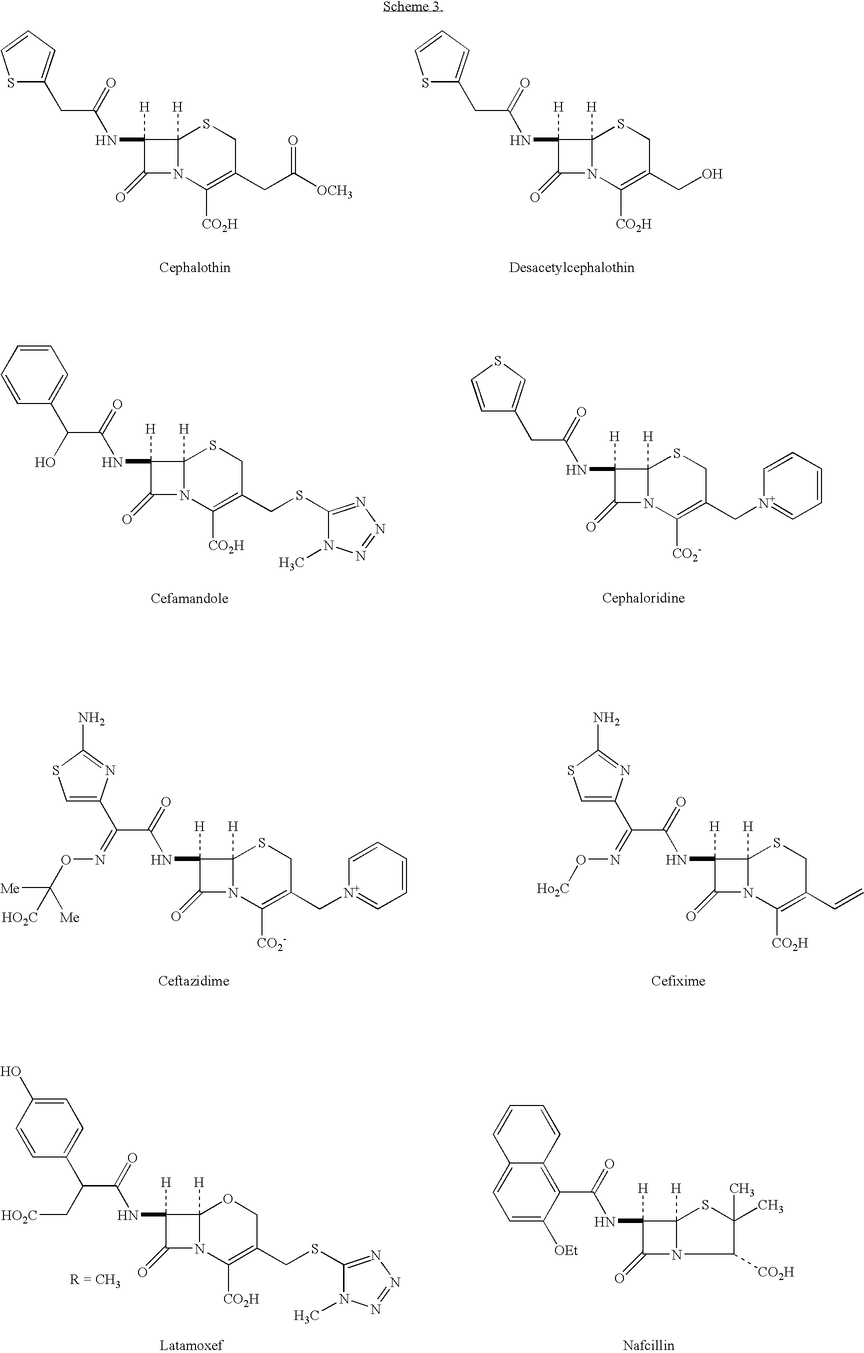

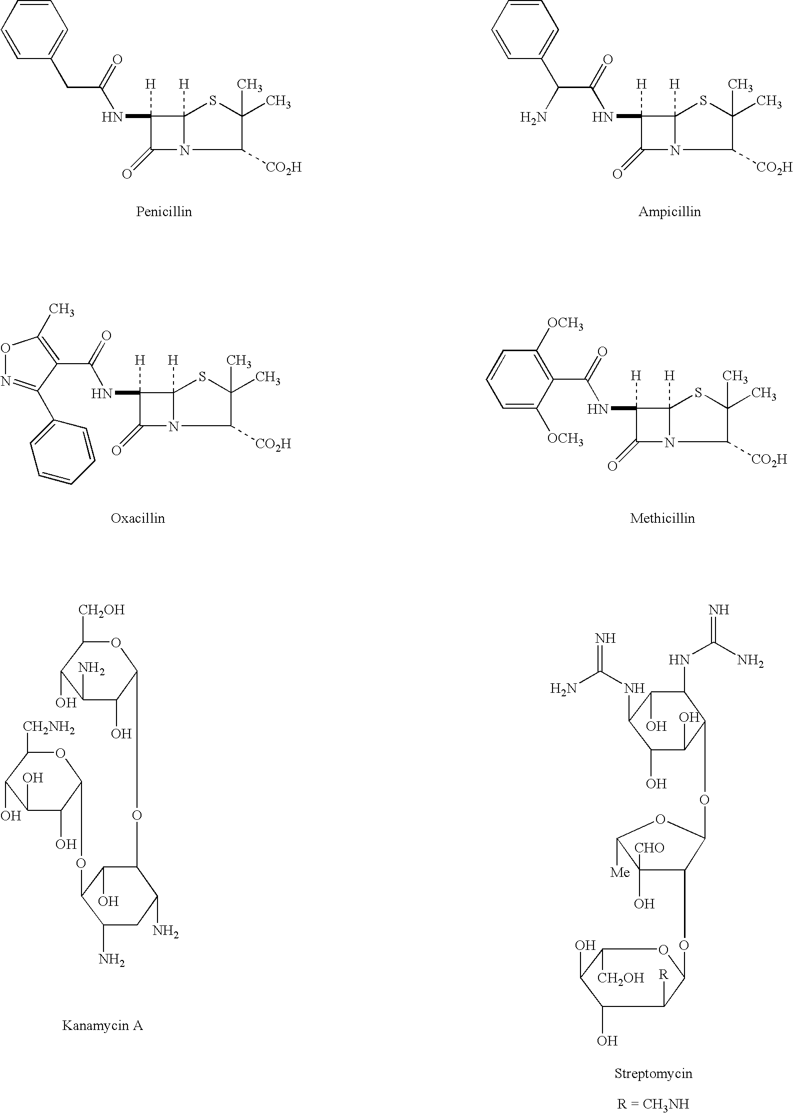

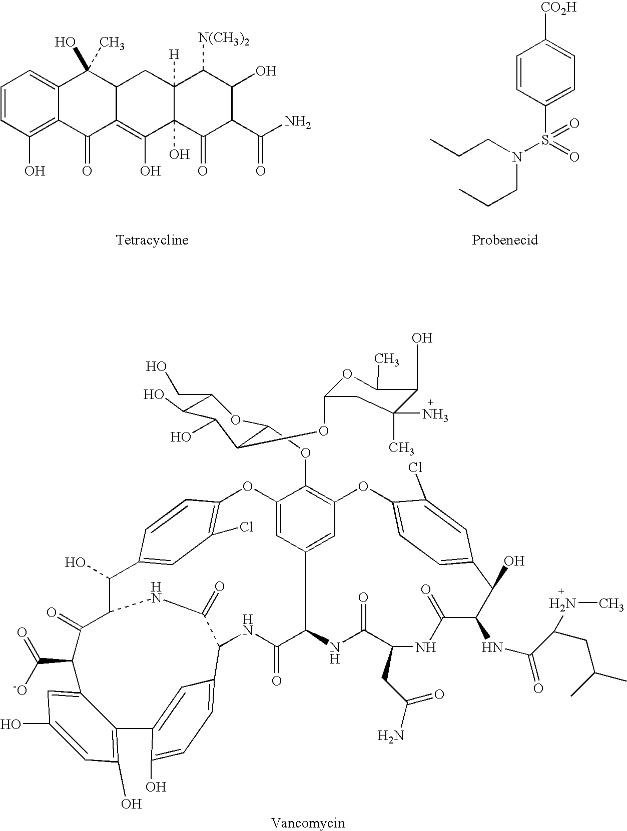

In one embodiment, fibrin-targeting moieties can be linked to β-Lactam antibiotics. β-Lactam antibiotics inhibit the synthesis of bacterial cell walls by irreversible inhibition of transpeptidases and are widely prescribed for the treatment of bacterial infections including infective endocarditis (IE) (Braunwald: Heart Disease: A Textbook of Cardiovascular Medicine, 6th ed., Copyright 2001 W. B. Saunders). β-Lactam antibiotics are peptidomimetic analogs of di- and tripeptides that mimic D-Ala-D-Ala in the transpeptidase reaction. Oral bioavailability of most of these antibiotics is quite low, and prodrug esters were developed in the past to increase oral bioavailability (Jones, K. H., et al., J. Chemotherapy 1978, 24, 217-226; Toth, I., et al., Int. J. Pharm. 1991, 73, 259-266). In some instances, chemical and metabolic stability of the esters prevented release of the free antibiotics. Double ester prodrugs were developed to circumvent this problem (Bundgaard, H. Drugs Fut. 1991, 16, 443-456; Efthymiopoulos, C., et al.,Antimicrob. Agents Chemother. 1992, 36, 1958-1963). As illustrated in Scheme 4, a hydroxymethyl ester is used in these prodrugs as the linking group to a second ester function. This general approach resulted in several commercially available prodrugs, including pivampicillin, bacampicillin, and talampicillin.

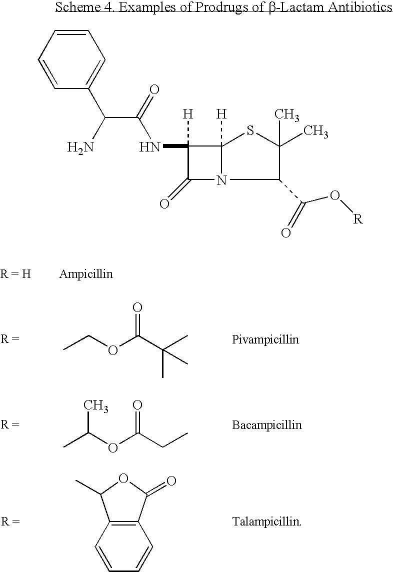

Fibrin-targeting moieties can be linked readily to compounds in the β-Lactam class of antibiotics using this double ester prodrug approach. As illustrated in Scheme 5, a fibrin binding peptide can be covalently attached to β-Lactam antibiotics. Once enzymatic cleavage of the distal ester function occurs, the residual hydroxymethyl ester rapidly decomposes to yield the parent drug. The rate of hydrolysis can be modulated by varying the distal ester moiety or the linking group (e.g., by substituting acetaldehyde with formaldehyde). A specific example of fibrin targeted antibiotics with such a double ester cleavable linker is presented in Scheme 6. Once administered, the hybrid agent can concentrate in and around the vegetation. Enzymatic cleavage of the double ester linkage releases the parent antibiotic, thereby achieving high local antibiotic concentration while maintaining a low systemic antibiotic concentration. Higher local concentration facilitates penetration of the antibiotic into the vegetation, thereby achieving better therapeutic efficacy relative to non-targeted antibiotics currently prescribed for the treatment of infective endocarditis and other types of bacterial infections. The present method also can be applied to other antibiotics having a carboxylic acid moiety for attachment of the fibrin-targeting moiety.

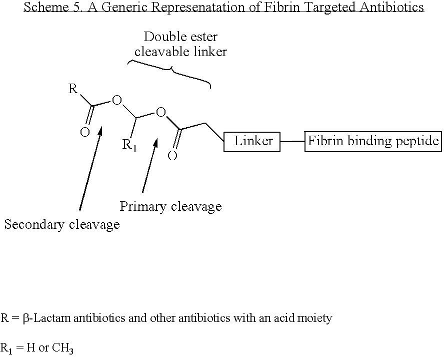

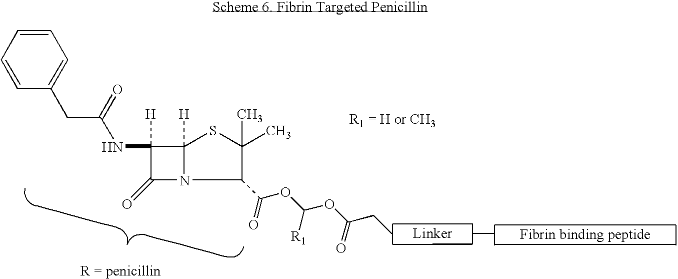

The fibrin-targeting moiety can be covalently linked to other antibiotics such as, for example, vancomycin. The glycopeptide antibiotic vancomycin is active against Gram-positive bacteria and is the drug of choice for the treatment of serious infections due to many methicillin-resistant strains including Staphylococcus aureus strains and multiply resistant strains of Streptococcus pneumoniae (Tomasz, A. N., Eng. J. Med. 1994, 330, 1247), both of which are among the leading causes of IE in adults. Vancomycin is prescribed for the treatment of IE in a variety of IE cases caused by Gram-positive bacterium strains. A variety of vancomycin analogs have been developed in order to counteract bacterial resistance. For example, vancomycin has been dimerized through a linker and exhibits enhanced potency (Sundram, U. N., et al. J. Am. Chem. Soc. 1996, 118, 13107-13108). Vancomycin has also been tethered to a polymer chain through the amino group on the sugar ring. This polymeric vancomycin has been demonstrated to be 60 times more potent than vancomycin itself (Arimoto, H., et al. Chem. Commun. 1999, 1361). Anchoring vancomycin at the site of lesion can also protect it from plasma proteases, therefore prolonging its half life and achieving a longer duration of drug action. Prolonged action vancomycin is particularly suited to IE therapy since it is known that bacteria in vegetations are particularly difficult to eradicate.

The fibrin binding moiety can be attached through a linker to the C-terminus of vancomycin. Shi et al. have demonstrated that modifications at the C-terminus of vancomycin do not alter its efficacy (Shi, Z., et al., J. Am. Chem. Soc. 1993, 115, 6482-6486). A generic representation of the prototype conjugate is illustrated in Scheme 7. 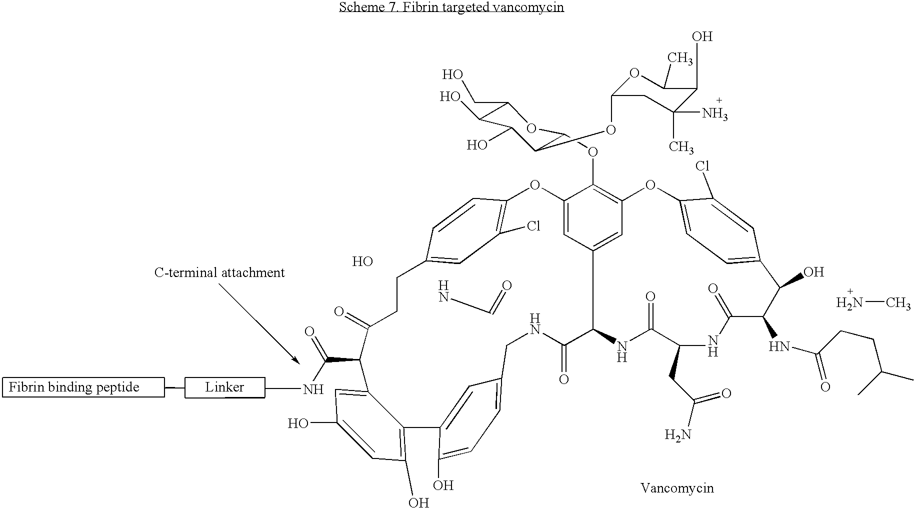
Chemotherapeutic and Antitumor Agents

The fibrin-targeting moiety can be conjugated to a chemotherapeutic or antitumor agent, such as a cytotoxin, for use in cancer therapy. Suitable cytotoxins include an alkylating agent (nitrogen mustards, nitrosoureas, or triazenes), antimetabolite (e.g., folic acid analogs, pyrimidine analogs, or purine analogs), natural product (e.g., alkaloid, ellipticine, hydroxymethylacylfulvene, epipodophyllotoxin, enzyme, biological response modifier, or hormone), antibiotic (e.g., actinomycin D, daunorubicin, doxorubicin, or idarubicin), platinum coordination complex, or antagonist (e.g., antiestrogens or antiandrogens). Exemplary cytotoxic agents are shown in Scheme 8. The present invention has the potential to reduce the toxicity of current known cancer therapeutics and improve their therapeutic indices by largely limiting the drug action to the site of the cancer. The invention also has the potential of rescuing drugs that fail clinical tests by reducing unacceptable toxicity levels of some currently disfavored cytotoxic agents to within acceptable limits.

In some embodiments, a fibrin-targeted cytotoxic agent can also act to reduce migration and proliferation of vascular smooth muscle cells and/or endothelial cells. In such cases, the fibrin-targeted cytotoxic agent can be used to prevent or inhibit stenosis or re-stenosis, e.g., after percutaneous coronary angioplasty, stent placement, or other percutaneous interventions.

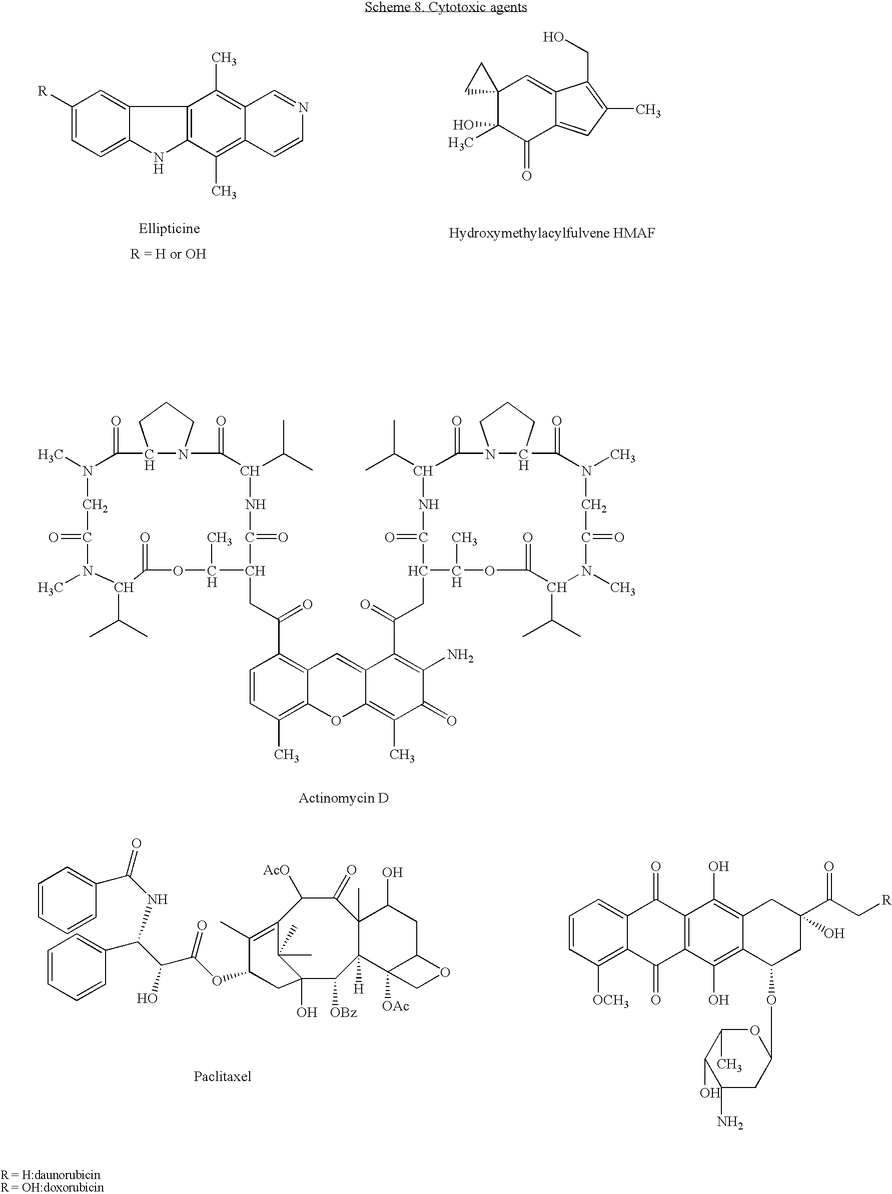

The cytotoxic agents exemplified in Scheme 8 can be covalently linked to a fibrin-targeting moiety through either a cleavable or non-cleavable linker. The cleavable linker typically includes an oligopeptide and can be about 2 to about 10 amino acid residues in length. The sequence of the linker can be such that the scissile amide bond is processed by a proteolytic enzyme, typically one that is upregulated at tumors. Such enzymes include, but are not limited to, cathepsin D, matrix metalloproteases (MMPs, especially MMP- 1, -2, -3 and -13), urokinase, and prostate specific antigen (PSA). Proteolytic processing of the cleavable linker releases the active drug at the tumor site. Exemplary hybrid molecules including cytotoxic agents are shown in Scheme 9. 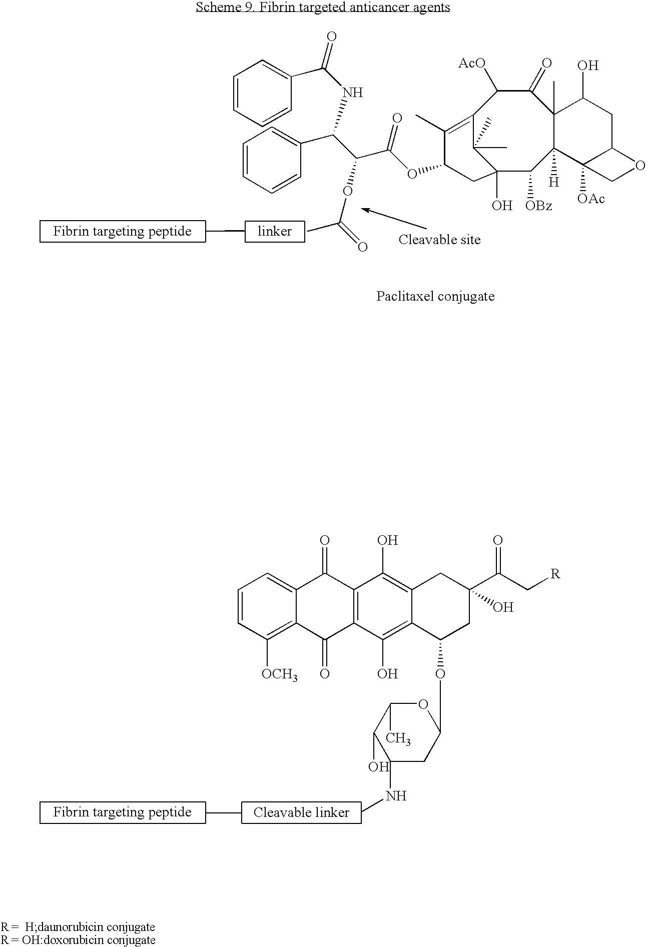
Radiopharmaceutical

A fibrin-targeting moiety can be conjugated to radiopharmaceuticals such as radionuclides for use in cancer therapy. A fibrin-targeting moiety can be labeled through the formation of stable metal complexes of radionuclides with a chelating group covalently attached to a fibrin-targeting moiety. Such high affinity metal chelating groups include, but are not limited to, DTPA, DOTA, and their variants. ¹³¹i labeled fibrin-targeting moieties can be directly labeled using iodogen (Salacinski, P. R., et al., Anal. Biochem 1981, 117, 136-146). Exemplary radionuclides are listed in Table 1. In addition, many radiolabeled agents are currently under clinical investigation, such as, for example, radiolabeled somatostatin analogs (DeJong, M., et al., Semin. Nucl. Med. 2002, 32, 133-140) and various radiolabeled monoclonal antibodies (Goldenberg, D. M. J. Nuc. Med. 2002, 43, 693-713).

TABLE 1
Therapeutic Radionuclides

| Isotope | Symbol | t_{1/2} (h) | Emission |
|---|---|---|---|
| Iodine-131 | ¹³¹I | 193 | β |
| Yttrium-90 | ⁹⁰Y | 64 | β |
| Lutetium-177 | ¹⁷⁷Lu | 161 | β |
| Copper-67 | ⁶⁷Cu | 62 | β |
| Rhenium-186 | ¹⁸⁶Re | 91 | β |
| Rhenium-188 | ¹⁸⁸Re | 17 | β |
| Bismuth-212 | ²¹²Bi | 1 | β |
| Bismuth-213 | ²¹³Bi | 0.77 | β |
| Astatine-211 | ²¹¹At | 7.2 | β |

Examples of radiolabeled fibrin-targeting agents are given in Scheme 10. 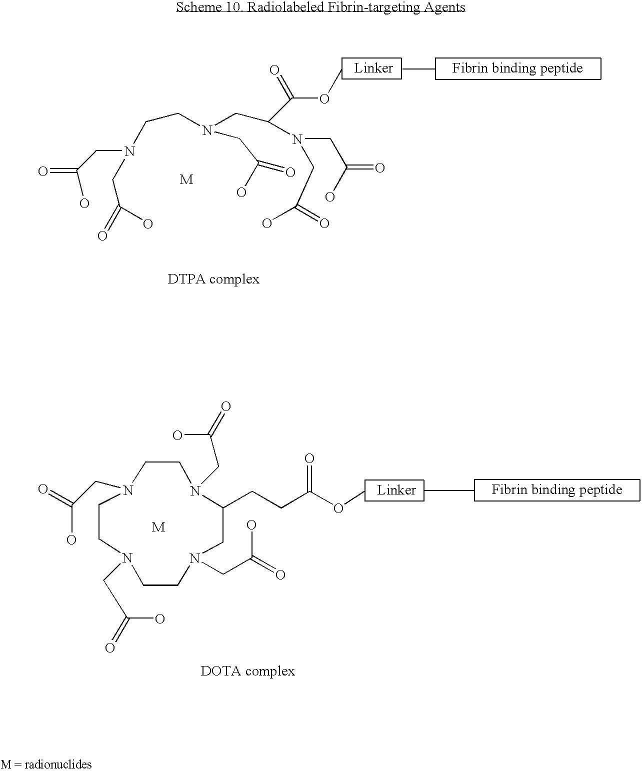
Liposomes

The therapeutic efficacy of many drugs can be improved by encapsulating the drug in liposomes (Danilo D. Lasic and David Needham Chemical Reviews 1995, 95, 2601-2628). Thus, a bioactive moiety can be a drug delivery vehicle loaded with therapeutic or diagnostic agents. Liposomes are widely used for drug delivery. Liposomes conjugated to a fibrin binding moiety can be used to actively target thromboembolism, infection, and cancer. Fibrin-targeting liposomes are also useful in the delivery of antithrombotics, antibiotics, antitumor or chemotherapeutic agents, or radiopharmaceuticals. The agents can be entrapped in the inner space, bound to the inner membrane, bound to the outer membrane, partially inserted in the membrane bilayer, spanning the bilayer, or dissolved in the bilayer. A generic representation of these liposome constructs is given in Scheme 11. 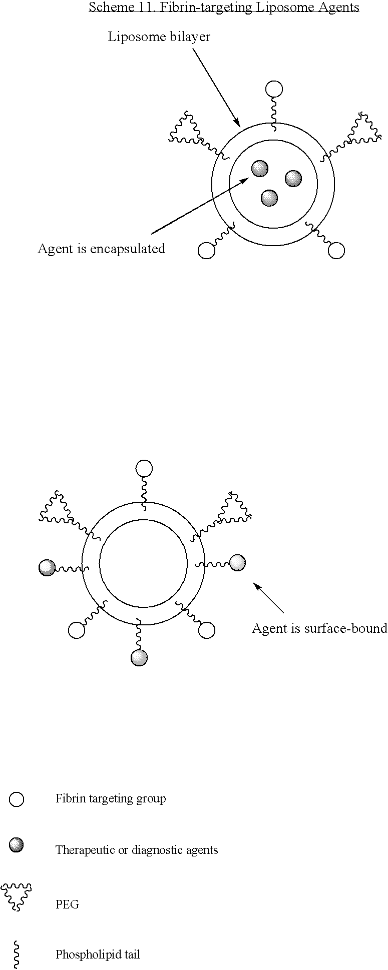
Modifying Moiety

A “modifying moiety” refers to a moiety used to adjust the physical, physiological and/or biological properties of the resulting hybrid molecule, including solubility, stability, or detectability. For example, a “modifying moiety” may be an imaging agent to provide visualization, e.g., a radiolabeled molecule, a microsphere, or a paramagnetic metal containing chelate.

In some embodiments, a modifying moiety can be used to adjust the solubility of the hybrid molecule. Examples include chelating ligands (e.g., DTPA, DOTA), which may be optionally conjugated to paramagnetic metal ions such as Gd(III), PEG moieties, polysaccharides, acidic moieties, salts, and esters.

Therapeutic Applications

It is well established that beneficial therapeutic effects of pharmaceuticals are derived from the active drug at the site of drug action (Pharmacology-Drug Actions and Reactions, Ruth R. Levine, Parthenon Publishing, 6^{th} Edition). It can be necessary to achieve and maintain a certain plasma drug concentration for a non-targeted drug to reach the site of action by passive diffusion and reside there for a long enough period of time to be therapeutically beneficial. The hybrid molecules described herein provide compounds that may be localized and retained at the site of action without relying solely on passive diffusion to deliver the therapeutic agent to the site of action. These hybrid therapeutic agents can effectively deliver drugs to the diseased tissues, thus raising the local drug concentration at the desired site of drug action. Furthermore, the hybrid molecules can be retained in the diseased tissues for a longer period of time than non-targeted therapeutics due to their binding to the polymeric fibrin meshwork within or around the diseased tissues, thus allowing for longer durations of drug action. This “concentrating” or “homing” effect may reduce the need to maintain higher plasma levels of the therapeutic due to metabolism of the active drug moiety from circulating proteolytic enzymes, and in tissues through which the blood supply circulates, e.g. liver, thereby improving safety profiles relative to non-targeted therapeutics.

The present invention can “rescue” drugs that fail clinical tests by reducing unacceptable systemic toxicity, such as systemic bleeding of some currently disfavored non-targeted or systemic therapeutic agents, to within acceptable limits.

In addition to its effects on the coagulation process, thrombin is known to activate a large number of cells such as neutrophils, fibroblasts, endothelial cells and smooth muscle cells. Therefore, the hybrid molecules of the invention may be also useful for the therapeutic and/or prophylactic treatment of idiopathic and adult respiratory distress syndrome, pulmonary fibrosis following treatment with radiation or chemotherapy, pulmonary fibrosis, renal fibrosis, hepatic fibrosis, septic shock, lupus erythematosus, septicemia, acquired thrombophilic disorders, congenital thrombophilic disorders, venous thromboembolism, thrombosis secondary to other disease states or syndromes, orthopedic surgery, elective surgery, general surgery where there is risk of thrombosis requiring prophylactic treatment with antithrombotic agents, inflammatory responses, which include, but are not limited to acute or chronic atherosclerosis such as coronary arterial disease, cerebral arterial disease, peripheral arterial disease, reperfusion damage, and restenosis after percutaneous trans-luminal angioplasty.

Hybrid molecules of the invention can be used for the therapeutic and/or prophylactic treatment of antiphospholipid antibodies, homocysteinemia, heparin induced thrombocytopenia, venous thrombosis, deep venous thrombosis, pulmonary embolism, arterial thrombosis, myocardial infarction, unstable angina, thrombosis-based cerebral stroke, peripheral arterial embolism, systemic embolism, septic shock, pancreatitis, elective hip arthroplasty, elective knee arthroplasty, coronary artery bypass graft surgery, restenosis after percutaneous trans-luminal angioplasty for treatment of peripheral artery disease, restenosis after percutaneous trans-luminal angioplasty for treatment of coronary artery disease, limb ischemia, stroke prevention in patients with atrial fibrillation, myocardial infarction with ST elevation, non-ST elevation myocardial infarction (unstable angina), Factor V Leiden, Protein C deficiency, Protein S deficiency, elevated levels of prothrombotic blood coagulation factors (e.g. Factor VIII), elevated levels of antithrombolytic factors (e.g. plasminogen activator inhibitor type 1), depressed levels of coagulation inhibitory or thrombolytic factors (e.g. antithrombin), hormonal therapy (e.g. hormone replacement therapy), age related thrombosis, immobility, thrombosis secondary to malignancy or cancer therapy, and immune form of heparin-induced thrombocytopenia.

The hybrid molecules of the invention may be also combined and/or co-administered with an anticoagulant agent such as a thrombin inhibitor (e.g., melagatran), Factor Xa inhibitor, tissue factor inhibitor, Factor VIIa inhibitor, Factor IXa inhibitor, Factor Va inhibitor, Factor XIa inhibitor, Factor XIIa inhibitor, TAFIα inhibitor, α2-antiplasmin inhibitor, PAI-1 inhibitor, PAI-2 inhibitor, PAI-3 inhibitor, prothrombinase inhibitor, tick anticoagulation peptide, protein C, warfarin, heparin, lepirudin, aspirin, ticlopidine, clopidogrel, tirofiban, and eptifibatide.

The hybrid molecules of the invention may be also combined and/or co-administered with antiplatelet agents such as a GPIIb/IIIa receptor inhibitor, ADP receptor (e.g., P2Y₁ and P2Y₁₂) inhibitor, thrombin receptor (e.g., PAR-1 and PAR-4) inhibitor, CD40 inhibitor, CD40L (CD40 ligand) inhibitor, Gas6 inhibitor, Gas6 receptor axl inhibitor, Gas 6 receptor inhibitor Sky, Gas6 receptor Mer inhibitor, P-selectin inhibitor, P-selectin receptor PSGL-1 inhibitor, thromboxane inhibitor, synthetase inhibitor, fibrinogen receptor antagonist, prostacyclin mimetic, phosphodiesterase inhibitor, RANTES inhibitor, phosphoinositide-3-kinase (PI(3)K) isoform β inhibitor, phosphoinositide-3-kinase (PI(3)K) isoform γ inhibitor, eptifibatide, tirofiban, ticlopidine, and clopidogrel.

The hybrid molecules of the invention may also be further combined and/or co-administered with fibrinolytics such as copperhead snake fibrolase.

The hybrid molecules of the invention may further be combined and/or co-administered with thrombolytics such as tissue plasminogen activator (natural or recombinant), urokinase, plasminogen activators (e.g., vampire bat plasminogen activator), streptokinase, staphylokinase, prourokinase, anisolated streptokinase plasminogen activator complex (ASPAC), and animal salivary gland plasminogen activator, and the like, for the treatment of thromboembolism, in particular myocardial infarction.

Pharmaceutical Compositions

The therapeutic agents of the present invention can be formulated in accordance with routine procedure known in the art as pharmaceutical compositions adapted for human or animal (e.g., mammal) patients. As used herein, a mammal can be a human, monkey, mouse, rat, horse, pig, dog, cat, or rabbit. Where necessary, the formulation can include pharmaceutically acceptable ingredients, such as solubilizing agents, excipients, carriers, adjuvants, vehicles, preservatives, a local anesthetic, flavorings, colorings, and the like. The ingredients may be supplied separately, e.g., in a kit, or mixed together in a unit dosage form. The dosage to be administered and the mode of administration will depend on a variety of factors including sex, age, weight, condition of the patient, metabolic profile of the patient, genetic factors, and the like.

The pharmaceutical compositions of the current invention may be administered by a variety of conventional ways, including both oral and parenteral administration. Parenteral administration includes, but is not limited to, subcutaneous, intravenous, intraarterial, interstitial, intrathecal, and intracavity administration. Intravenous and subcutaneous administration is particularly useful.

For intravenous administration, pharmaceutical compositions may be given as a bolus, as two or more doses separated in time, or as a constant or non-linear flow infusion. Pharmaceutical dosage forms suitable for intravenous or subcutaneous administration of such pharmaceutical compositions can involve a sterile solution or a lyophilized powder. In the case of a lyophilized powder, the dosage forms can be reconstituted with water or saline for injection before administration to a patient. For oral administration of these pharmaceutical compositions, dried powder or blends, tablets, capsules, liquid solutions or suspensions may be used.

General Strategies for Synthesis of Hybrid Molecules

Some general strategies for synthesizing the hybrid molecules described herein are presented below. The round sphere represents a resin bead on which a peptide is synthesized. Amino acids have the abbreviations described previously, and may be protected by protecting groups, e.g., tBu, Acm, Trt, Dab. The lines between two cysteines (C's) represents a disulfide linkage. [Z] is a modification moiety, such as a chelating ligand, optionally having a paramagnetic metal bound thereto; [L] is a linker.

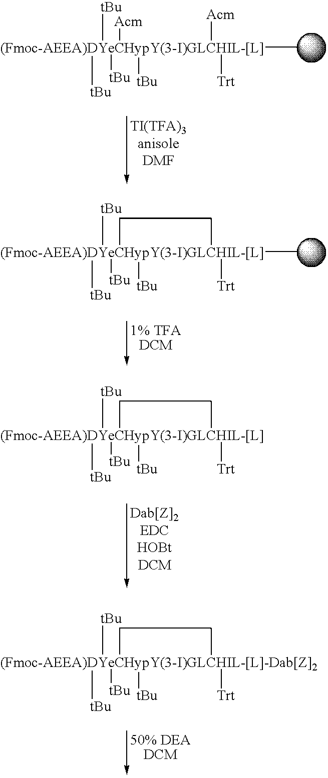

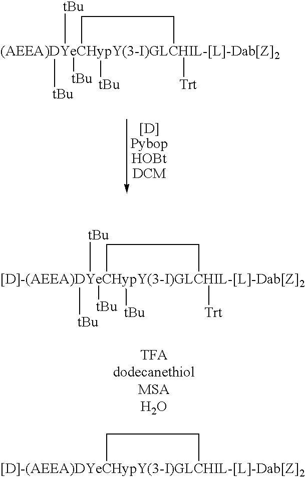

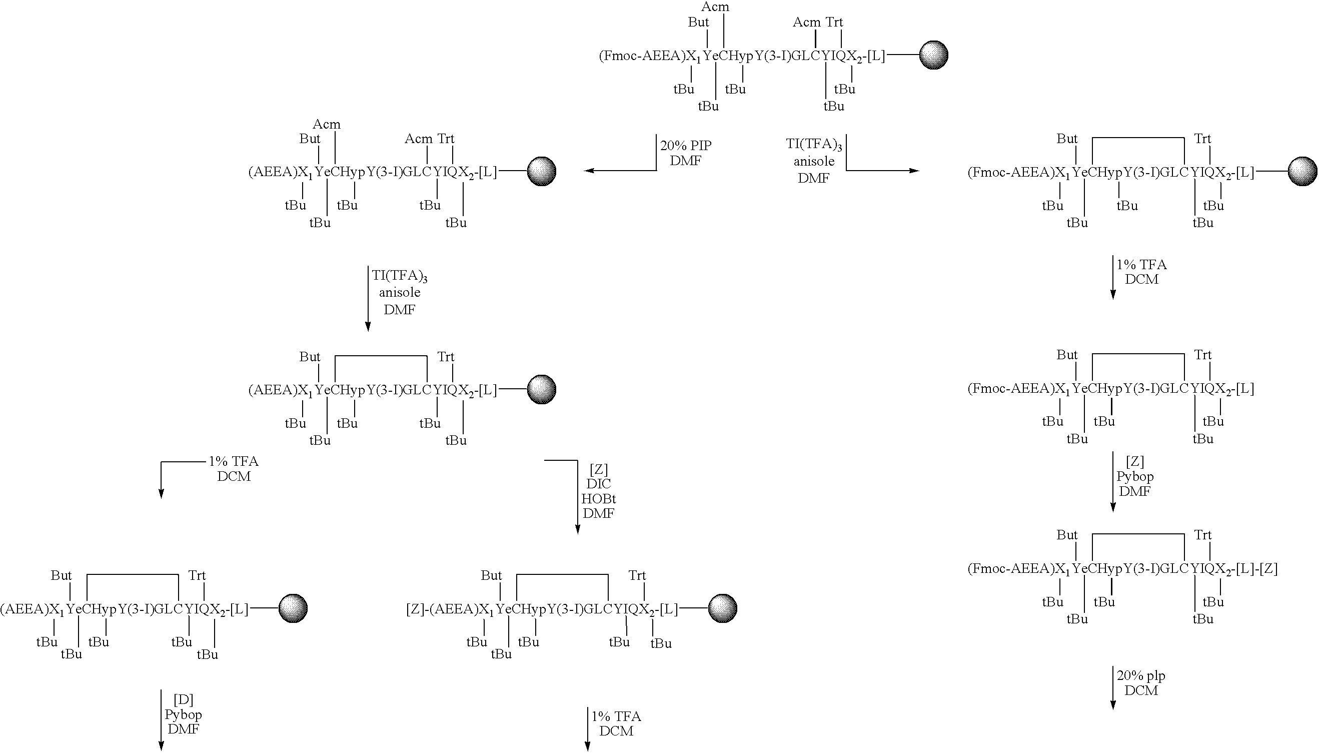

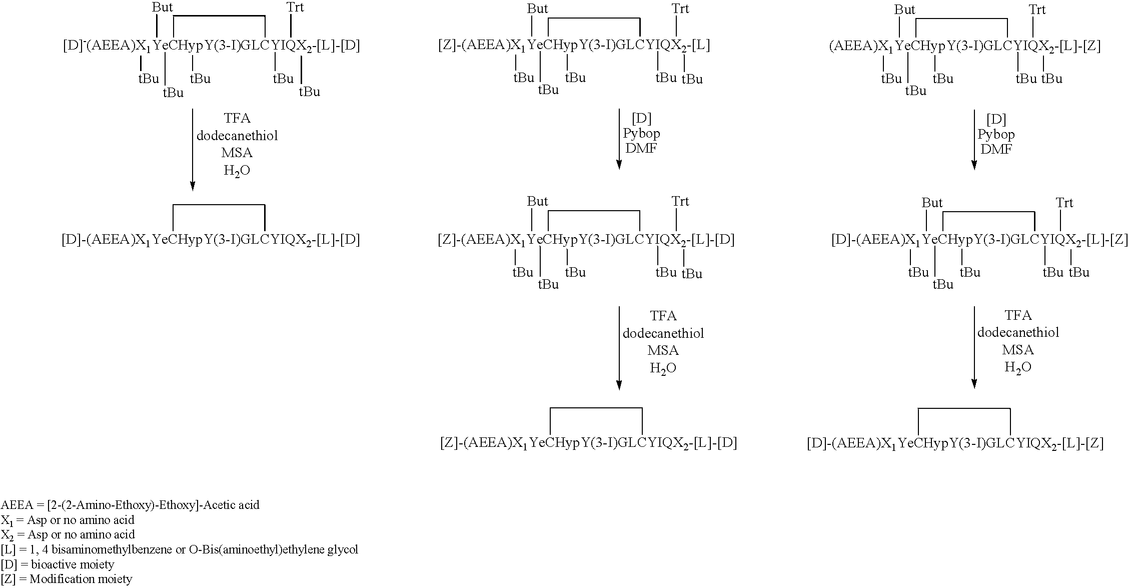

### EXAMPLES

Fibrin targeted therapeutics are further illustrated in the following examples. The specific hybrid molecules and molecular parameters such as molecular structures, molecular size, key substituents, the manner of attachment (e.g., C-terminus vs. N-terminus), and the like incorporated in these examples are intended to illustrate the practice of the invention, and they are not presented to in any way limit the scope of the invention.

### Example No. 1

### Synthesis of Fibrin Targeting Peptide

A peptide is synthesized on an automated peptide synthesizer using continueous flow. 2 mmol of commercially available 1,4 (bisaminomethyl) benzene trityl PEG resin (˜0.20 mmol/g) or O-Bis(aminoethyl)ethylene glycol trityl peg resin (˜0.2 mmol/g) is loaded onto the pioneer peptide synthesizer. A 3-fold excess of the following amino acids is used to synthesize the peptide on the resin:
1.* Fmoc-Asp(OtBu)-OH
1. Fmoc-Gln(Trt)-OH
2. Fmoc-Ile-OH
3. Fmoc-Tyr(tBu)-OH
4. Fmoc-Cys(Acm)-OH
5. Fmoc-Leu-OH
6. Fmoc-Gly-OH
7. Fmoc-Tyr(3-I)-OH
8. Fmoc-Hyp(tBu)-OH
9. Fmoc-Cys(Acm)-OH
10. Fmoc-D-Glu(OtBu)-OH
11. Fmoc-Tyr(tBu)-OH
11.* Fmoc-Asp(OtBu)-OH
12. Fmoc-8-amino-3,6-dioxaoctanoic acid

Standard Fmoc chemistry is used to elongate the peptide on the resin. The fmoc group is removed with a solution of 20% piperidine in dimethylformamide. Each amino acid is coupled to the peptide using a 0.5 M solution of diisopropylcarbodiimide in dimethylformamide and a 0.5 M solution of 1-hydroxybenzotriazole in dimethylformamide. The completed peptide/resin is washed with 2-propanol.

After the synthesis of the peptide on the resin is complete, the peptide is cyclized on the resin using the following cyclization conditions. The resin is placed in a 500 mL peptide reaction vessel and washed 2× with 300 mL of dimethylformamide. The resin is then suspended in 450 mL of 5% anisole in dimethylformamide. 1.2 equivalents of thallium trifluoroacetate are added. The resin is shaken for 18 h at room temperature. The resin is then washed with 300 mL of the following: DMF 2×, MeOH 1×, DCM 3×.

The cyclized peptide on the resin is then cleaved from the resin using the following cleavage cocktail: 1% Trifluoroacetic acid in dichloromethane. The resin is suspended in 100 mL of 1 % TFA in DCM and shaken for 2 minutes. The resin is then filtered. The filtrate is neutralized with 20 mL of 10% pyridine in methanol. This process is repeated 20 times. The neutralized filtrates are then combined and concentrated under vacuum to a volume of 250 mL. Water (750 mL) is then added to the concentrated solution and a white precipitate forms. The precipitate is collected by filtration. The crude protected peptide is then purified by prep HPLC and lyophilized to give the final peptide moiety.

### Example No. 2

Preparation of a Fibrin Localized Thrombin Inhibitor Bisconjugate (2 Bioactive Moieties per 1 Peptide)—Melagatran

A bisconjugate was prepared as shown in the general scheme below: 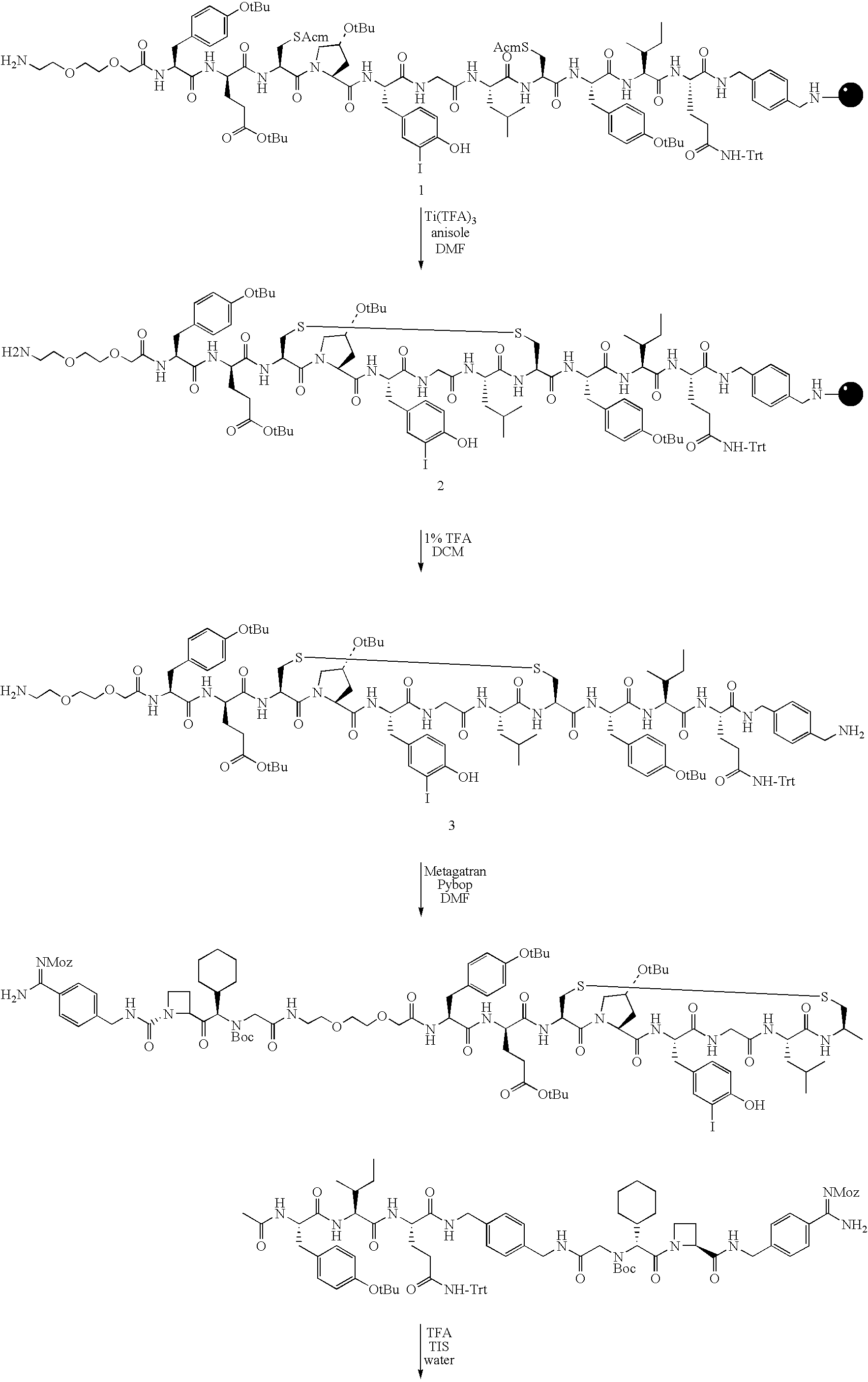 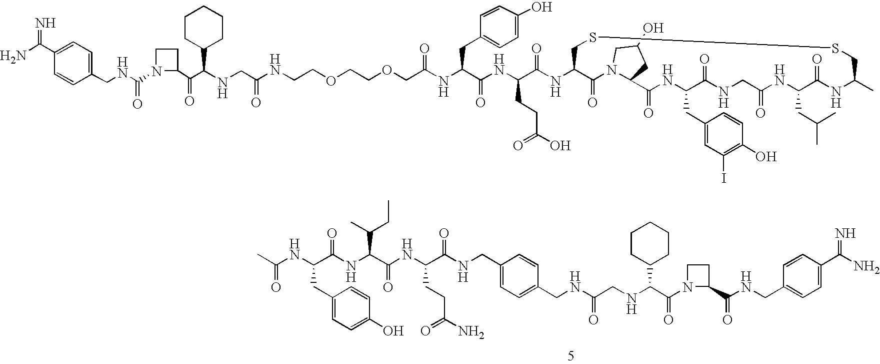 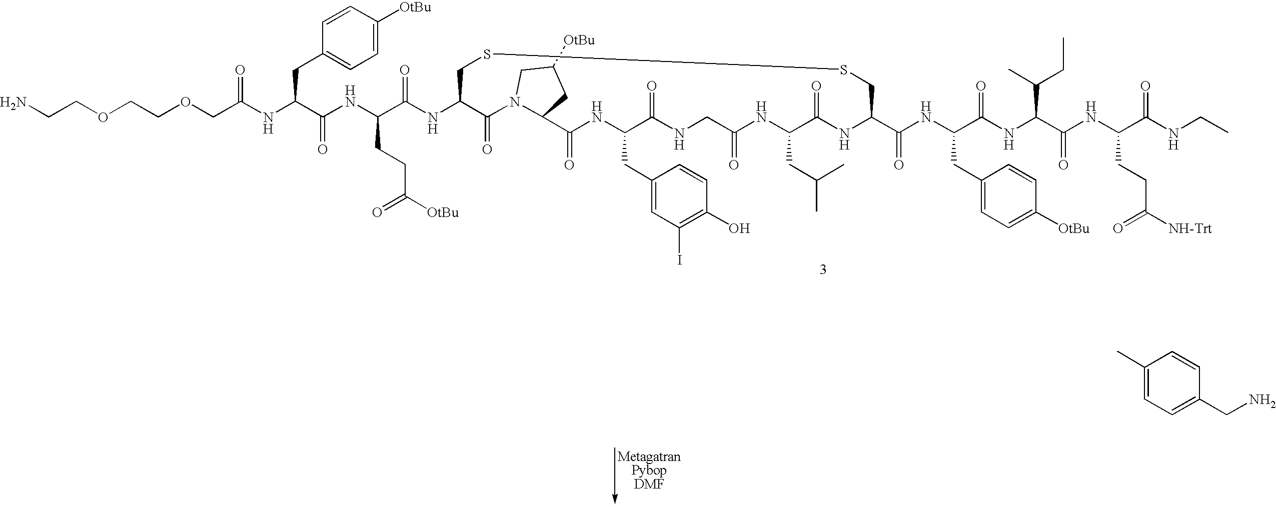
Compound 2

Suspend the peptide on resin (compound 1) in 450 mL of 5% anisole in dimethylformamide. Add thallium trifluoroacetate (1.3 g, 2.4 mmol). Shake the resin overnight for 18 hr. Wash the resin with 300 mL of DMF 2×, MeOH 1×, DCM 3×.

Compound 3

Suspend the peptide-resin (compound 2) in 100 mL of 1% TFA in DCM and shaken for 2 minutes. Filter the resin. Neutralize the filtrate with 20 mL of 10% pyridine in methanol. Repeat this process 20 times. Combine the neutralized filtrates and concentrate under vacuum to remove all dichloromethane. Add water (750 mL) to the concentrated solution to form a white precipitate. Filter the precipitate, wash with water, and drive to give crude compound 3. Purify the crude protected peptide by prep HPLC and lyophilize to give 860 mg of purified peptide 3.

m/e 1111.4 (M+2H/2)

Compound 4

Dissolve compound 3 (190 mg, 0.0855 mmol) in dimethylformamide (2 mL). Add triethylamine (47.6 μL, 0.171 mmol). Dissolve protected melagatran (118.7 mg, 0.171 mmol) and pybop (89 mg, 0.171 mmol) in dimethylformamide (3 mL) and add to the peptide solution. Stir the reaction for 18 hr at room temperature. Concentrate the reaction under vacuum to a volume of ˜1 mL. Add water (50 mL) to form a white precipitate. Filter the precipitate, wash with water, and dry to give 196 mg of compound 4. Use without any further purification in the next step.

m/e 1787.2 (M+2H/2)

Compound 5

Dissolve compound 4 (196 mg, 0.0549 mmol) in 5 mL of 90% trifluoroacetic acid, 5% triisopropylsilane, 5% water. Stir the reaction for 2 hr at room temperature. Concentrate the reaction to an oil under vacuum. Add ether to form a precipitate. Filter the precipitate, wash with ether, and dry under vacuum to give 170 mg of crude compound 5. Purify the crude product by prep HPLC and lyophilize to give 121 mg of purified compound 5. Compound 5 is also known as melagatran.

m/e 1289.6 (M+2H/2)

### Example No. 3

### Preparation of Hybrid Molecule 1 Containing a Fibrin-Localized Thrombin Inhibitor (Melagatran) with a Modification Group

Hybrid molecule 1 was prepared according to the general scheme set forth below: 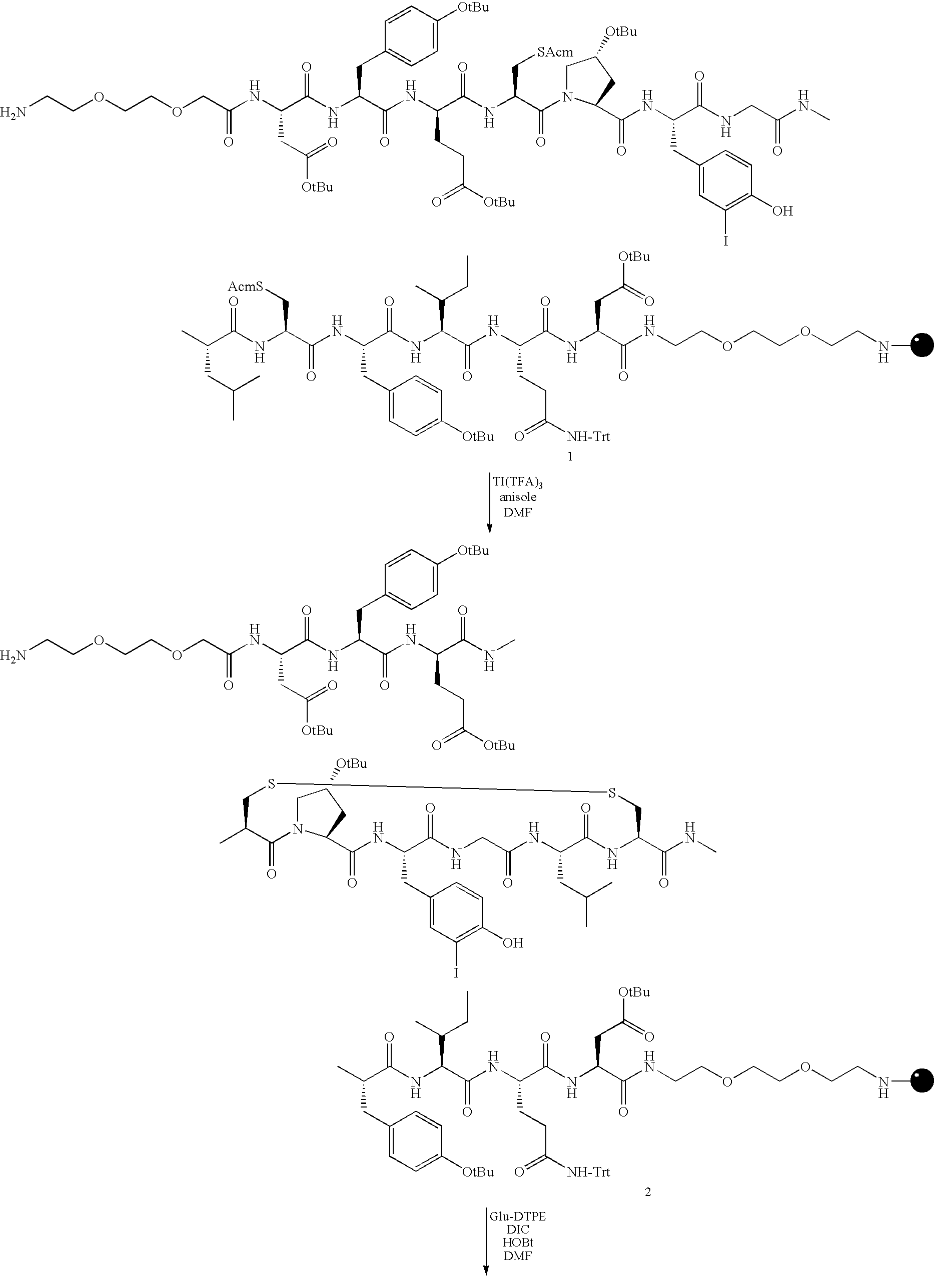 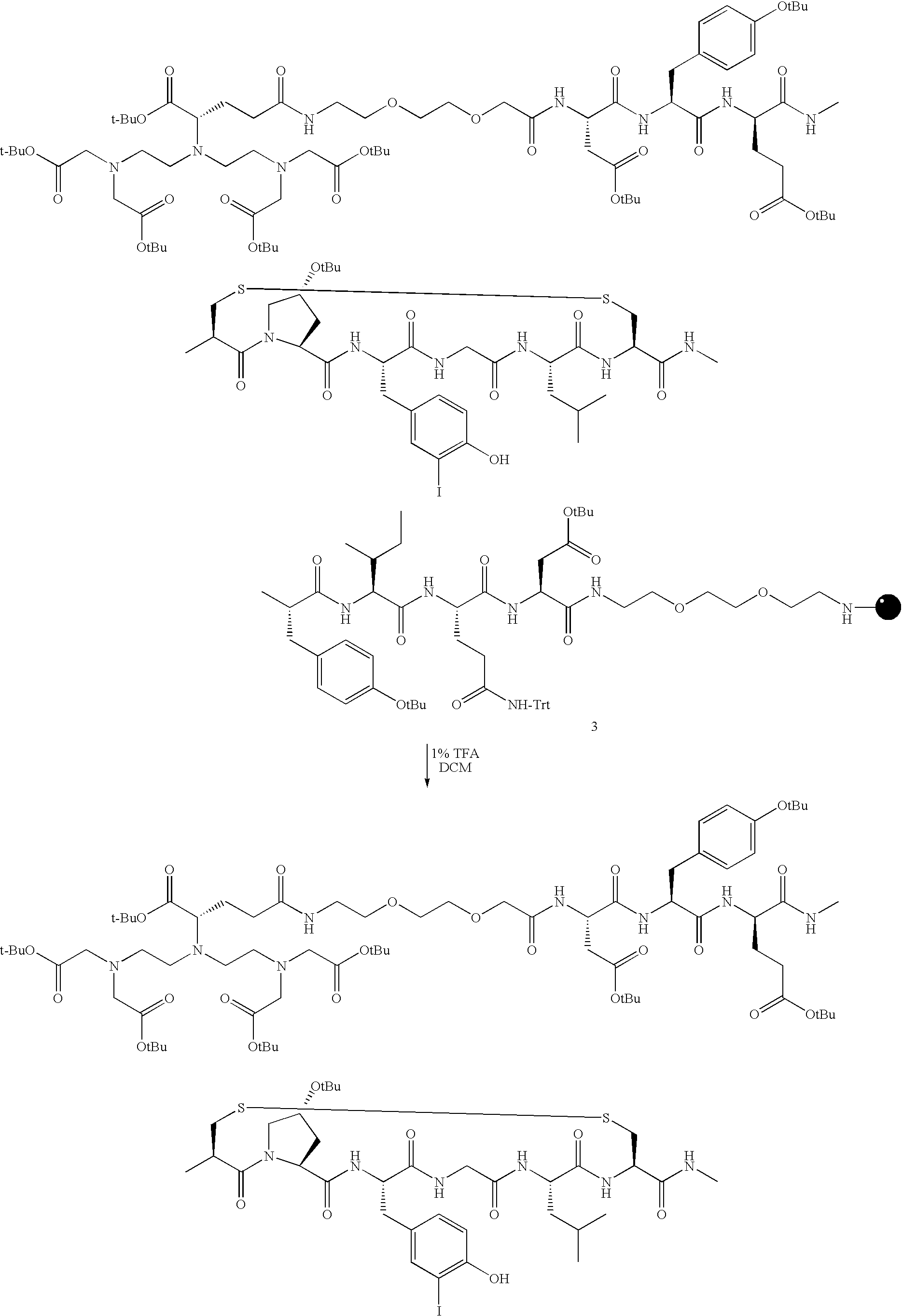 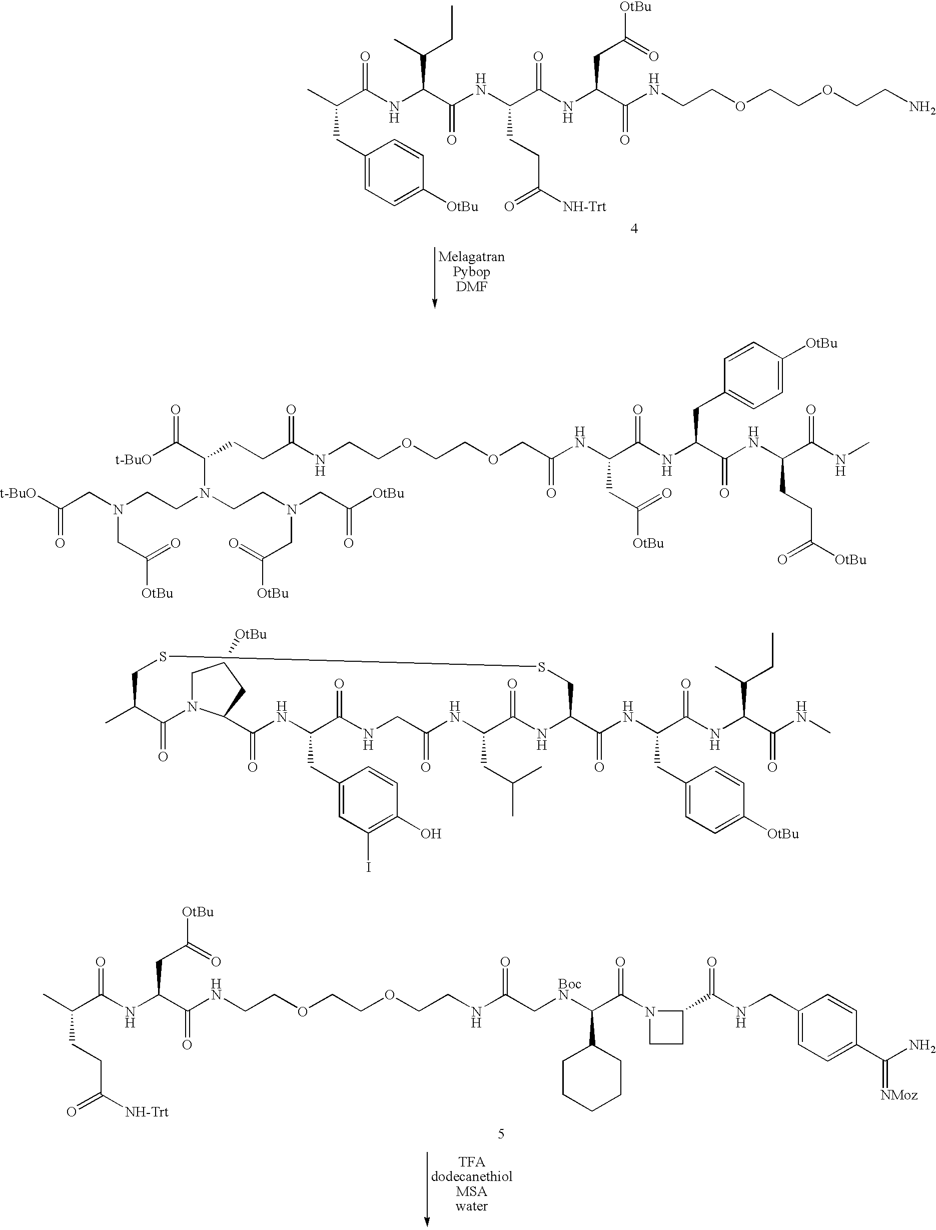 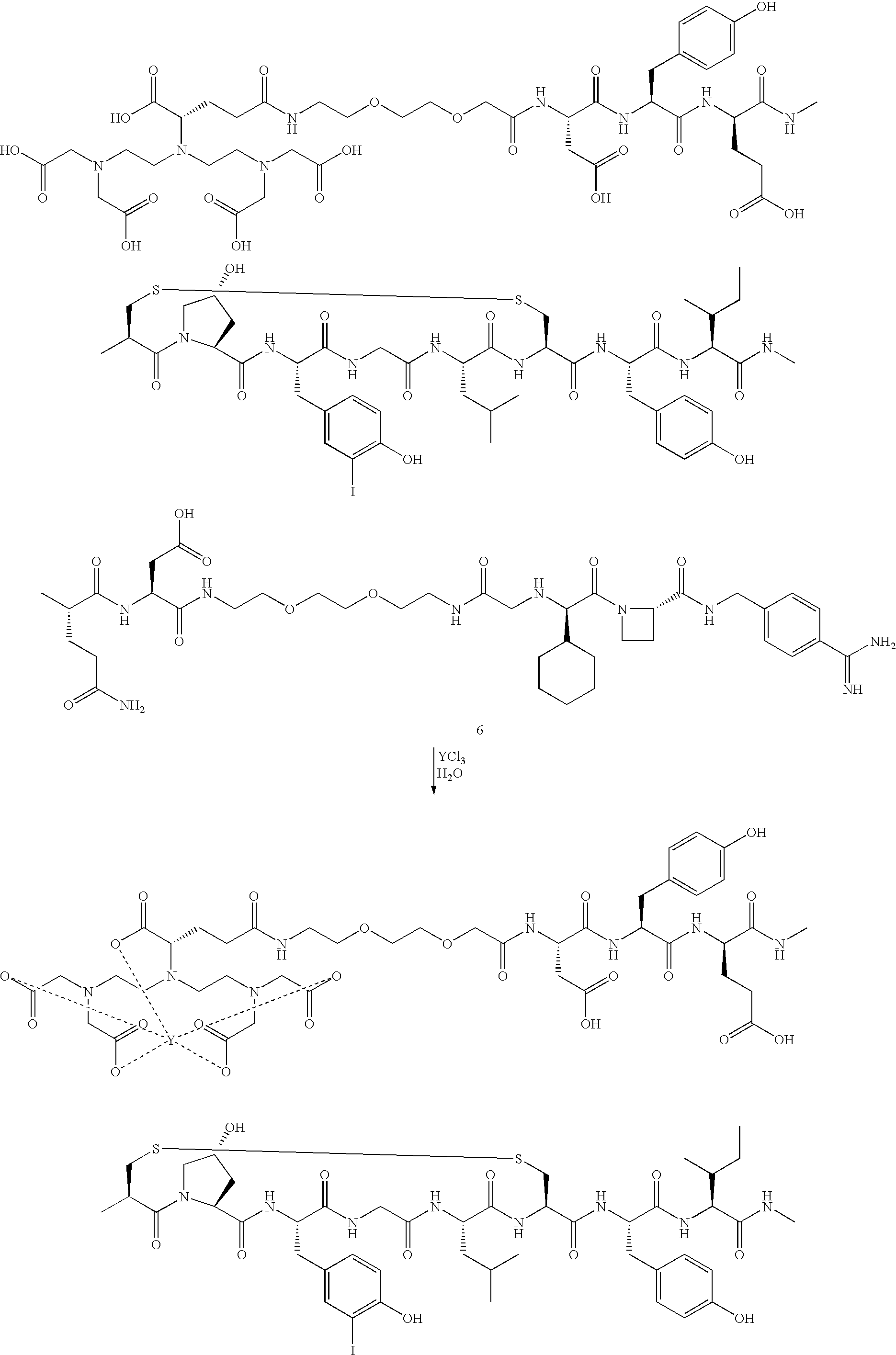 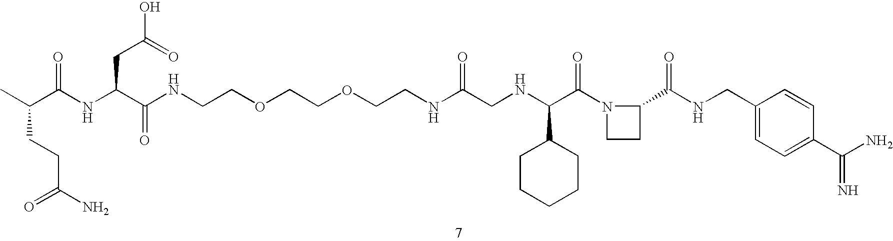
Compound 2

Suspend the peptide on resin (compound 1) in 450 mL of 5% anisole in dimethylformamide. Add thallium trifluoroacetate (1.3 g, 2.4 mmol). Shake the resin overnight for 18 hr. Wash the resin with 300 mL of DMF 2×, MeOH 1×, DCM 3×.

Compound 3

Dissolve (2.99 g, 4 mmol) of Glu-DTPE and 1 -hydroxybenzotriazole (540 mg, 4 mmol) in dimethylformamide (50 mL). Add diisopropylcarbodiimide (626 μL, 4 mmol) and 697 μL, 4 mmol) of diisopropylethylamine and shake for 2 days at room temperature. Wash the resin with 50 mL of DMF 1×, MeOH 1×, DCM 1×, MeOH 1×, DCM 2×.

Compound 4

Suspend the peptide-resin (compound 3) in 50 mL of 1 % TFA in DCM and shaken for 2 minutes. Filter the resin. Neutralize the filtrate with 10 mL of 10% pyridine in methanol. Repeat this process 20 times. Combine the neutralized filtrates and concentrate under vacuum to remove all dichloromethane. Add water (750 mL) to the concentrated solution to form a white precipitate. Filter the precipitate, wash with water, and dry to give crude compound 4. Purify the crude protected peptide by prep HPLC and lyophilize to give 370 mg of purified peptide 4.

m/e 1652.3 (M+2H/2)

Compound 5

Dissolve compound 4 (110 mg, 0.0333 mmol) in dimethylformamide (3 mL). Dissolve protected melagatran (33.1 mg, 0.0477 mmol) and pybop (24.8 mg, 0.0477 mmol) in dimethylformamide (2 mL) and add to the peptide solution. Stir the reaction for 4 hr at room temperature. Add water (40 mL) to form a precipitate. Filter the product, wash with water, and dry to give 118 mg of crude compound 5. Use without any further purification in the next step.

m/e 1327.3 (M+3H/3)

Compound 6

Dissolve compound 5 (118 mg, 0.0298 mmol) in 3 mL of 85% trifluoroacetic acid, 5% dodecanethiol, 5% methanesulfonic acid, 5% water. Stir the reaction for 1 hr at room temperature. Add ether (50 mL) to form a precipitate. Filter the precipitate, wash with ether, and dry. Dissolve the solid in 20 mL of 50:50 acetonitrile:water and lyophilize to give 94.6 mg of compound 6.

m/e 1428.8 (M+3H/3)

Compound 7

Dissolve compound 6 (94.6 mg, 0.033 mmol) in water (8 mL). Adjust the pH to 7.4 with 1 M sodium hydroxide. Add 154.8 mM YCl₃ solution (56.8 μL, 8.79 μmol) to the ligand. Adjust the pH back to 7.2 with 1 M sodium hydroxide (15 μL). Stir the reaction for 15 minutes. Check the reaction by LC/MS for completeness. Add additional YCl₃ solution (154.8 mM) (56.8 μL, 8.79 μmol) to the reaction. Adjust the pH back to 7.2 with 1 M sodium hydroxide (18 μL). Stir the reaction for 15 minutes. Check the reaction by LC/MS. Add additional YCl₃ solution (154.8 mM) (6 μL, 0.928 μmol). Isolate the product by prep HPLC and lyophilize to give 29 mg of purified product 7.

m/e 1471.7 (M+2H/2)

### Example No. 4

### Preparation of Hybrid Molecule 2 containing a Fibrin-localized Thrombin Inhibitor (Melagatran) with a Modification Group

Hybrid Molecule 2 was prepared according to the general scheme below: 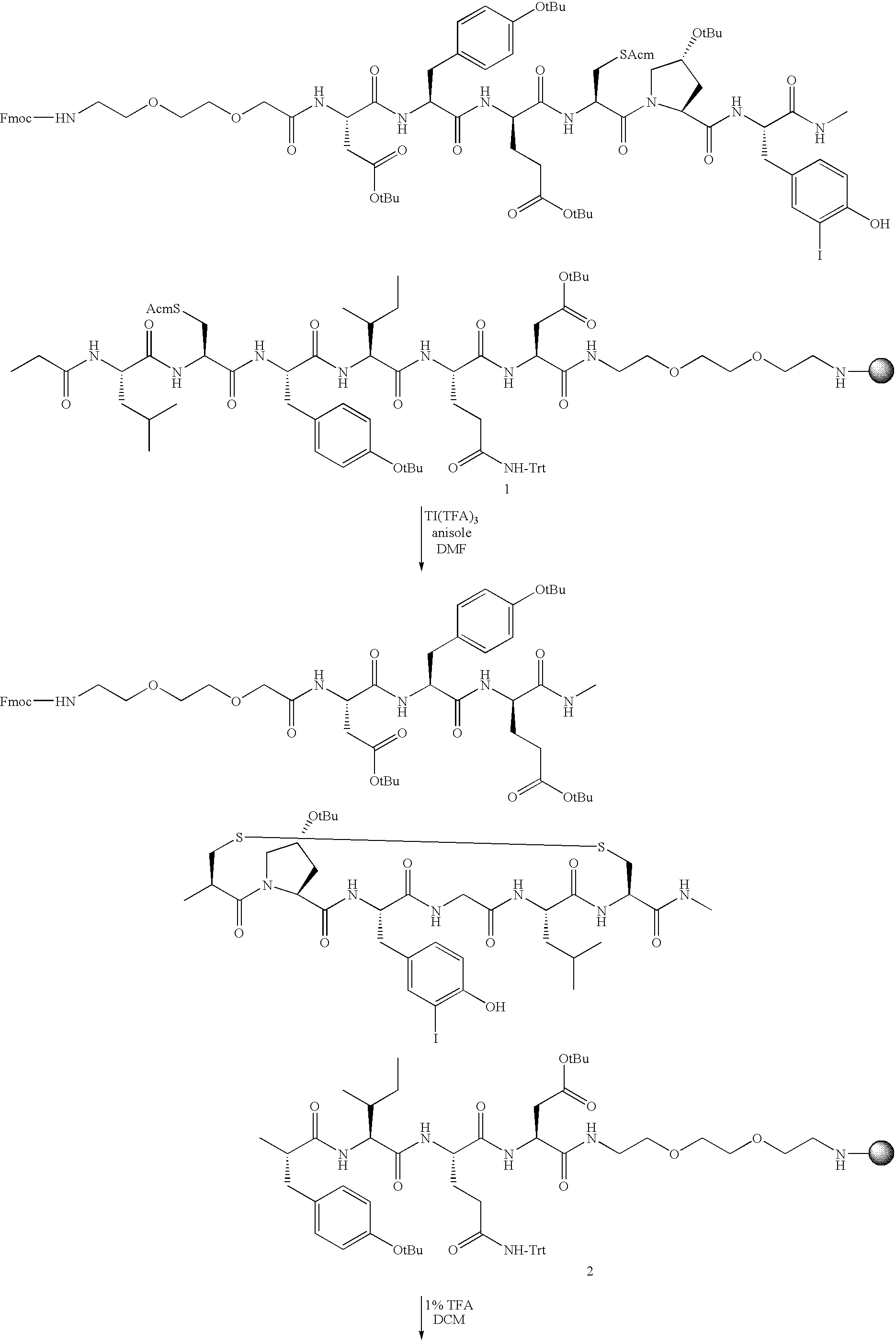 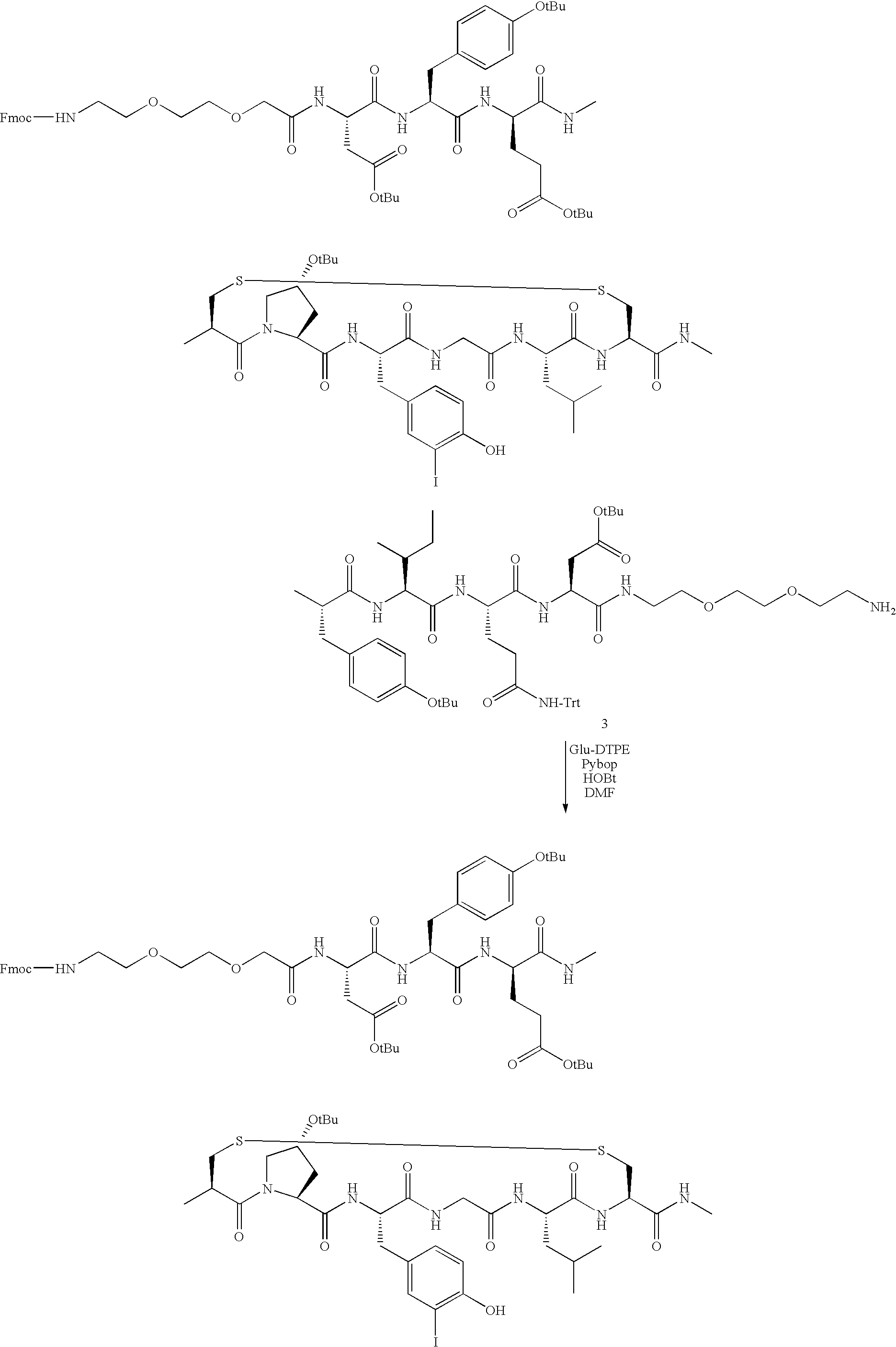 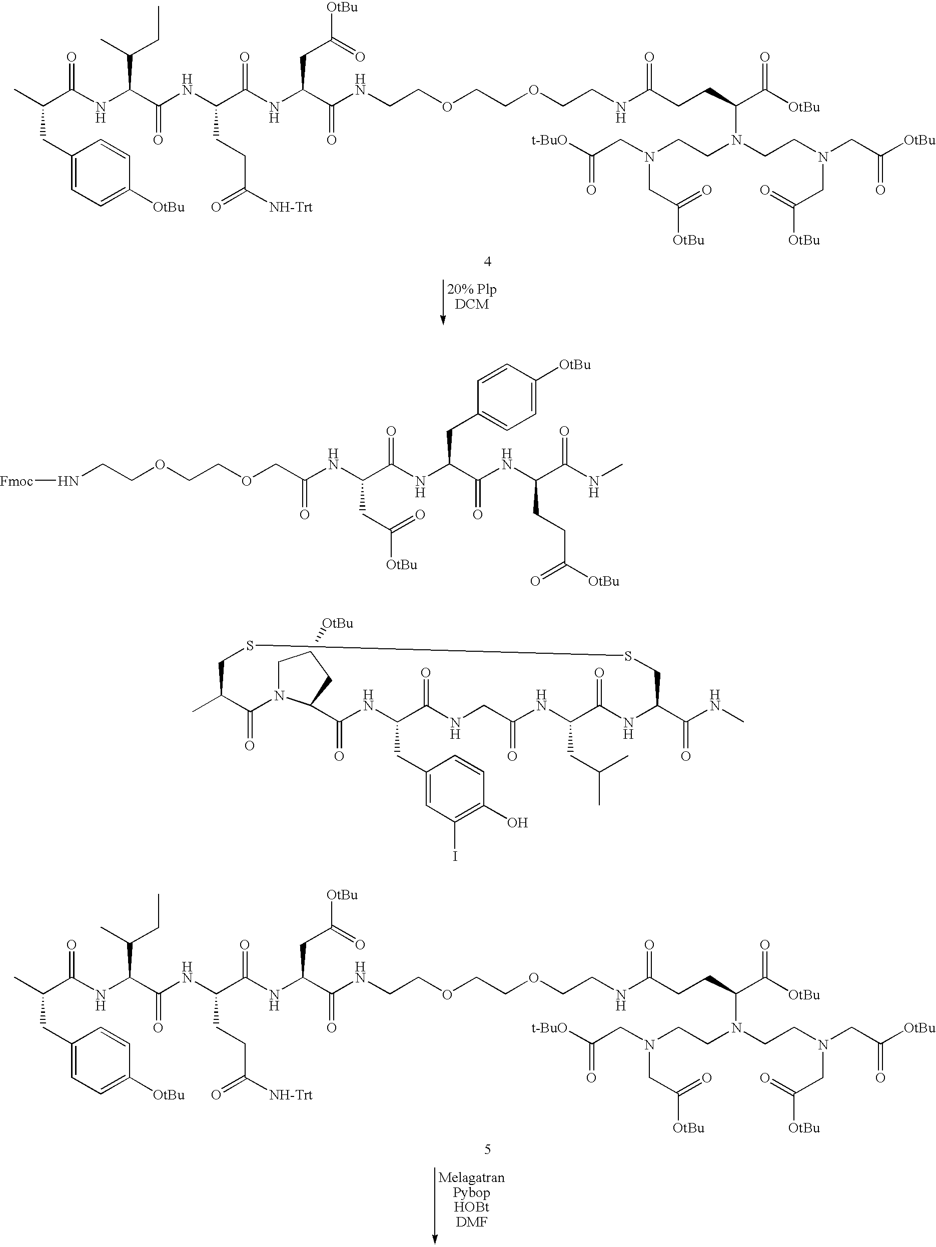 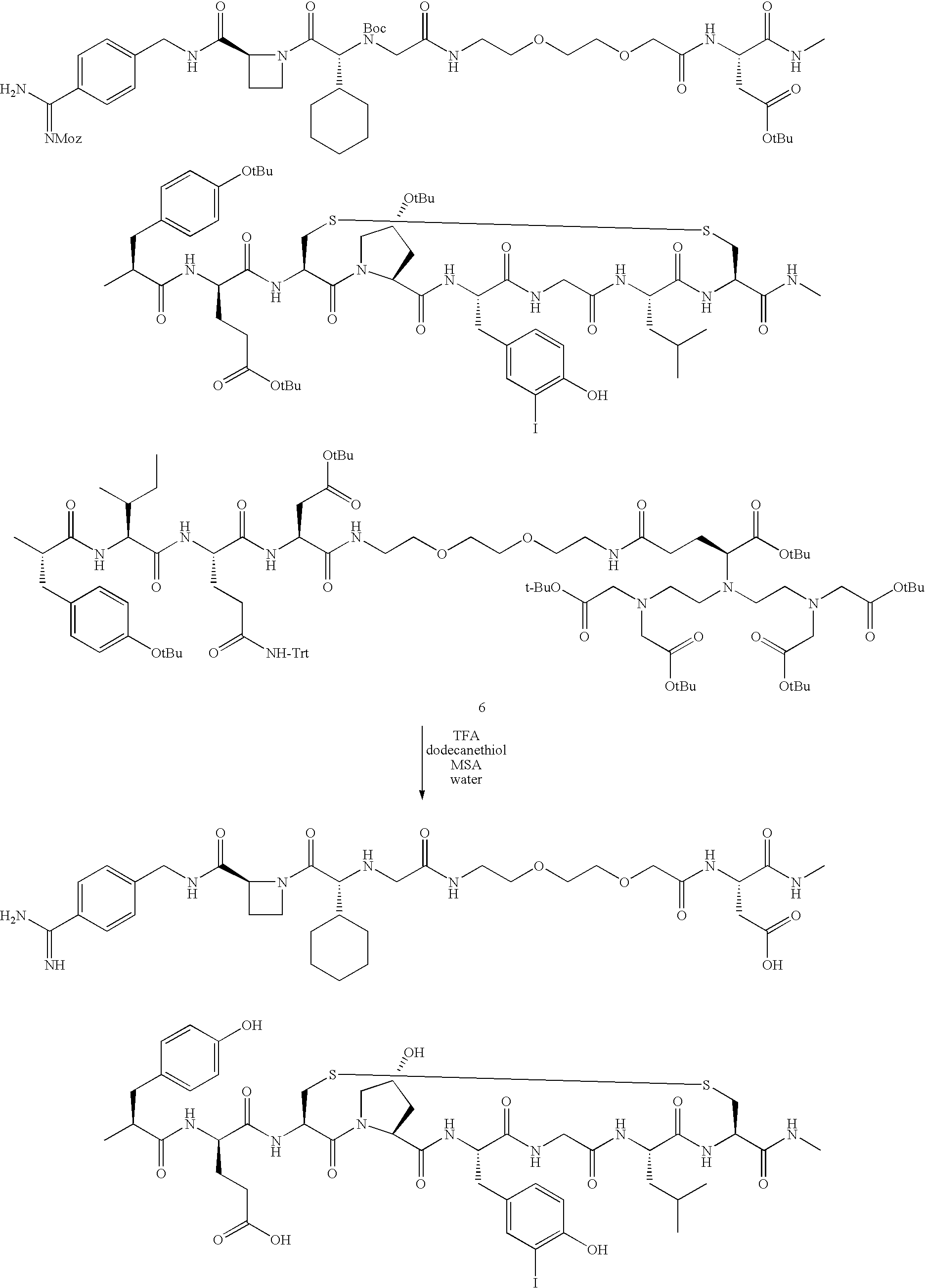 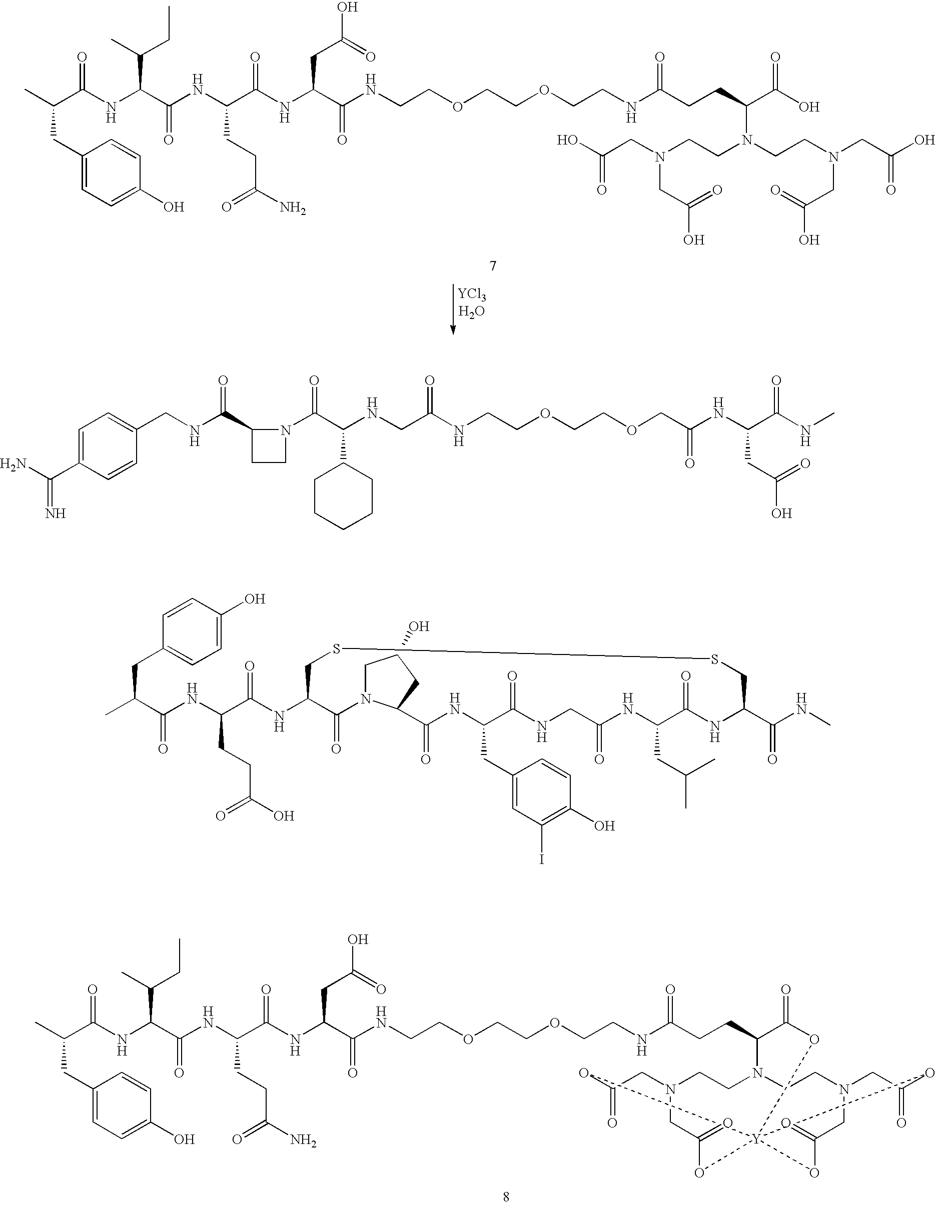
Compound 2

Suspend the peptide-resin (compound 1) in 450 mL of 5% anisol in dimethylformamide. Add thallium trifluoroacetate (1.3 g, 2.4 mmol). Shake the resin overnight for 18 hr. Wash the resin with 300 mL of DMF 2×, DCM 3×.

Compound 3

Suspend the peptide-resin (compound 2) in 100 mL of 1% TFA in DCM and shaken for 2 minutes. Filter the resin. Neutralize the filtrate with 20 mL of 2% triethylamine in methanol. Repeat this process 20 times. Combine the neutralized filtrates and concentrate under vacuum to remove all dichloromethane. Add water (500 mL) to the concentrated solution to form a precipitate. Filter the precipitate, wash with water, and dry to give crude compound 3. Purify the crude protected peptide by prep HPLC and lyophilize to give 828.6 mg of purified peptide 3.

m/e 1399.9 (M+2H/2)

Compound 4

Dissolve compound 3 (413.4mg, 0.1477 mmol) in dichloromethane (30 mL). Add Glu-DTPE (110.2 mg, 0.1477 mmol), pybop (92.2 mg, 0.1772 mmol), and 1-hydroxybenzotriazole 1 M in dichloromethane (177.2 μL, 0.1772 mmol). Add diisopropylethylamine 1 M in N-methylpyrrolidinone until the pH is 8. Stir the reaction for 20 hr at room temperature. Concentrate the reaction mixture to an oil, and purify the product by prep HPLC to give compound 4.

m/e 1763.7 (M+2H/2)

Compound 5

Dissolve compound 4 in 20% piperidine in dichloromethane (25 mL) for 30 minutes. Concentrate the reaction to an oil under vacuum. Purify the crude oil by prep HPLC to give 356.3 mg of purified compound 5.

m/e 1652.2 (M+2H/2)

Compound 6

Dissolve compound 6 (82.6 mg, 25 μmol) in dichloromethane (5.0 mL). Add 1-hydroxybenzotriazole 1M in dichloromethane (30 μL, 30 μmol). Adjust the pH to 8 with diisopropylethylamine I M in N-methylpyrrolidinone (60 μL, 60 μmol). Add melagatran (17.4 mg, 25 μmol) and pybop (15.6 mg, 30 μmol). Stir the reaction for 16 hr at room temperature. Concentrate the reaction to an oil under vacuum. Purify the oil by prep HPLC and lyophilize to give 62 mg of purified compound 6.

m/e 1990.3 (M+2H/2)

Compound 7

Dissolve compound 6 (62 mg, 15 μmol) in 5 mL of 85% trifluoroacetic acid, 5% methanesulfonic acid, 5 % dodecanethiol, and 5% water. Stir the mixture for 3 hr at room temperature. Add water (35 mL). Wash the aqueous solution with ether (2×25 mL). Lyophilize the aqueous solution. Purify the crude product by prep HPLC and lyophilize to give compound 7.

m/e 1428.8 (M+2H/2)

Compound 8

Dissolve compound 7 (15 μmol) in water (8 mL). Adjust the pH to 7 with 12 mL of 1 M sodium hydroxide and additional water (6 mL). Add YCl₃ (156.33 mM) solution (20 μL, 3.12 μmol) to the neutralized solution. Adjust the pH to 7 with 1 M sodium hydroxide (6 μL). Stir the reaction for 15 minutes. Add additional YCl₃ (156.33 mM) solution (10 μL, 1.56 μmol) to the reaction, and repeat 4 times for a total of 60 μL of YCl₃ solution. Add EDTA (100 mM) solution (1 μmol) to chelate excess metal. Isolate the product by prep HPLC to give purified compound 8.

### Example No. 5

### Preparation of Hybrid Molecule 3 containing a Fibrin-localized Thrombin Inhibitor (Melagatran) with a Modification Group

Hybrid Molecule 3 was prepared according to the general scheme below:

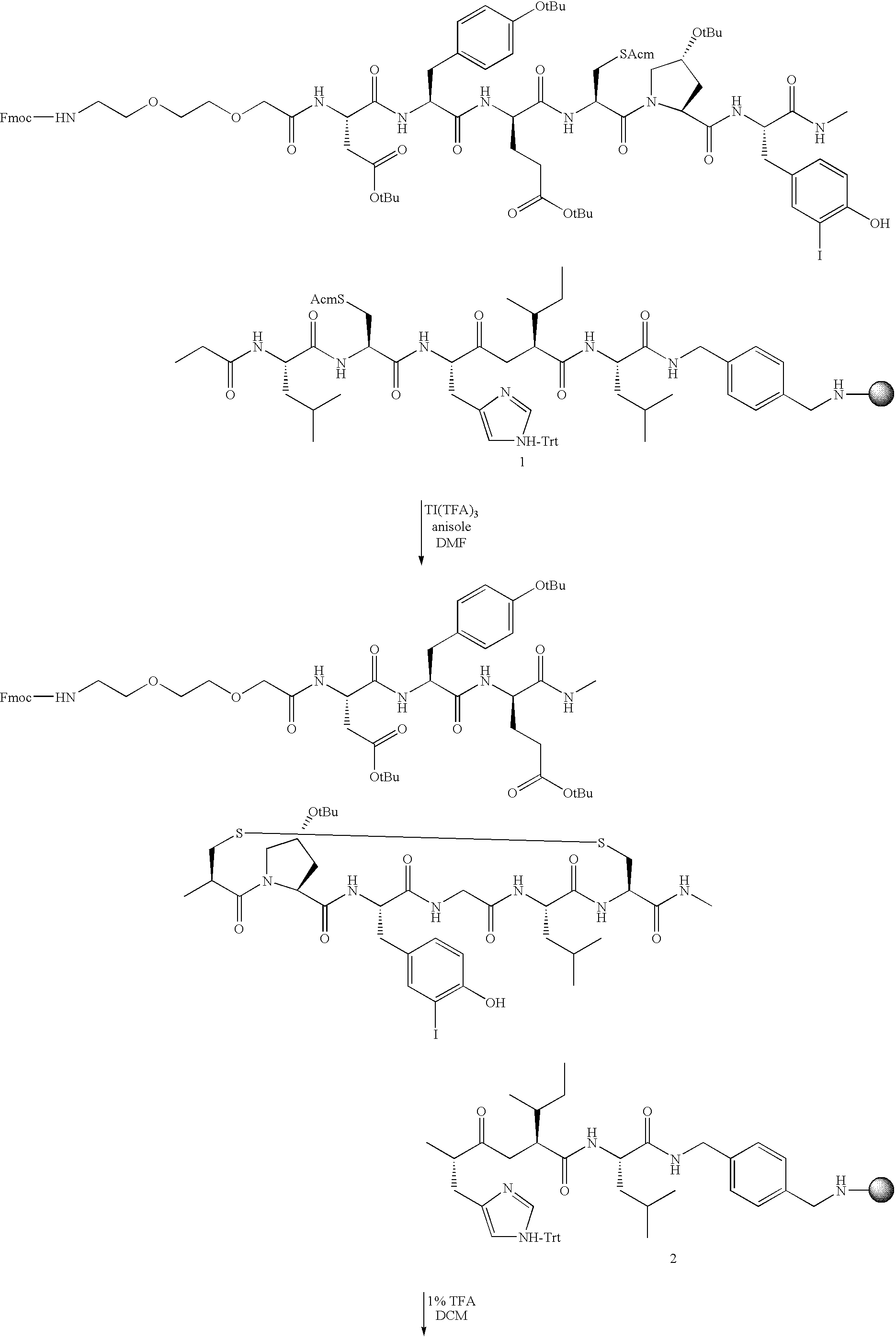

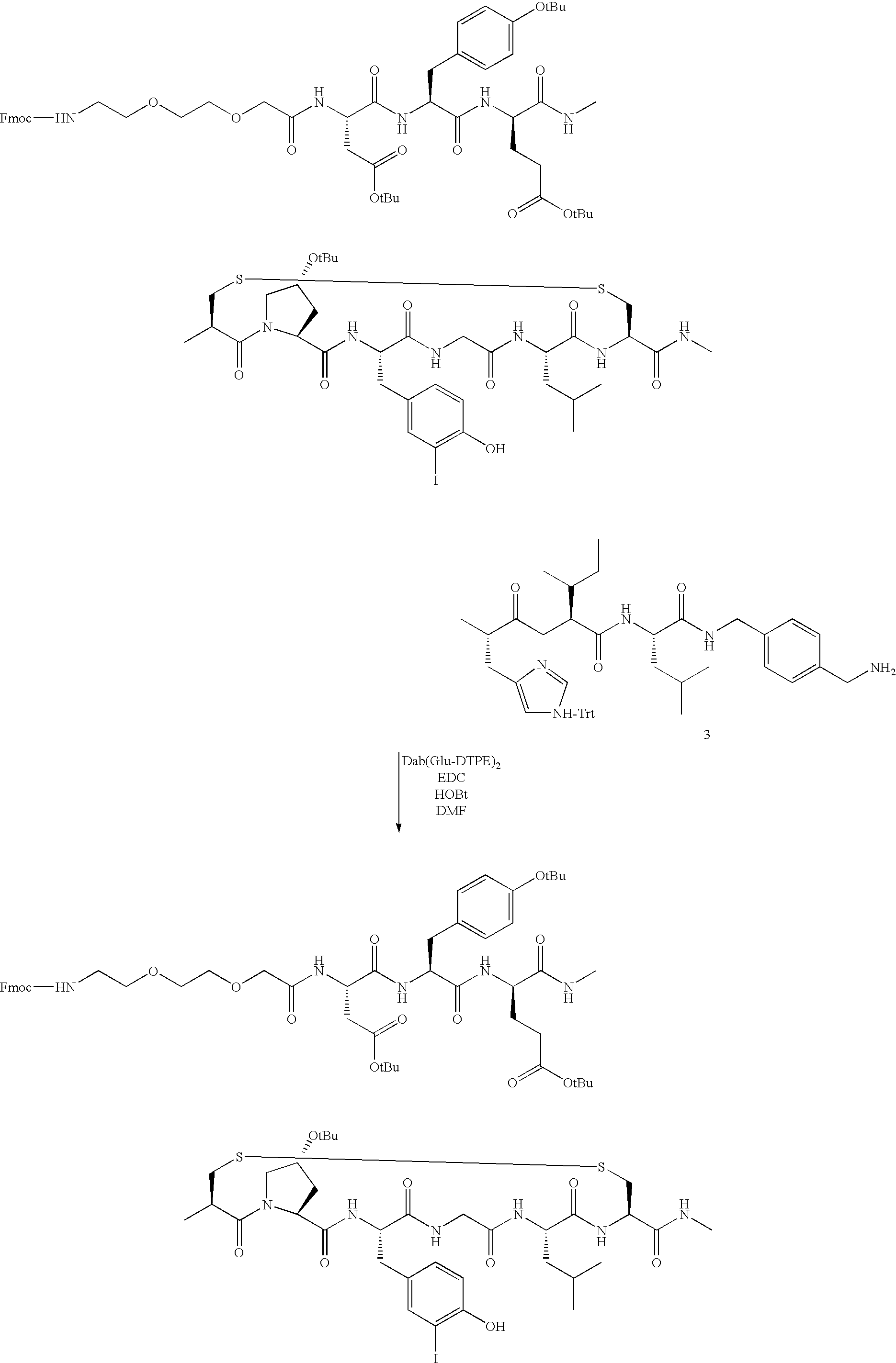

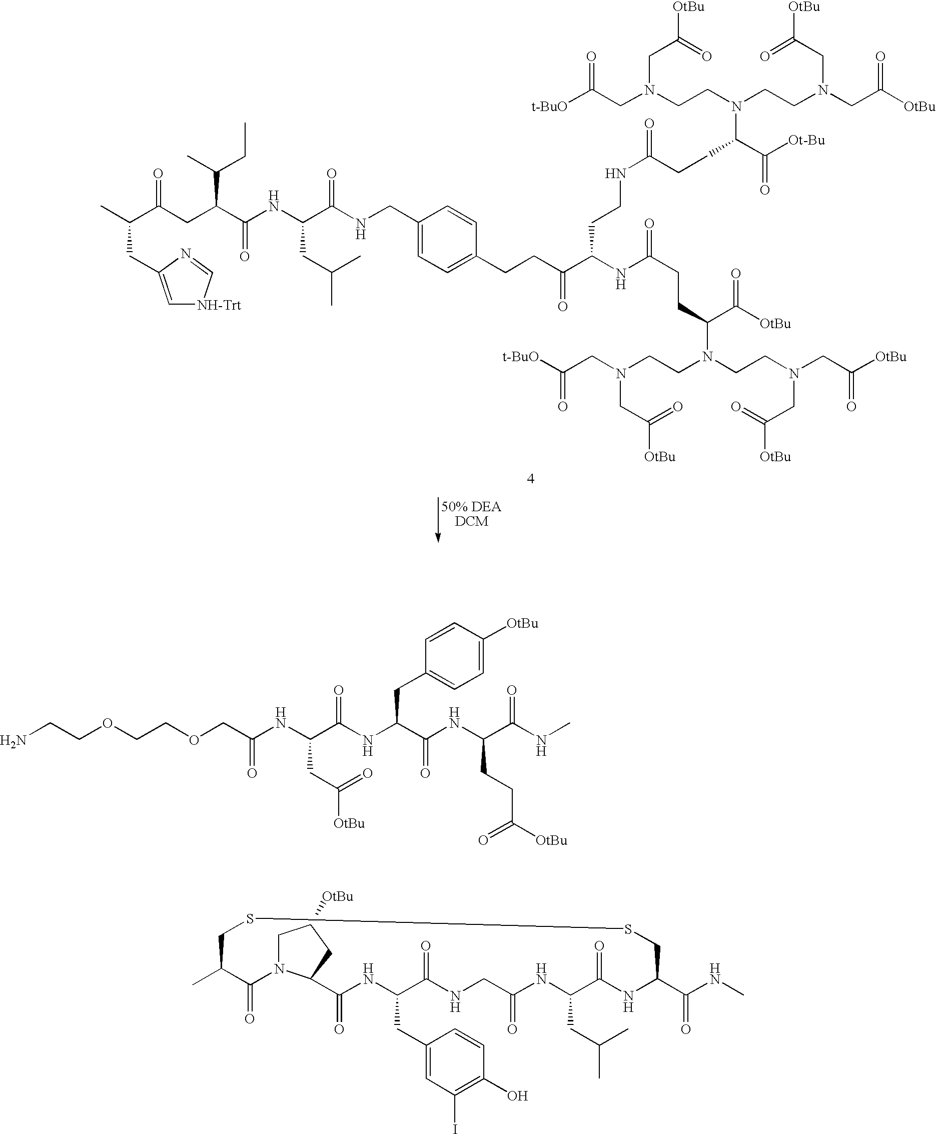

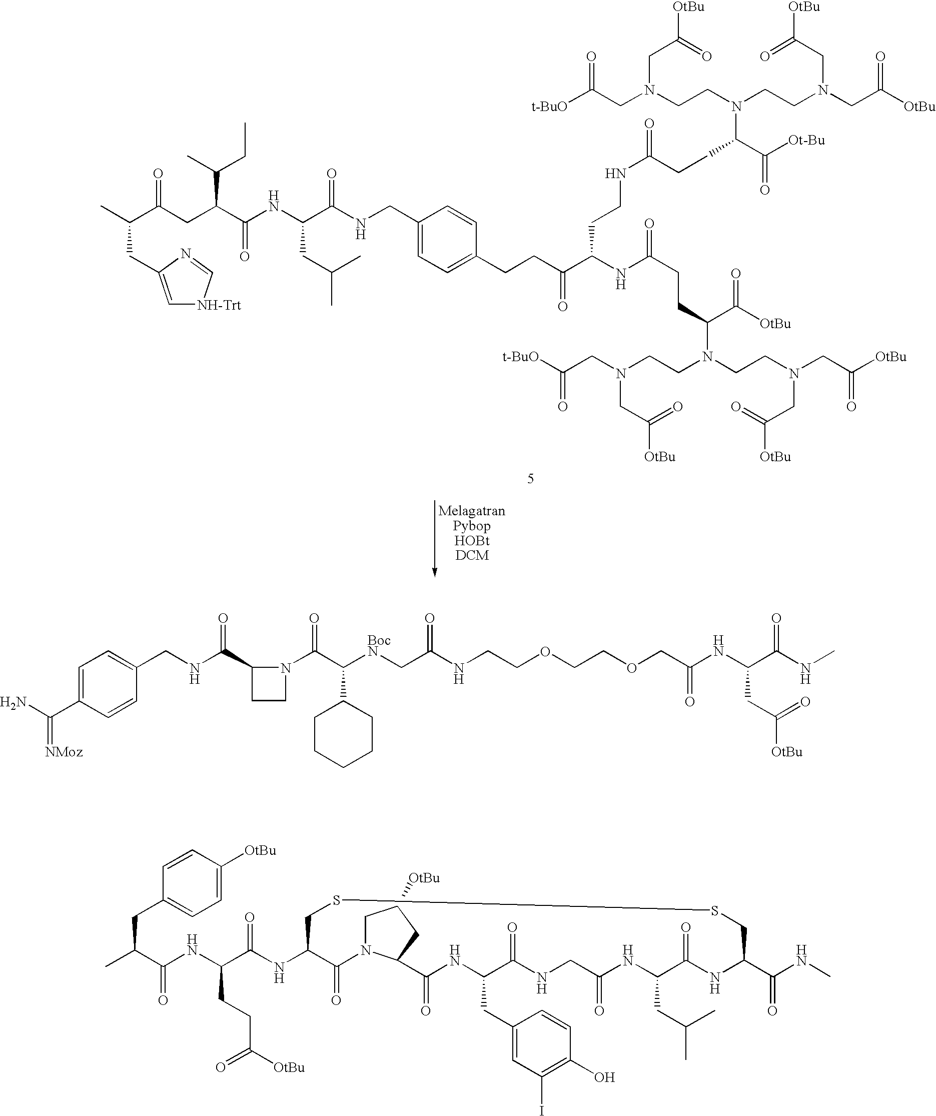

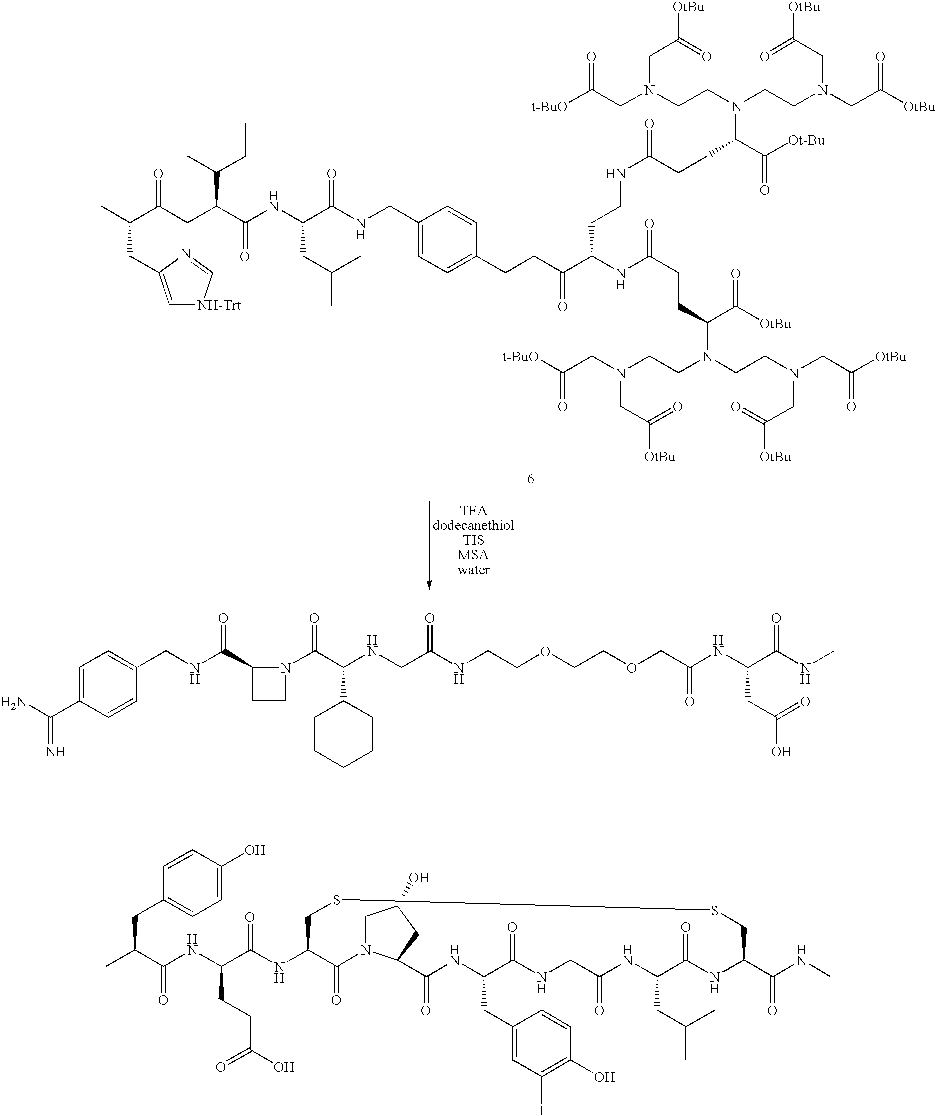

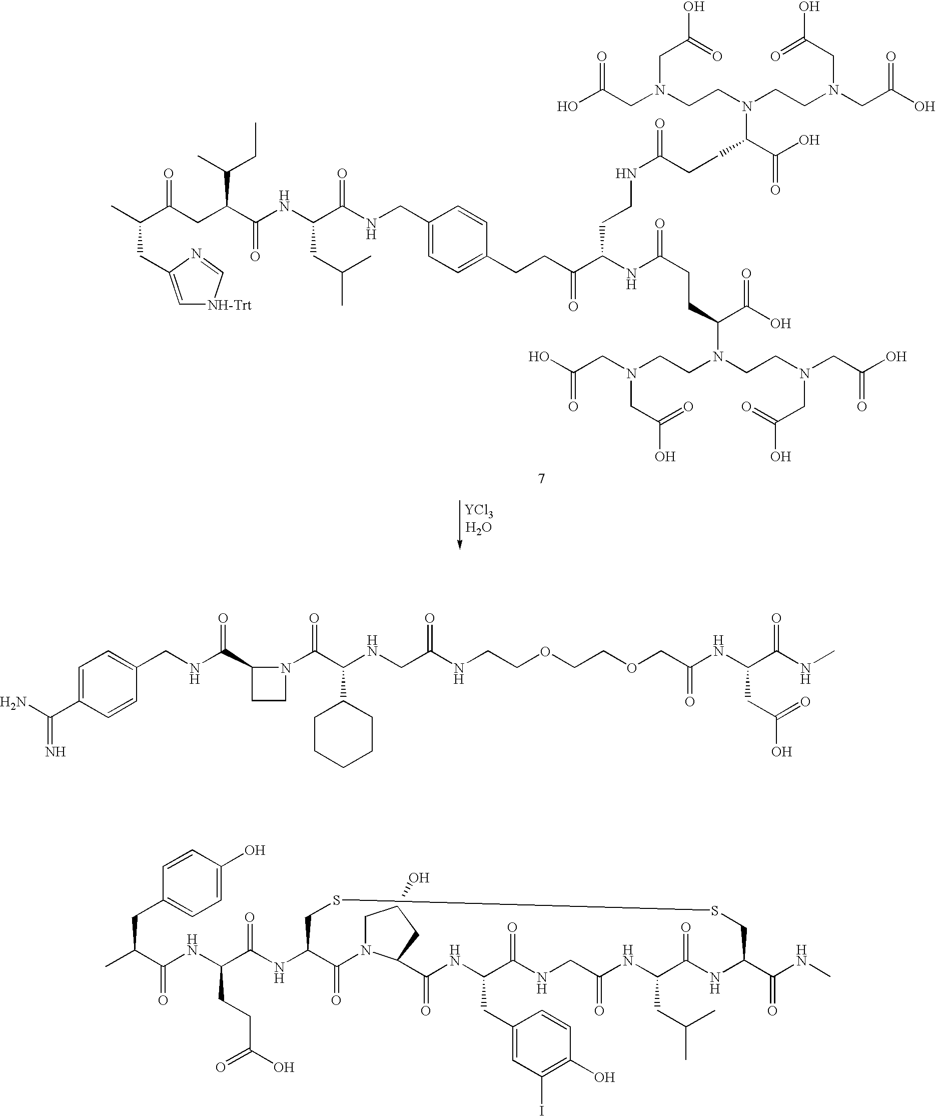

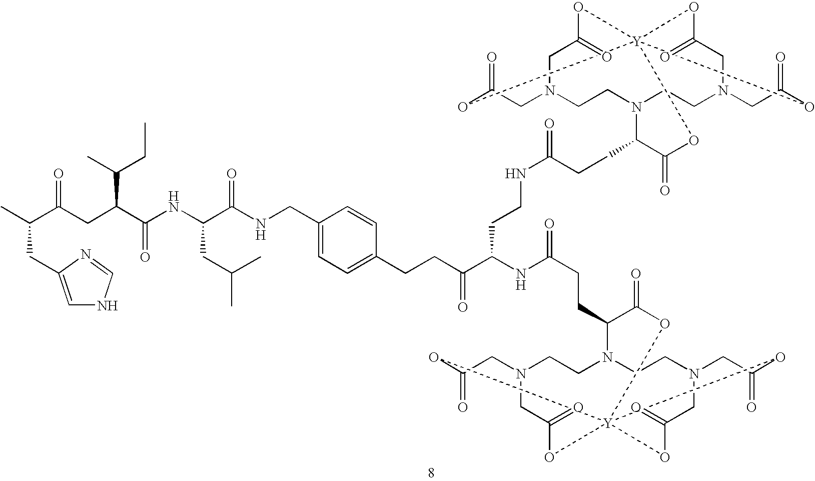

Compound 2

Suspend the peptide on resin (compound 1) in 450 mL of 5% anisole in dimethylformamide. Add thallium trifluoroacetate (1.3 g, 2.4 mmol). Shake the resin overnight for 18 hr. Wash the resin with 300 mL of DMF 2×, MeOH 1×, DCM 3×.

Compound 3

Suspend the peptide-resin (compound 2) in 100 mL of 1% TFA in DCM and shaken for 2 minutes. Filter the resin. Neutralize the filtrate with 20 mL of 2% triethylamine in methanol. Repeat this process 20 times. Combine the neutralized filtrates and concentrate under vacuum to remove all dichloromethane. Add water (500 mL) to the concentrated solution to form a precipitate. Filter the precipitate, wash with water, and drive to give crude compound 3. Purify the crude protected peptide by prep HPLC and lyophilize to give 143.2 mg of purified peptide 3.

m/e 1259.6 (M+2H/2)

Compound 4

Dissolve compound 3 (100 mg, 0.0397 mmol) in dry dichloromethane (5 mL) at 0° C. under nitrogen. Add triethylamine (10 μL, 0.079 mmol). Dissolve Dab(Glu-DTPE)₂ (62.5 mg, 0.0397 mmol) in dry dichloromethane at 0° C. Add EDC (8 mg, 0.0417 mmol) and 1-hydroxybenzotriazole (5.7 mg, 0.0471 mmol) and pour the solution into the starting peptide solution. Add triethylamine (10 μL, 0.079 mmol) again, and allow the reaction to come to room temperature. Stir the reaction at room temperature for 24 hr. Concentrate the reaction to a residue under vacuum. Purify the crude product by prep HPLC and lyophilize to give 90 mg of purified compound 4.

m/e 2037.7 (M+2H/2)

Compound 5

Dissolve compound 4 (90 mg, 0.0221 mmol) in 50% diethylamine in dichloromethane (4 mL). Stir the reaction for 2 hr at room temperature. Concentrate the reaction to a residue under vacuum. Purify the crude product by prep HPLC to give 73 mg of purified compound 5.

m/e 1926.2 (M+2H/2)

Compound 6

Dissolve compound 6 (73 mg, 18.95 μmol) in dry dichloromethane (5.0 mL) at 0° C. under nitrogen. Add diisopropylethylamine (7 μL, 38 μmol). Dissolve melagatran (13.1 mg, 18.95 μmol), pybop (11.8 mg, 22.74 μmol) and 1-hydroxybenzotriazole (3.1 mg, 22.74 μmol) in dry dichloromethane (4 mL). Combine the solutions and stir the reaction at 0° C. Add more diisopropylethylamine (3.5 μL, 18.95 μmol) and allow the reaction to warm to room temperature. Stir the reaction for 6 hr at room temperature. Concentrate the reaction to a residue under vacuum. Purify the residue by prep HPLC and lyophilize to give 68 mg of purified compound 6.

m/e 1059.8 (M+3H/3)

Compound 7

Dissolve compound 6 (68 mg, 15 μmol) in 6 mL of 85% trifluoroacetic acid, 2.5% methanesulfonic acid, 2.5% triisopropylsilane, 5 % dodecanethiol, and 5% water. Stir the mixture for 3 hr at room temperature. Add cold ether to precipitate the product. Filter the product, wash the product with cold ether and dry to give 40 mg of compound 7.

m/e 1079.3 (M+3H/3)

Compound 8

Dissolve compound 7 (40 mg, 12 μmol) in water (8 mL). Adjust the pH to 6.5 with 300 μL of 1 M sodium hydroxide. Add YCl₃ (156.33 mM) solution (20 μL, 3.12 μmol) to the reaction. Adjust the pH to 7 with 1 M sodium hydroxide. Stir the reaction for 10 minutes. Repeat this process 2 more times. Check the reaction by LC/MS. Add additional YCl₃ (156.33 mM) solution (20 μL, 3.12 μmol) to the reaction. Adjust the pH to 7 with 1 M sodium hydroxide. Stir the reaction for 10 minutes. Repeat this process 2 more times. Check the reaction by LC/MS. Add additional YCl₃ (156.33 mM) solution (10 μL, 1.56 μmol) to the reaction. Adjust the pH to 7 with 1 M sodium hydroxide. Isolate the product from solution by prep HPLC to give 37.2 mg of purified compound 8.

### Example No. 6

### Preparation of Fibrin-localized Factor Xa Inhibitors

The schemes below demonstrate the preparation of fibrin-localized Factor Xa inhibitors.

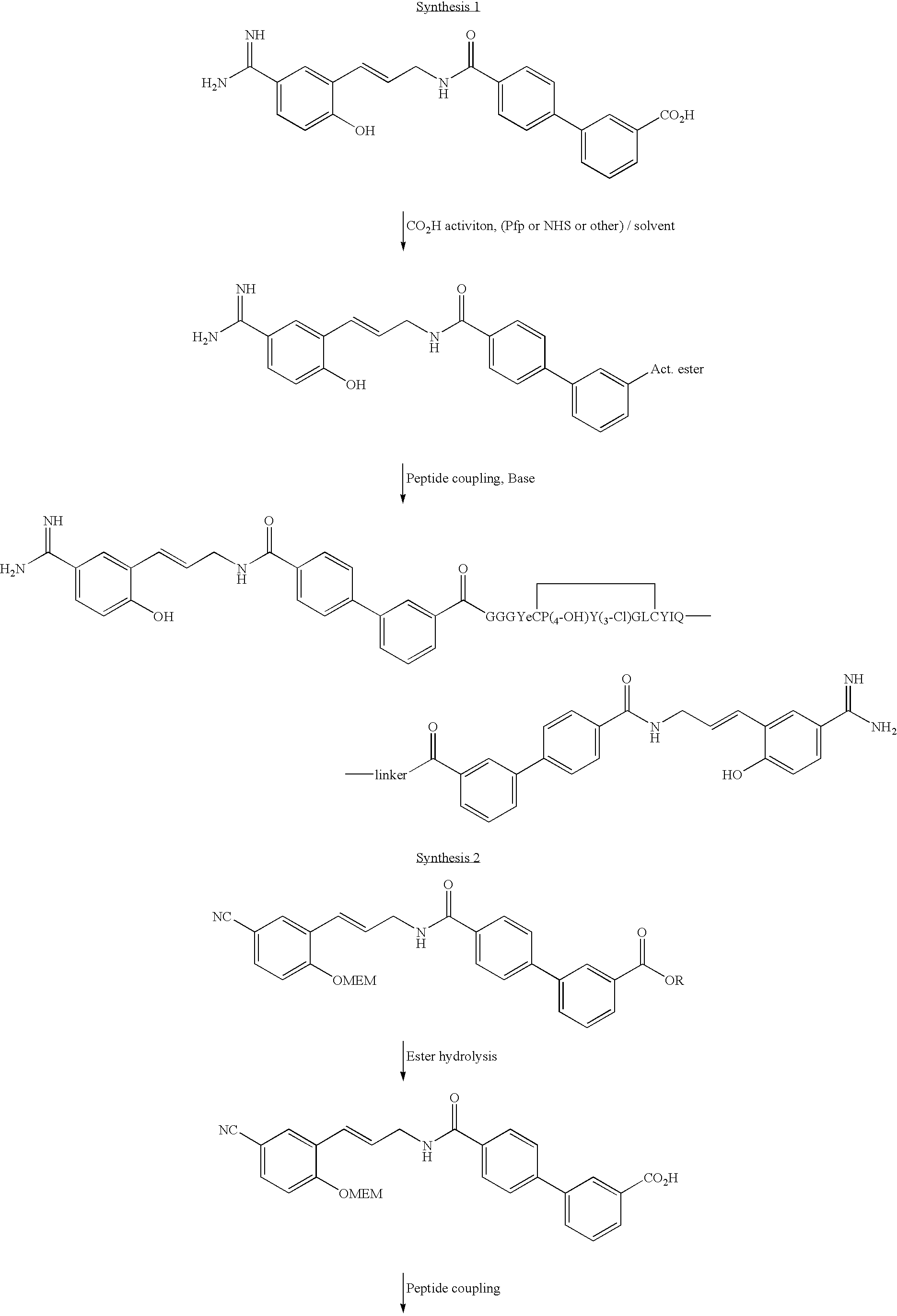

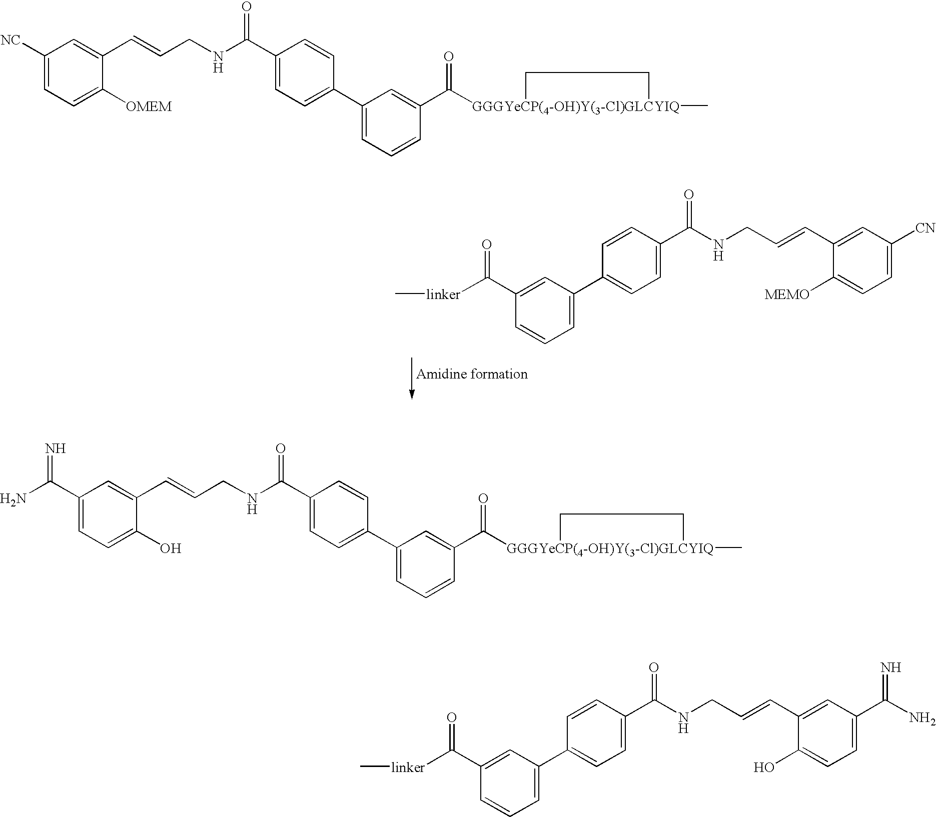

### Example No. 7

### Preparation of a Fibrin-targeted TAFIα Inhibitor

The following scheme demonstrates the preparation of a fibrin-targeted TAFIα Inhibitor.

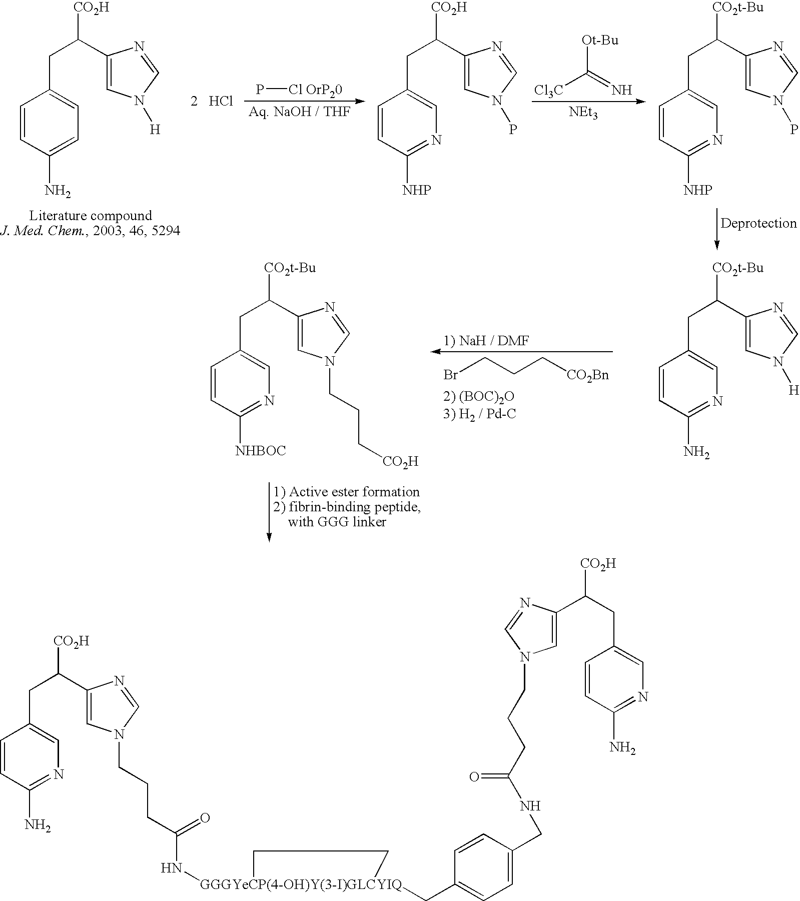

### Example No. 8

### Preparation of a Fibrin-targeted Thrombin Inhibitor

The following scheme demonstrates the preparation of a fibrin-targeted thrombin inhibitor.

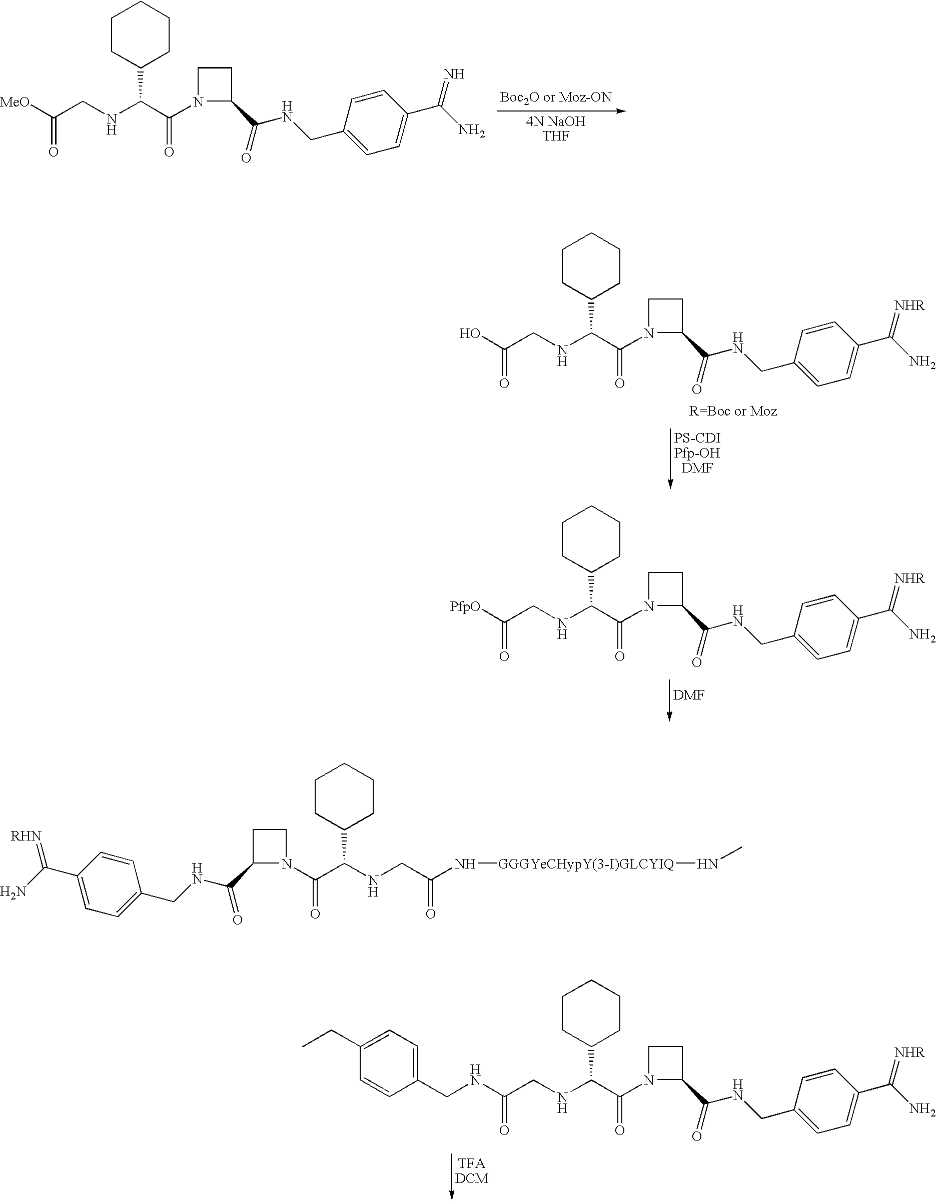

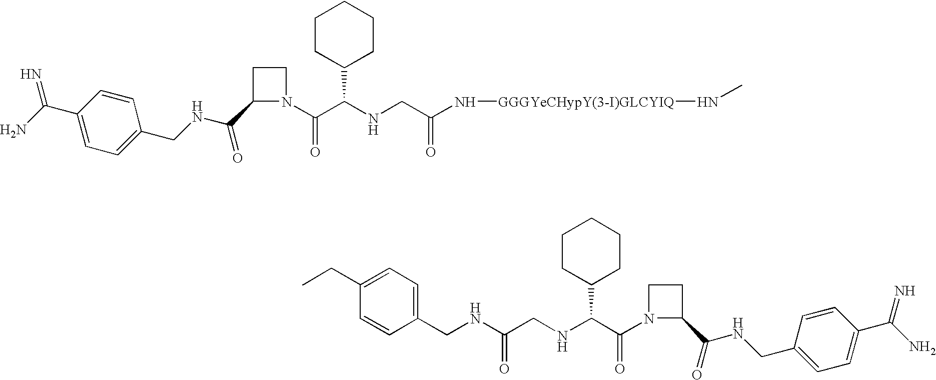

### Example No. 9

### Preparation of a Fibrin Targeted Antiplatelet Conjugate

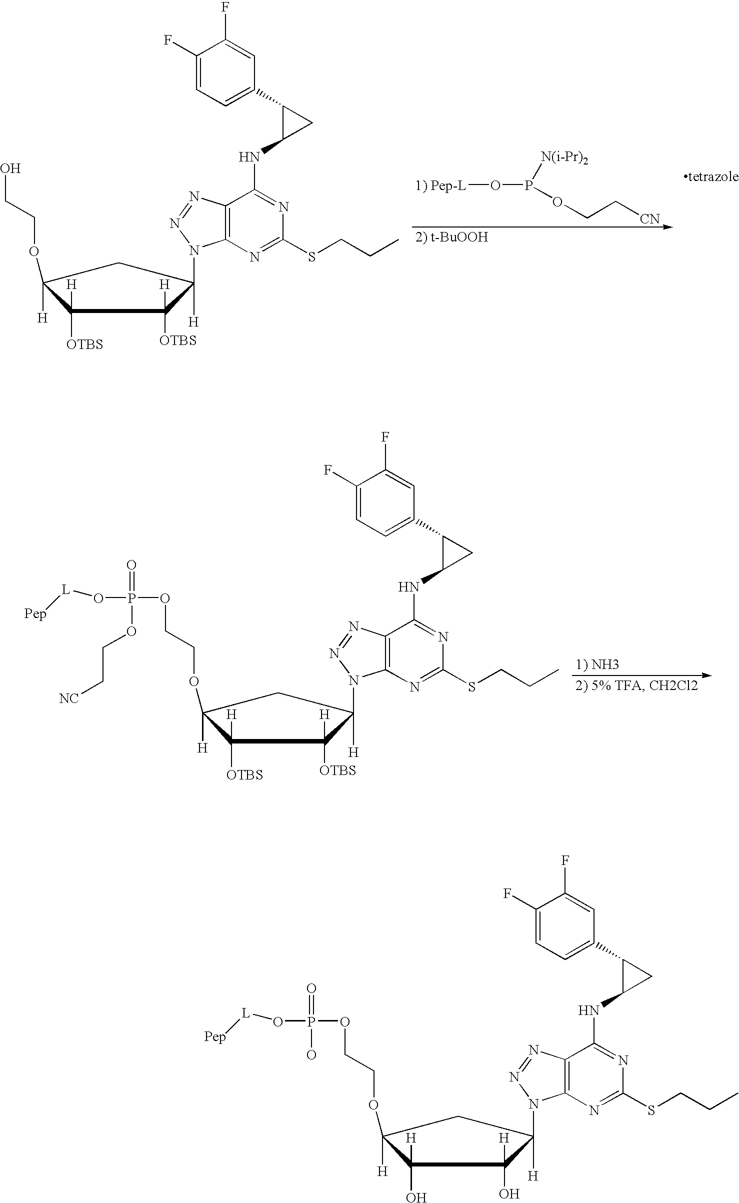

### Example No. 10

### Measurement of Target Inhibition and Protease Selectivity by Fibrin-targeted Inhibitors

Inhibition constants (K₁) for test compounds against haemostatic enzymes and trypsin are measured using enzyme-specific substrates under optimal buffer conditions for a given assay (Table 1). The typical substrate for these assays is a modified polypeptide, resembling a fragment of the natural substrate for a given enzyme, fused to a chromogenic or fluorogenic label that is cleaved in the course of reaction. The common labels are para-nitroanilin (pNA) derivatives, 7-amino-4-methylcoumarin (AMC) or other organic fragments displaying similar properties. Enzyme catalyze cleavage of peptide-label chemical bond releases free chromophore or fluorophore, hence increasing the UV absorbance or fluorescence in the assay solution. The rate of absorbance or fluorescence increase is directly proportional to the concentration of free (uninhibited) enzyme in the assay.

TABLE 1

| Enzyme | Substrate ^{a)} | Structure | Buffer |
|---|---|---|---|
| Thrombin | Spectrozyme TH (238) | H-D-HHT-Ala- | 50 mM HEPES, 150 mM |
| | | Arg-pNA | NaCl, pH 7.4 |
| | Spectrozyme TH | H-D-CHA-Ala- | 50 mM HEPES, 150 mM |
| | (238XF) | Arg-AMC | NaCl, pH 7.4 |
| Trypsin | Spectrozyme TRY | Cbo-Gly-D-Ala- | 50 mM Tris, 150 mM NaCl, |
| | (216) | Arg-pNA | pH 8.4 |
| FXa | Spectrofluor FXa | CH₃SO₂-D-CHA- | 50 mM Tris, 150 mM NaCl, |
| | (222XF) | Gly-Arg-AMC | 5 mM CaCl₂, pH 8.0 |
| uPA | Spectrozyme UK | Bz-β-Ala-Gly-Arg- | 50 mM Tris, 150 mM NaCl, |
| | | AMC | pH 8.8 |
| Plasmin | Spectrozyme PL | H-D-Nle-HHT- | 50 mM Tris, 150 mM NaCl, |
| | | Lys-pNA | pH 7.4 |
| tPA | Spectrofluor tPA | CH₃SO₂-D-Phe- | 50 mM Tris, 150 mM NaCl, |
| | (444F) | Gly-Arg-AMC | pH 8.0 |
| FXVIIa | Spectrozyme FVIIa | CH₃SO₂-D-CHA- | 100 mM Tris, 100 mM NaCl, |
| | (217) | But-Arg-pNA | 5 mM CaCl₂, pH 8.4 |
| TF | Actichrome TF (846) ^{b)} | | |

^{a)} American Diagnostica catalog numbers provided
^{b)} Inhibition of TF is evaluated by the Actichrome TF kit. The kit contains conjugated system of FVIIa, FX and Spectrozyme FXa substrate which is complimentary to the TF activity. To deconvolute potential inhibition of TF, inhibition of FVIIa and FXa should be initially established.

A typical assay is performed in a polystyrene microtiter plate using an array where inhibitor and substrate concentration are coordinately varied, as follows. A chromogenic or fluorogenic substrate is added in the appropriate assay buffer at a fixed concentration in the wells of a microtiter plate. Three or more sets of samples are set up, each at a different concentration of substrate; three replicates of each set are tested. The enzyme inhibition assay is conducted at final concentrations of the substrate close to or above the K_{M} value for the assayed enzyme. For each set of samples at fixed substrate concentrations, the inhibitor to be tested is added at different concentrations, including a negative (no inhibitor) control, into the wells. The actual range is typically around one-tenth to ten times the suspected K₁. The reaction is started by addition of an enzyme stock solution to each well of the plate. The change in absorbance or fluorescence is recorded on a plate reader as function of time. Initial rates of substrate turnover are calculated from the linear portions of progressive kinetic curves by the least squares fitting method. The value for initial rates are fitted to the following equation, describing the competitive inhibition using suitable nonlinear regression software:

$$v = \frac{{V}_{max}}{1 + \frac{{K}_{M}}{[ S ]} ( 1 + \frac{[ I ]}{{K}_{I}} )} , . \qquad Equation 1$$

Where ν is an initial rate, Vₘₐₓ and K_{M} are Michaelis-Menten constants, [S] and [1] are substrate and inhibitor concentrations and K, is the inhibition constant.

Bisconjugate Results

Spectrozyme TH solutions of 100, 50 or 25 μM concentrations (10-fold of final concentration of substrate (Example 2 Bisconjugate) in assay) were added in 10 μL aliquots to each well of a black-masked, clear-bottom polystyrene 96-well microplate. 65 μL of different Example 2 Bisconjugate stock dilutions in assay buffer were added in multiple replicates (n=3-5) to each well of the microplate to produce 0 to 100 nM final concentration of inhibitor in assay. The assay was initiated by addition of 25 μL of thrombin working stock solution (0.1 nM final enzyme concentration) to all wells (final volume, 100 μL). The change in fluorescence associated with turnover of the substrate by the remaining thrombin activity was recorded using 360 nm excitation and 440 nm emission filters. The reaction rate was calculated from the first 10 min of reaction. Resulting rates for all samples were fitted in the equation for competitive inhibition. The average K₁ was determined to be 0.37±0.04 nM.

### Example No. 11

### In Vivo Efficacy of Fibrin-targeted Inhibitors: Arterial Thrombosis Model

Rabbits are initially sedated with a subcutaneous injection of Xylazine (5 mg/kg). Approximately 5 min following sedation, the animals are anesthetized with a dose of Ketamine (50 mg/kg) and Acepromazine (2.5 mg/kg) intramuscularly to achieve the appropriate level of anesthesia. Anesthetic depth will be assessed using the pain withdraw reflexes and EKG. Anesthesia is given intravenously every 45-60 min at 5-10 mg/kg to maintain proper anesthetic depth.

Left carotid artery is isolated using a ventral midline approach. A stenosis is initiated on the carotid artery reducing blood flow by 70-80%. 1 cm segments of carotid artery are placed in a plastic tube opened lengthwise, together with a filter paper (diameter 3 mm) soaked in ferric chloride (FeCl₃, 40 μL, 40% w/w). After 15-30 min exposure of the artery to FeCl₃, the plastic tube with the filter paper is removed and the artery is rinsed with saline. The time to vessel occlusion is monitored using a Doppler flow probe, which is attached to the carotid artery. ¹²⁵I fibrinogen (30 μCi/kg) is administered 5 min before initiation of the stenosis. The saline, test or control compounds are given via the jugular vein as an intravenous bolus injection immediately before or at the end of FeCl₃ exposure. Blood sample are collected at 1, 3, 5, 12, 20, 30, 45, 60 and 90 min after compound injection via the contra lateral jugular vein. The time that blood flow dropped to zero is recorded as the occlusion time of blood vessel. At the end of experiment, animals are sacrificed with an overdose of pentobarbital (120 mg/kg). Both the clot from the FeCl₃ damaged artery, and the damaged artery after clot removal are weighed. Right carotid artery is used as a control. Concentration of test compound and fibrin in the clot are detected by γ-countering.

Pharmacokinetics of compound are analyzed by using the WinNonLin program.

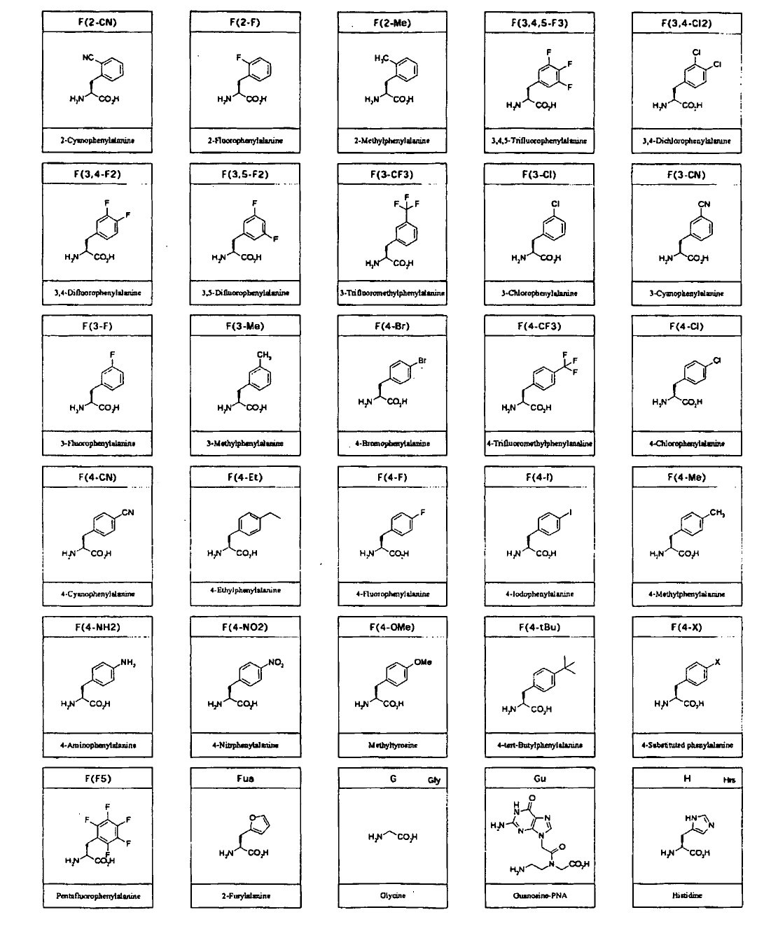

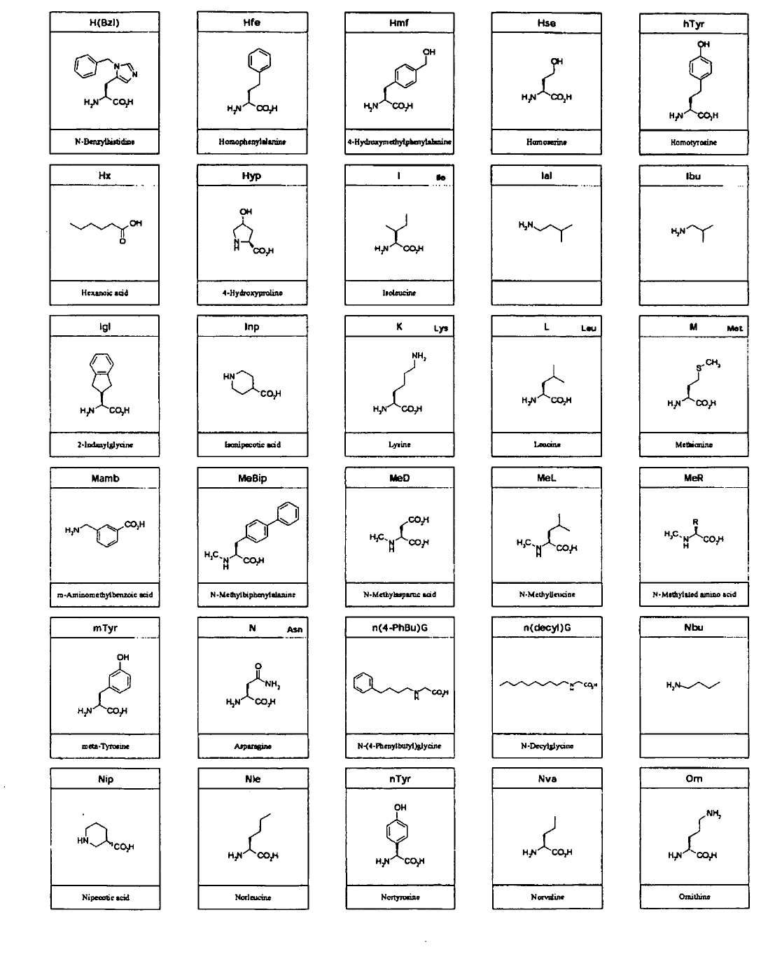

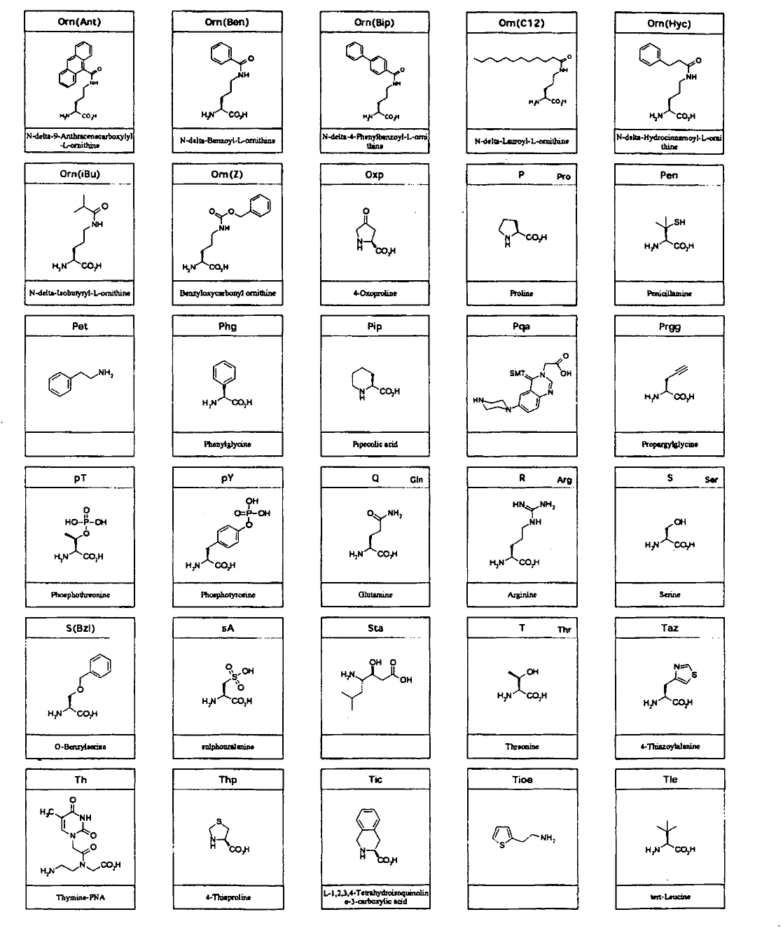

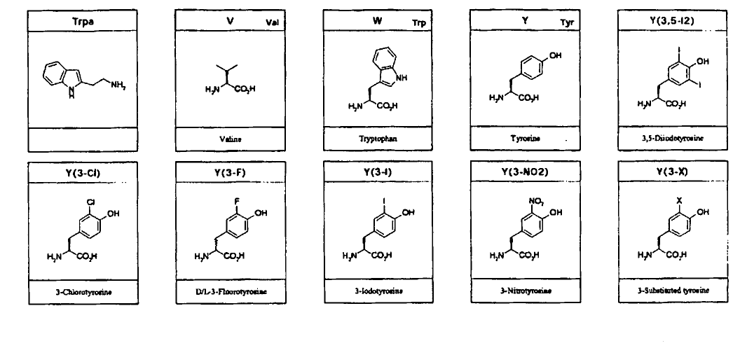

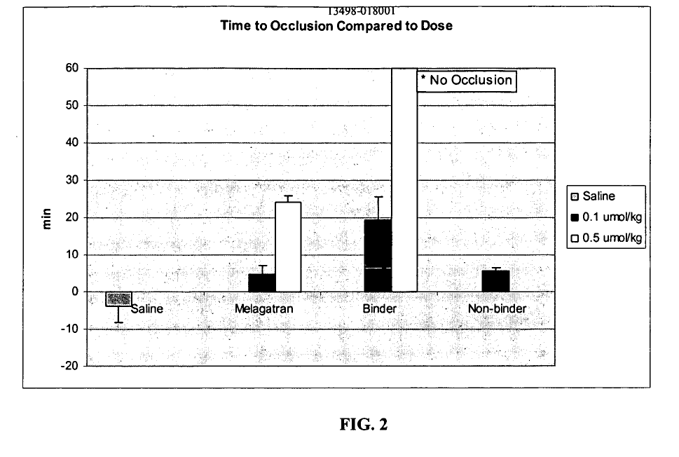

FIG. 2 exemplifies an increased time to occlusion of approximately 4-fold utilizing Hybrid Molecule 3 (a fibrin-targeted thrombin inhibitor containing melagatran as the bioactive moiety) as described in Example 5 (“binder”). The “Non-binder” is the equivalent molecule with the exception that the cysteine at the c-terminal end of the peptide is switched to the d-amino acid form. A comparison of Hybrid Molecule 3's results as compared to an equivalent dose of melagatran alone or the non-binder is shown.

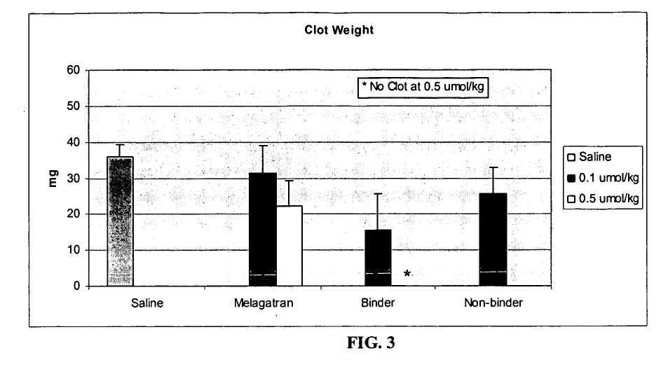

FIG. 3 exemplifies a decrease in clot weight of approximately 2-fold utilizing Hybrid Molecule 3 as compared to an equivalent dose of melagatran or Non-binder control.

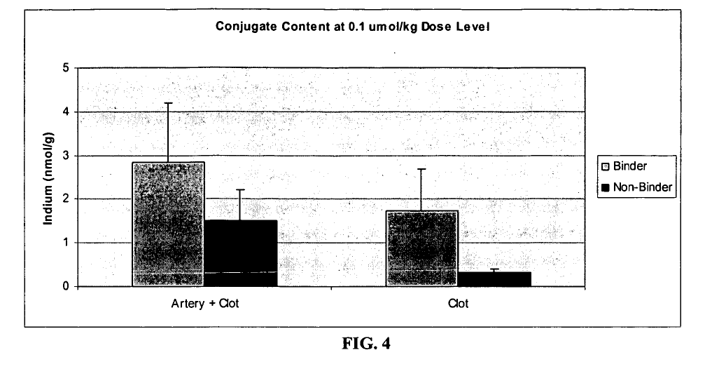

FIG. 4 exemplifies a higher level of Hybrid Molecule 3 compared to an equivalent dose of Non-binder control bound to clot (5-fold) or to clot plus damaged artery (2-fold).

### Example No. 12

In Vivo Efficacy of Fibrin-targeted Inhibitors: A-V Shunt Rabbit Model Rabbits are initially sedated with a subcutaneous injection of Xylazine (5 mg/kg). Approximately 5 min following sedation, the animals are anesthetized with a dose of Ketamine (50 mg/kg) and Acepromazine (2.5 mg/kg) intramuscularly to achieve the appropriate level of anesthesia. Anesthetic depth is assessed using the pain withdraw reflexes and EKG. Pentobarbital anesthesia is given intravenously every 45-60 min at 5-10 mg/kg to maintain proper anesthetic depth.

To form the arterio-venous (A-V) shunt, the left carotid artery and right jugular vein of animals are isolated using a ventral midline approach and cannulated with PE160 tubing. Prior to attaching the tubing to the vessels, a 2 inch length of thrombin-soaked suture thread is placed within the tubing (PE90), which is connected to the PE160 at both ends. After vessel cannulation, ¹²⁵I-fibrinogen (30 μCi/kg) is injected intravenously. Five min post-injection, vessel clamps are released to start arterial-venous shunt. The left femoral vein is isolated and cannulated with PE-20 tubing to administer infusion of test and control compounds. The right femoral artery is isolated and cannulated with EP-20 tubing for blood sampling. The saline, control or test compounds are given as an intravenous bolus injection with or without subsequent continuous infusion either immediately (simultaneous model) or 5 min after (delayed model) A-V shunt release. Blood samples are collected at 1, 3, 5, 12, 20, 30, 45, and 60 min after compound injection. The time to vessel occlusion is monitored using a Doppler flow probe, which is attached to the carotid artery. Upon completion of the procedure, the animals are euthanized with an overdose of intravenous pentobarbital (120 mg/kg). Clot is isolated from PE90 tubing and weighed. Concentration of test compound and fibrin in the clot are determined by γ-countering. Pharmacokinetics of compound are analyzed by using the WinNonLin program.

### Example No. 13

### In Vivo Efficacy of Fibrin-targeted Inhibitors: Venous Thrombosis Model

Rabbits are initially sedated with a subcutaneous injection of Xylazine (5 mg/kg). Approximately 5 min following sedation, the animals are anesthetized with a dose of Ketamine (50 mg/kg) and Acepromazine (2.5 mg/kg) intramuscularly to achieve the appropriate level of anesthesia. Anesthetic depth is assessed using the pain withdraw reflexes and EKG. Anesthesia will be given intravenously every 45-60 minutes at 5-10 mg/kg to maintain proper anesthetic depth.

The femoral artery is isolated and catheterized with polyethylene tubing PE50 for blood collection, to monitor blood pressure and heart rate. The jugular vein is isolated for thrombus placement. For venous thrombus induction 1.9 units 50 μL human thrombin, 45 μL 0.25 mM CaCl₂ and 200 μL whole rabbit blood is prepared in a syringe. The jugular vein is segmented with micro vascular clips, 3 mm between clips. Approximately 100 μL of thrombus mixture is injected between the clips. Clips were removed after 5 min. The saline, control or test compounds are given as an intravenous bolus injection immediately after removal of the clips. Blood sample are collected at 1, 3, 5, 12, 20, 30, 45, and 60 min after compound injection. At the end of experiment, animals are sacrificed with an overdose of pentobarbital (120 mg/kg). Clot is isolated from the vein and weighed. Concentration of test compound and fibrin in the clot are detected by y-countering. Pharmacokinetics of compound are analyzed by using WinNonLin program.

### Example No. 14

### Preparation of Urokinase Tethered to a Fibrin Binding Peptide

Fibrin targeted urokinase (9) is prepared according to the following procedure. A fibrin binding peptide with a Gly-Gly dipeptide linker is prepared according to well established solid phase procedures. The N-terminus of the peptide is blocked with an acetyl group. The C-terminal carboxylic acid is converted to a succinamidal active ester. Direct chemical ligation is achieved by mixing urokinase and the activated peptide in appropriate proportions in an aqueous buffer and gently agitating the solution for 30 minutes.

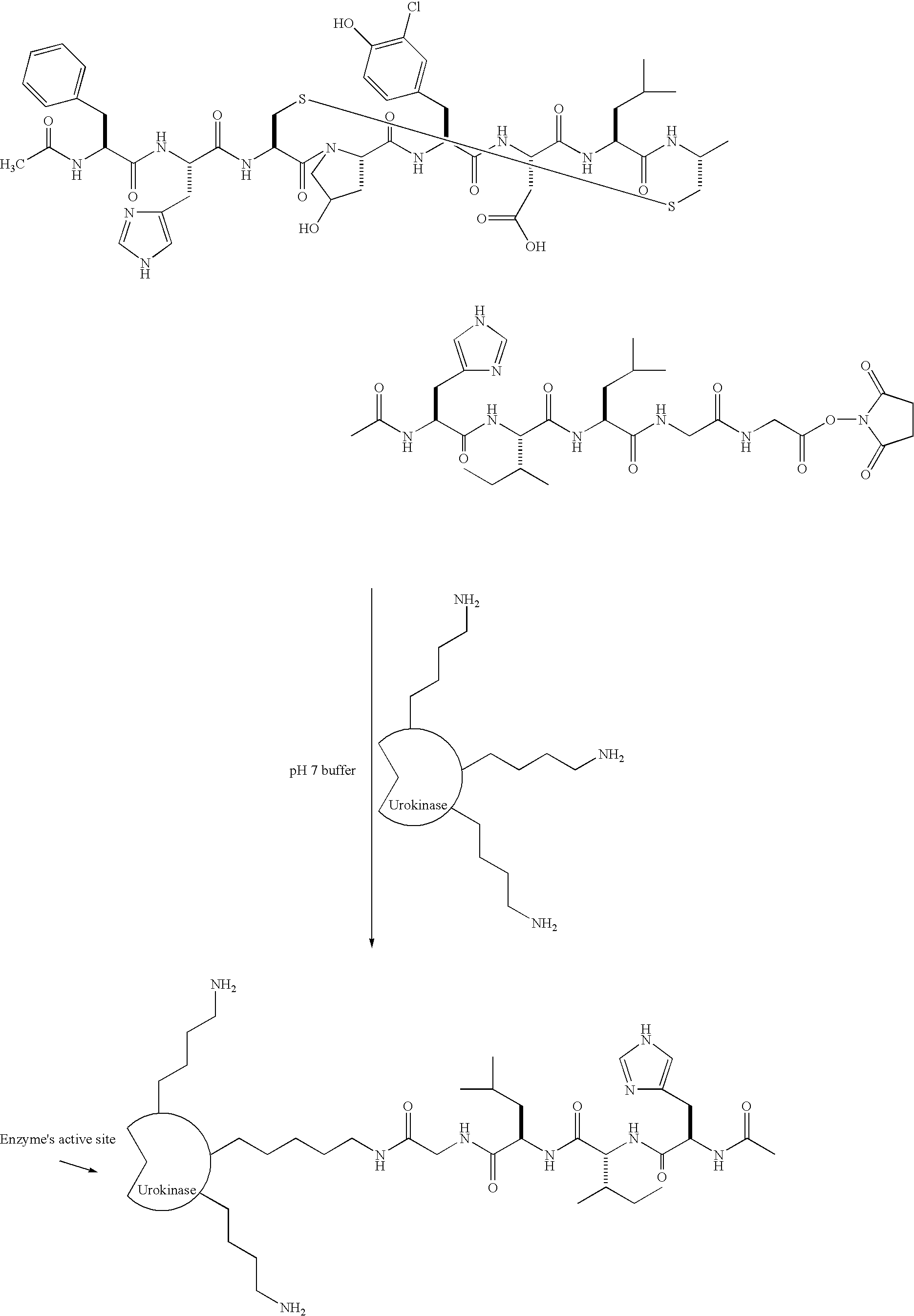

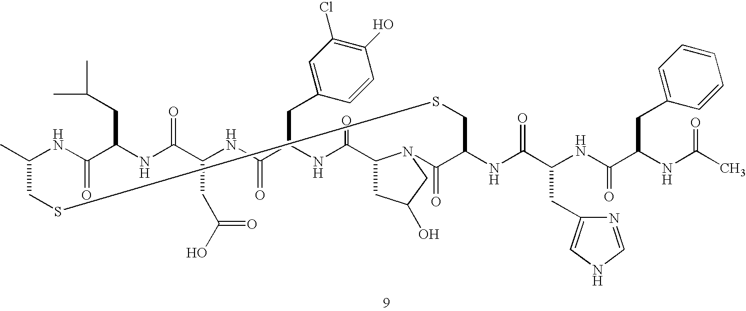

### Example No. 15

### Preparation of Penicillin Tethered to a Fibrin-targeting Peptide

This example shows a preparation of penicillin tethered to a fibrin-targeting peptide using a modification the procedure of Daehne et al. (Daehne, W. et al., J. Med. Chem., 1970, 13, 607-612).

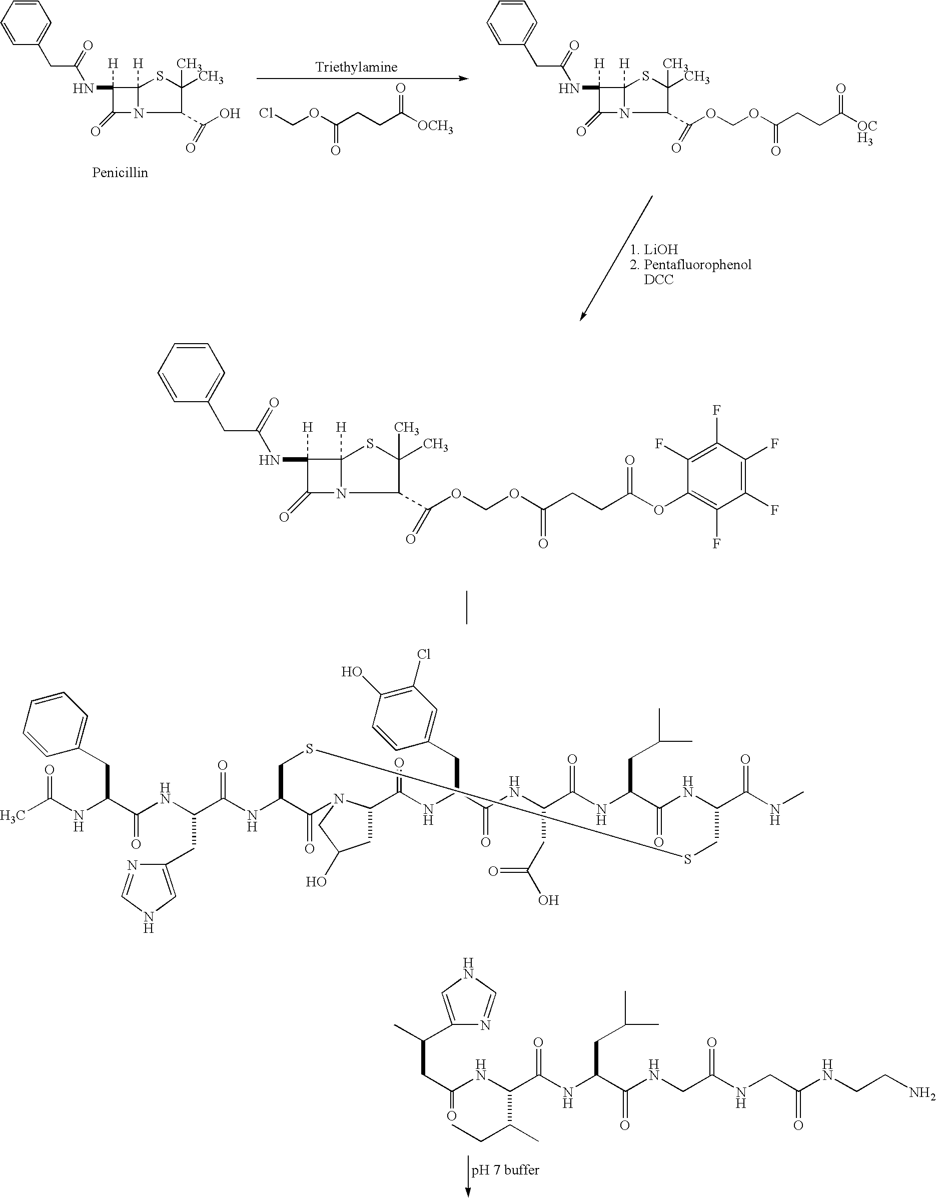

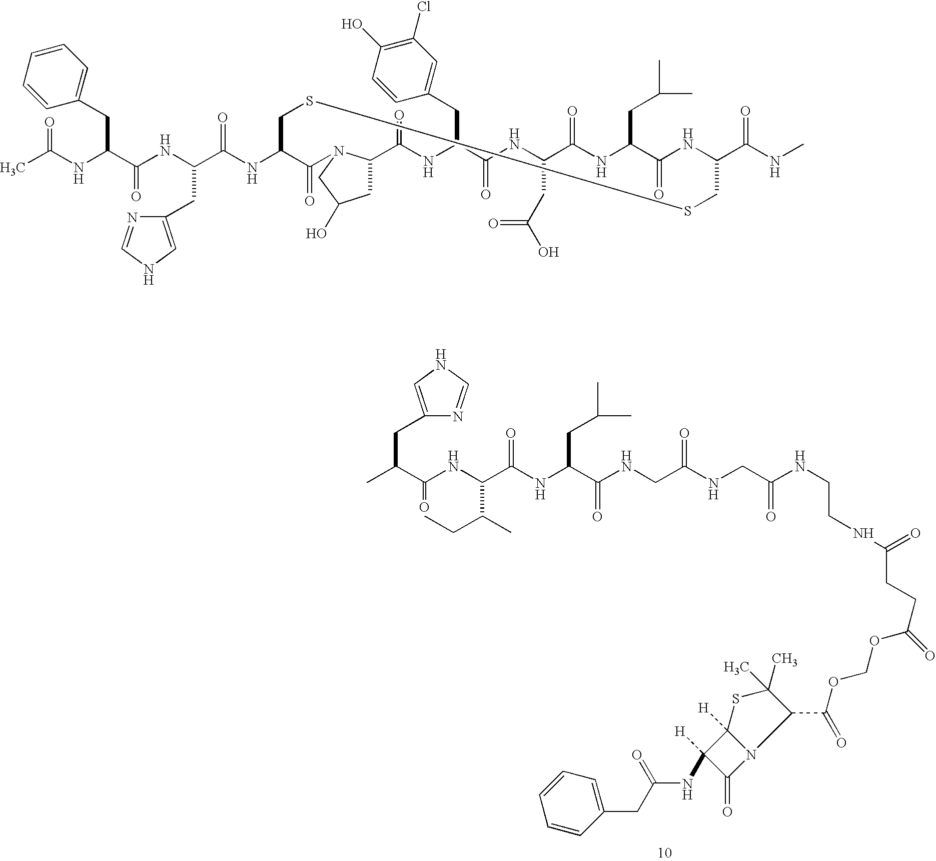

### Example No. 16

### Preparation of Vancomycin Tethered to a Fibrin-targeting Peptide

This example shows a preparation of vancomycin tethered to a fibrin-targeting peptide (11).

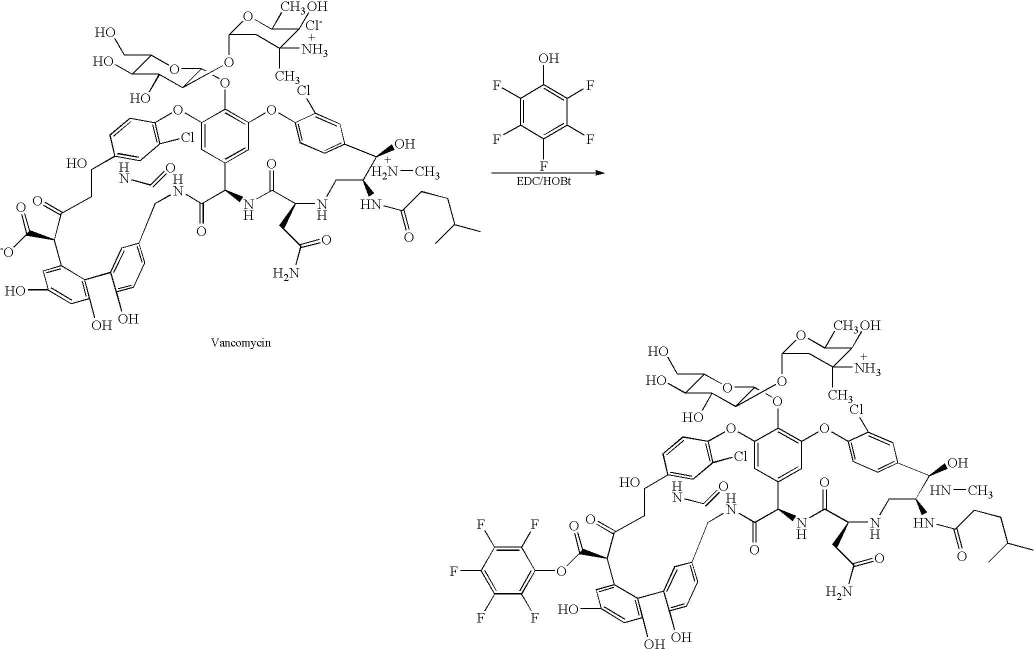

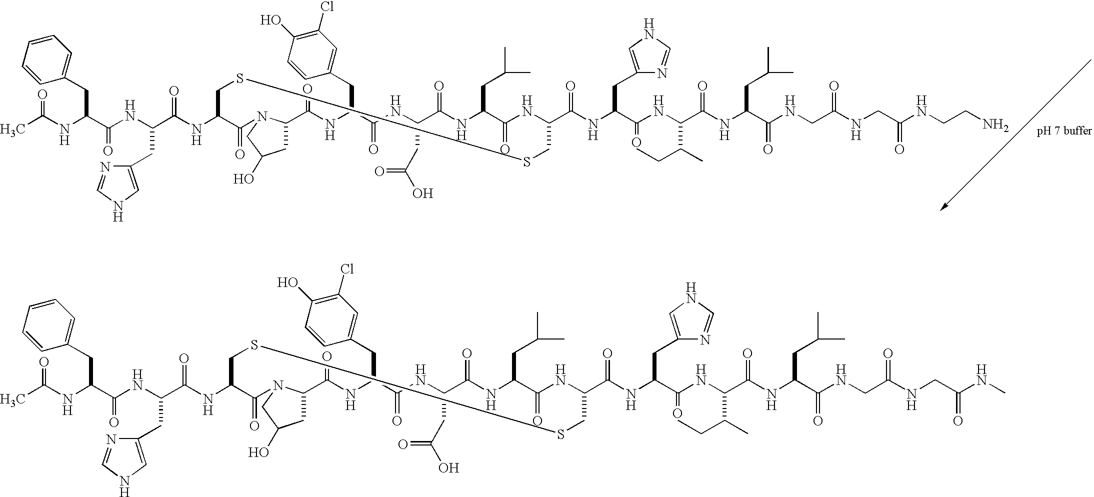

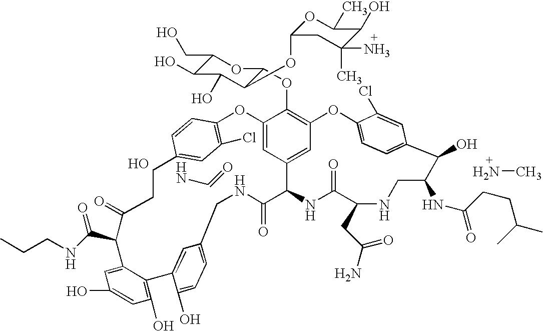

### Example No. 17

### Preparation of Paclitaxel Conjugated to a Fibrin Binding Peptide (Compound 12)

A conjugate of paclitaxel and a fibrin-targeting peptide (12) was prepared according to modifications of a literature procedure (Chun-Ming Huang, et al., Chem. Biol. 2000, 7, 453-461).

Paclitaxel (100.0 mg) and succinic anhydride (141.0 mg) were dissolved in pyridine (3.0 mL). The mixture was stirred at room temperature for 3.0 hours. The pyridine was removed under vacuum. Ice-cold water (6.0 mL) was added. The mixture was stirred for 20 minutes. The solid precipitate was collected by filtration, and was dissolved in acetone (10 mL). Water was added to this solution until the solution became slightly cloudy. At this point, the container was covered, and crystallization was allowed to proceed. The resultant white crystals were collected by filtration, and dried in open air. 98.1 mg of the product was obtained.

The paclitaxel succinate (98.1 mg) was dissolved in methylene chloride (2.5 mL). A solution of pentafluorophenol (28.4 mg) in methylene chloride (2.5 mL) was added. The mixture was cooled to 0° C. EDCI (29.6 mg) was added. The resultant mixture was stirred at room temperature for 3.0 hours. This mixture was washed with dilute brine (3×10 mL), dried over magnesium sulfate, and concentrated to give a solid residue.

The solid residue thus obtained was dissolved in DMF (5 mL). The peptide (100.0 mg) was added. The pH of the solution was adjusted to 7-8 with triethylamine. The reaction mixture was stirred at room temperature overnight. The DMF was removed under vacuum. The crude residue was purified by preparative HPLC to yield 12.0 mg of the desired conjugate as a white powder.

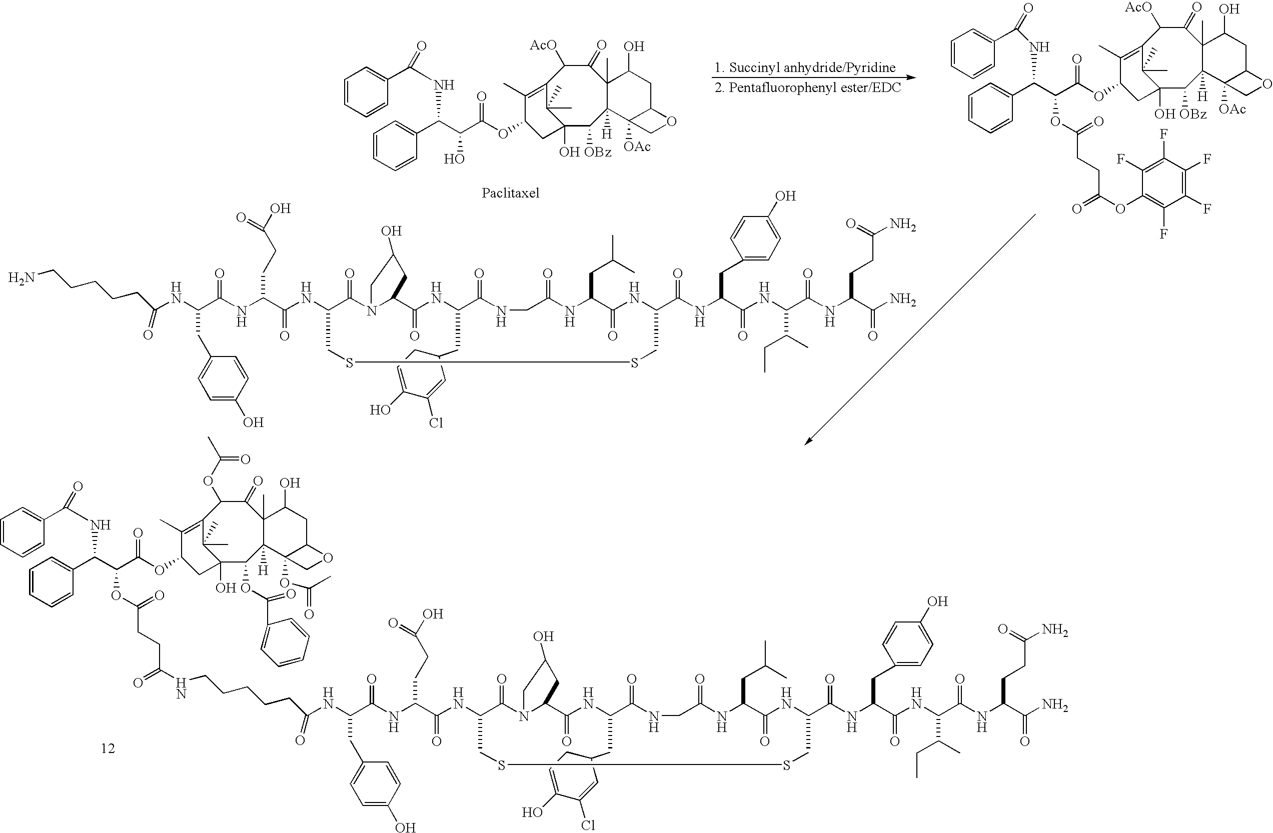

### Example No. 18

### Preparation of Doxorubicin Conjugated to a Fibrin Binding Peptide

Doxorubicin can be conjugated to a fibrin binding peptide to generate conjugate (13) using the pentafluorophenyl ester activation method. The two moieties in this specific example can be connected through a peptide linker. The linker peptide can be cleaved in vivo by MMP-2, a proteolytic enzyme that is up-regulated at tumor sites.

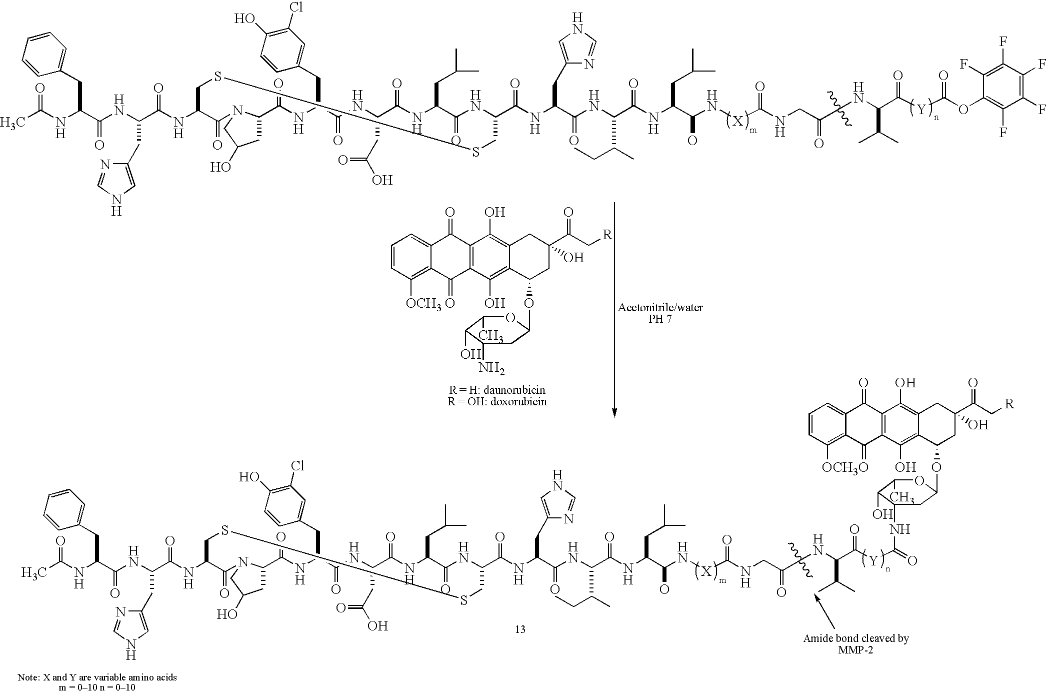

### Example No. 19

### Preparation of a Fibrin Binding Peptide-DTPA radionuclide

A conjugate (14) of a fibrin binding peptide and DPTA-radionuclide chelate complex can be prepared according to the following scheme.

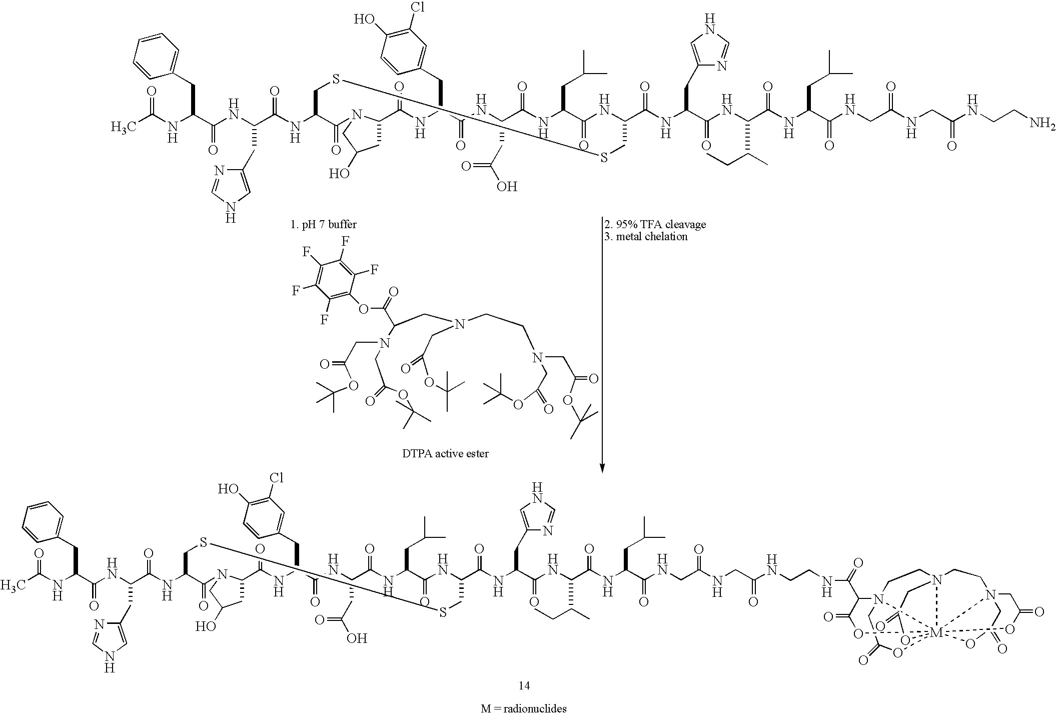

### Example No. 20

### Preparation of a Fibrin Binding Peptide-DTPA radionuclide

A conjugate (15) of a fibrin binding peptide and DPTA-radionuclide chelate complex can be prepared according to the following scheme.

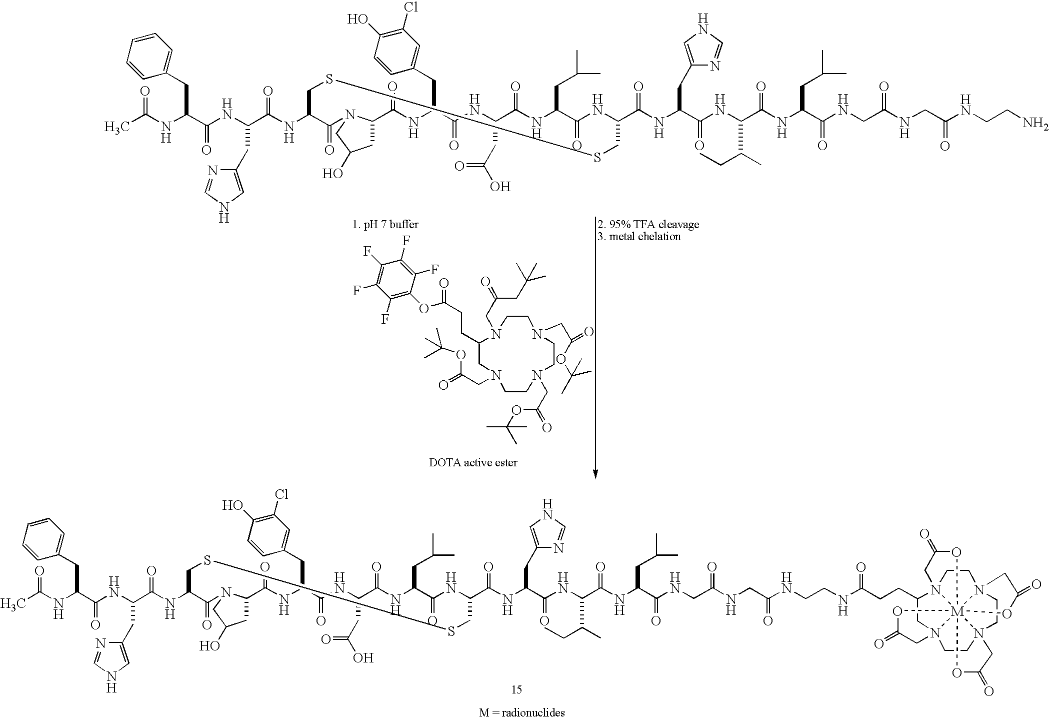

### Example No. 21

### Preparation of Lipids for Fibrin-targeting Liposomes

A lipid can be conjugated to a fibrin-targeting peptide according to the following scheme to yield a conjugate (16) for use in fibrin-targeting liposomes.

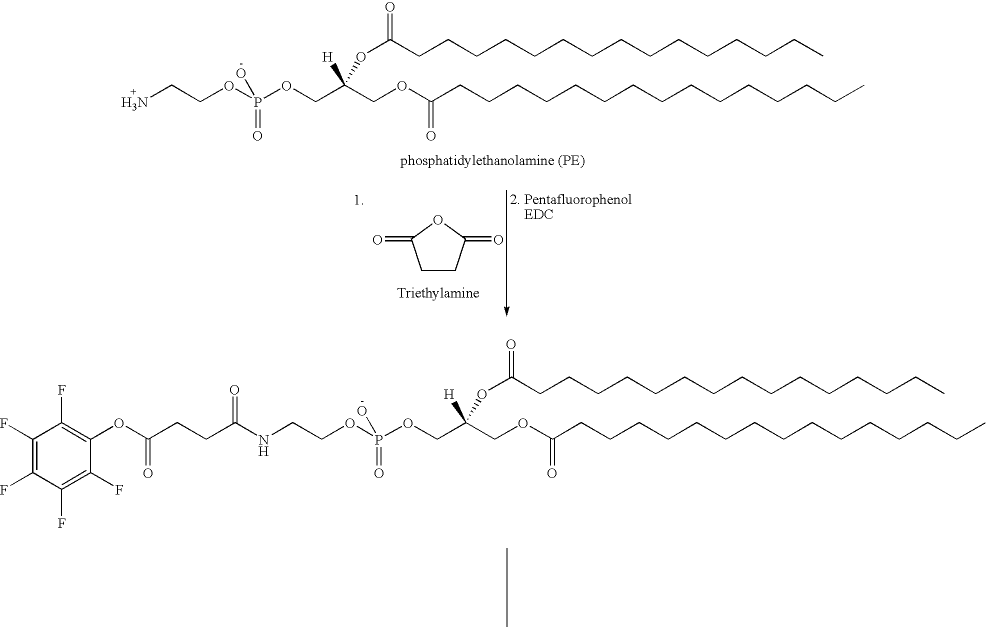

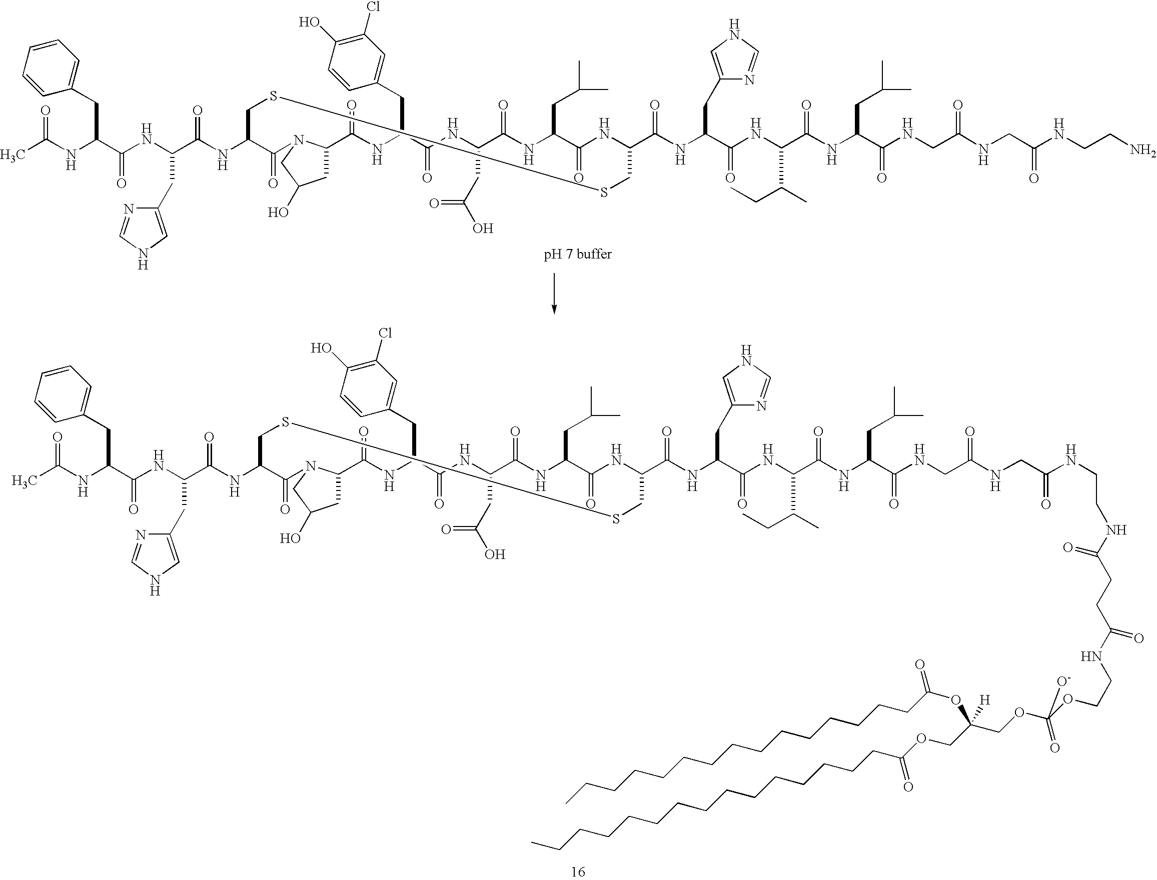

### Example No. 22

### In Vivo Tumor Uptake of a Fibrin Binding Peptide

A high affinity fibrin binding peptide (Structure 8 in Scheme 1) was labeled with ¹⁵³Gd. Selective tumor uptake of this fibrin binding peptide was demonstrated using the mouse Lewis lung metastatic model. In this model mice were intravenously injected with Lewis tumor cells. Sixteen days later, ¹⁵³Gd-labeled agent (2.5 μmol/kg,i.v.) was administered to both tumor bearing and nontumor bearing (control) mice. Forty-five minutes post-injection, the animals were euthanized and lungs excised. The uptake of ¹⁵³Gd-labeled agent into the tumor was determined using gamma scintillation counting. Differential accumulation of fibrin in the tumor was observed.

### Example No. 23

### In Vitro and In Vivo Infective Endocarditis Disease Models

In vitro Simulated Endocardial Vegetations

This model is a high throughput in vitro screen where the ability of an agent to inhibit bacterial growth can be determined. Bacteria stocks are grown on agar. When a growth rate of 10¹⁰ CFU/ml is achieved, the colonies are harvested. Simulated vegetations are prepared by mixing the bacteria with plasma. A silk thread is placed in the media and the media activated using thrombin. The thread is removed and suspended in a beaker that is prefilled with buffer and various concentrations of the test article stirred at a constant rate and temperature (37° C.). At the appropriate time the thread is removed and the growth homogenized and plated. 24 hrs later colony counts are performed and time-kill curves plotted.

In vivo Infected and Noninfected Fibrin Thrombus Model

This model is a high throughput screen where the pharmacokinetics of the agent and preliminary in vivo efficacy are determined. Rabbits are anesthetized and fibrin clots containing 10⁸ CFU/g of bacteria are placed in a subcutaneous pocket in the animals flank. The test article is administered and bacterial viability assessed at various times post-treatment by analysis of the bacterial content of each clot. The rate of bacteria kill is determined by calculating the difference in bacterial counts and that of the initial inoculum (change log 10 CFU/g). These results are compared to the bacteria negative clots.

Rabbit Endocarditis Model

This is a secondary disease-state model that can be used for determination of efficacy of an agent in an intended clinical application. Left-sided endocarditis can be established by using an intravenous bacterial inoculum of 10⁶ CFU. In this model, a catheter is placed across the aortic valve and remains in place for the duration of the study. Sixteen hours after bacterial challenge, 1 mL of blood is withdrawn from all animals for culture. Serial dilution and plating techniques can be used to determine the number of CFU per milliliter of blood. Inclusion in the study requires that the blood culture be positive and that the catheter be positioned properly across the aortic valve at the time of autopsy. Rabbits are randomized to receive treatment or control and euthanized in matched pairs. Plasma samples for the measurement of blood levels are obtained throughout the study. Blood cultures are obtained prior to the first dose on day 3 and following 4 days of therapy. Following 4 days of therapy all animals are euthanized, and terminal blood cultures and plasma samples for the measurement of antibiotic content are obtained, followed by the removal of vegetations and 500-mg (mean weight) sections of left kidney and spleen for culture. These specimens are weighed, suspended in 0.9% NaCl (final volume, 1 ml), and homogenized. Quantitative bacterial counts, determined by serial dilution and plating techniques, are expressed as the log 10 of the number of CFU per gram.

### Example No. 24

### Efficacy of Fibrin Targeted Urokinase Thrombosis Plasma Activator

Fibrin targeted urokinase (Compound 9 in Example 14) is purified by HPLC. Compound 9 in Example 14 binds fibrin selectively versus fibrinogen. The rabbit jugular vein model of Collen et al. (Collen, D., et al., J. Clin. Invest. 1983, 71, 368-376) can be used for thrombolysis assays. 2 mg/kg of compound 9 is administered by infusion of a bolus (consisting of 20% of the total dose) over 1 min, along with a heparin bolus (300 units/kg) over 1 min. The remainder of the compound 9 dose is continuously infused over the next 60 min, and heparin (60 units/kg/hr) continuously infused over the next 180 min. At 3 hours, the animals are sacrificed, and clots are analyzed. Compound 9 is more potent in clot lysis than single chain urokinase plasminogen activator (scuPA) alone. At 3 hr with 2 mg/kg of compound 9, there is less consumption of fibrinogen and α-antiplasmin, relative to equivalent doses of scuPA alone, demonstrating that compound 9 is more fibrin specific than scuPA alone.

### Example 25

### Efficacy of Fibrin Targeted Vancomycin

Fibrin targeted vancomycin (Compound 11 in Example 16) is purified by HPLC. Compound 11 binds fibrin selectively versus fibrinogen.

A rabbit endocarditis model is created using an intravenous bacterial inoculum of 106 CFU into the myocardium, as described in Example 22. Treatment of the animal with 30 mg/kg of Compound 11 given intravenously in two equally divided doses over 24 hours results in a lower bacterial count in the lesion after 4 days, relative to an animal receiving the equivalent doses of vancomycin alone.

### Example 26

### Efficacy of Fibrin Targeted Doxorubicin

Fibrin targeted doxorubicin (Compound 13 in Example 18) is purified by HPLC. Compound 13 binds fibrin selectively versus fibrinogen. Compound 13 (26.6 pmol) is incubated with 6 U (30 mg) of MMP-2 activated with 2.5 mM of p-aminophenyl mercuric acid—APMA) for 24 hours. Samples are analyzed by LC-MS, and enzymatic cleavage is confirmed. As a control, the conjugate with a peptide linker that was not a MMP-2 substrate shows no traces of cleavage products. 2×10⁶ HT1080 fibrosarcoma cells are injected subcutaneously in the mammary fat pad of athymic nude mice (nu/nu, 10 weeks old). Twenty-four hours later, 2.8 mmol/kg of fibrin targeted doxorubicin is injected i.v. once a week for 5 consecutive weeks. At the end of 5½ weeks, the animals are sacrificed, and the tumors are excised. The mice treated with fibrin targeted dox exhibit smaller tumors as compared to the ones treated with equivalent doses of dox alone.

### Example 27

### Effect of Compound 12 on Tumor, Endothelial, and Smooth Muscle Cells

The effect of Compound 12 (see Example 17 above) on the viability of two tumor cell lines (A549 lung and ZR-75-1 breast) as well as human umbilical vein endothelial (HUVEC) cells and vascular smooth muscle (SMC) cells was investigated. The four different cell lines were incubated with Compound 12 or vehicle alone (e.g., 0 nM Compound 12) for 48 hours at 3° C. All experiments were performed in duplicate with N=3 for A549, ZR-75-1, SMC lines and N=2 for the HUVEC line. All values are mean ± SEM.

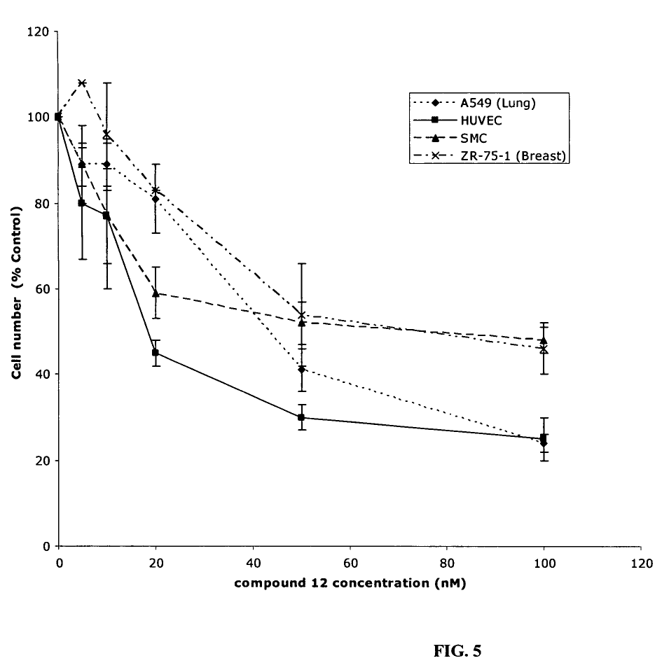

FIG. 5 shows the percent of viable cells relative to those incubated with the vehicle alone (O nM Compound 12). As can be seen, Compound 12 in a dose-responsive manner caused a significant decrease in the number of cancer cells in both the lung cancer and breast cancer lines, as well as a significant decrease in the number of smooth muscle and endothelial cells. Accordingly, certain compounds of the invention, including Compound 12, can be used to treat cancer as well as to inhibit vascular smooth muscle cell (VMSC) proliferation and migration. Such compound can be used to inhibit or prevent stenosis or re-stenosis, e.g., after percutaneous coronary angioplasty, stent placement, or other percutaneous interventions.

### Example 28

### Design of Fibrin-targeted Factor Xa Inhibitors

The Factor Xa inhibitors below can be conjugated to a fibrin-targeting moiety, such as a peptide described previously, to result in a fibrin-targeted Factor Xa inhibitor. The arrows denote possible sites of conjugation to a fibrin targeting moiety.

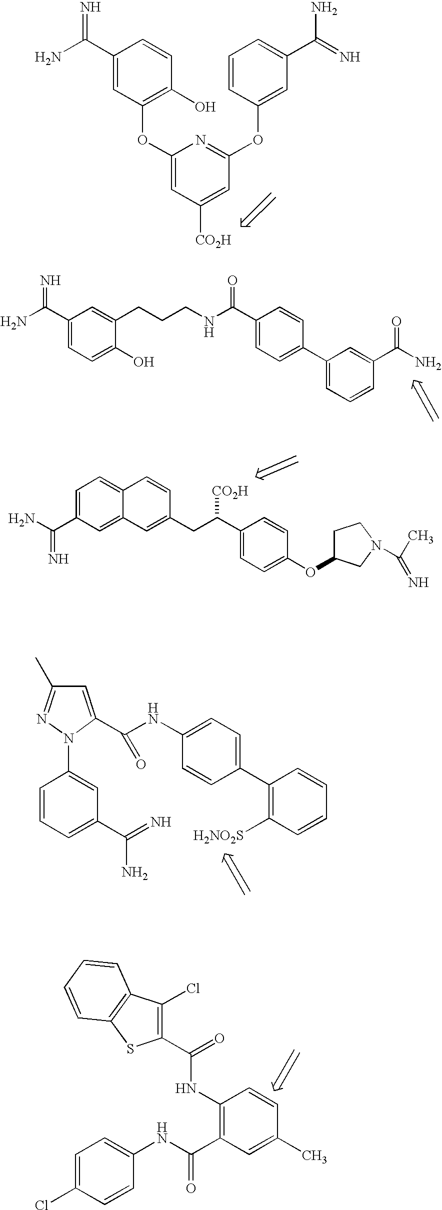

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A hybrid molecule of the general formula:
[D]ᵢ-[L]ⱼ-[F]ₖ, wherein [D] comprises a bioactive moiety for treating thromboembolism, infection, or cancer, [L] comprises a linker moiety, and [F] comprises a fibrin-targeting moiety, wherein i and k are integers between 1 and 20, and j can be an integer from 0 to 20.

2. The hybrid molecule of claim 1, wherein the fibrin-targeting moiety is a peptide.

3. The hybrid molecule of claim 1, wherein the fibrin-targeting moiety is a peptidomimetic.

4. The hybrid molecule of claim 1, wherein the fibrin-targeting moiety is a small molecule.

5. The hybrid molecule of claim 1, wherein the fibrin-targeting moiety is a peptide having 2 to about 20 amino acids.

6. The hybrid molecule of claim 2, wherein the peptide comprises the amino acid sequence: P*Y*-X₁*-L*, wherein:
P* is a proline or a non-natural derivative thereof;
Y* is a tyrosine or a non-natural derivative thereof;
X₁* is G or D or a non-natural derivative of G or D;
L* is a leucine or a non-natural derivative thereof.

7. The hybrid molecule of claim 6, wherein X₁* is G or D and wherein L* is leucine.

8. The hybrid molecule of claim 6, wherein P* is proline or 4-hydroxyproline, and wherein Y* is tyrosine or a non-natural derivative of tyrosine substituted at the 3 position with a moiety selected from the group consisting of F, Cl, Br, I, and NO₂.

9. The hybrid molecule of claim 2, wherein the peptide comprises the amino acid sequence:
X₁-X₂-C-P*-Y*-X₃-L-C-X₄-X₅-X₆, wherein:
P* is a proline or a non-natural derivative thereof;
Y* is a tyrosine or a non-natural derivative thereof;
X₁ is selected from the group consisting of W, Y, F, S, Bip, Hx, Dpr, Cy, Gu, Ad, Hfe, 3-Pal, 4-Pal, DopaMe₂, nTyr, dW, dF, F(3/4*), and Y(3*), wherein F(3/4*) is a phenylalanine substituted at either the 3 or the 4 position with a moiety selected from the group consisting of CH₃, CF₃, NH₂, CH₂NH₂, CN, F, Cl, Br, I, Et, and OMe, and wherein Y(3*) is a tyrosine substituted at the 3 position with a moiety selected from the group consisting of F, Cl, Br, I, and NO₂;
X₂ is selected from the group consisting of E, H, dE, S, H(Bzl), 2-Pal, Dpr, and Th;
X₃ is selected from the group consisting of G and D;
X₄ is selected from the group consisting of H, F, Y, and W;
X₅ is selected from the group consisting of I, L, V, N, Bpa, Bal, Hfe, Nle, Tle, Nval, Phg, Cha, Taz, Fua, Th, 4-Pal, and F(3/4*), wherein F(3/4*) is a phenylalanine substituted at either the 3 or the 4 position with a moiety selected from the group consisting of CF₃, Et, iPr, and OMe;
X₆ is selected from the group consisting of N, Q, I, L, and V, or X₆ is not present.

10. The hybrid molecule of claim 9, wherein X₄ is H.

11. The hybrid molecule of claim 9, wherein P* is proline or 4-hydroxyproline, and Y* is tyrosine or a non-natural derivative of tyrosine substituted at the 3 position with a moiety selected from the group consisting of F, Cl, Br, I, and NO₂.

12. The hybrid molecule of claim 2, wherein the peptide comprises the amino acid sequence: C-P*-Y*-X₁-L-C, wherein:
X₁ is G or D,
P* is proline or its non-natural derivative 4-hydroxyproline;
Y* is tyrosine or a non-natural derivative of tyrosine substituted at the 3 position with a moiety selected from the group consisting of F, Cl, Br, I, and NO₂.

13. The hybrid molecule of claim 2, wherein the peptide comprises the amino acid sequence: C-D-Y-Y-G-T-C-X₁₀, wherein:
X₁₀ is selected from the group consisting of n(decyl)G, n(4-PhBu)G, MeL, Bpa, Bip, Me-Bip, F(4*), F(3-Me), F(3,4-difluoro), Amh, Hfe, Y(3,5-di-iodo), Pff, 1Nal, d1Nal, and MeL, wherein F(4*) is a phenylalanine substituted at the 4 position with a moiety selected from the group consisting of Et, CF₃, I, and iPr.

14. The hybrid molecule of claim 2, the peptide comprising the amino acid sequence: C-D-Y-Y-G-T-C-X₁₀-X₁₁, wherein:
X₁₀ is selected from the group consisting of n(decyl)G, n(4-PhBu)G, MeL, Bpa, Bip, Me-Bip, F(4*), F(3-Me), F(3,4-difluoro), Amh, Hfe, Y(3 5-di-iodo), Pff, 1Nal, d1Nal, and MeL, wherein F(4*) is a phenylalanine substituted at the 4 position with a moiety selected from the group consisting of Et, CF₃, I, and iPr; and
X₁₁, is selected from the group consisting of D, dD, βD, Inp, Nip, Me-D, dC, Cop, and Cmp.

15. The hybrid molecule of claim 2, wherein the peptide is selected from the group consisting of:

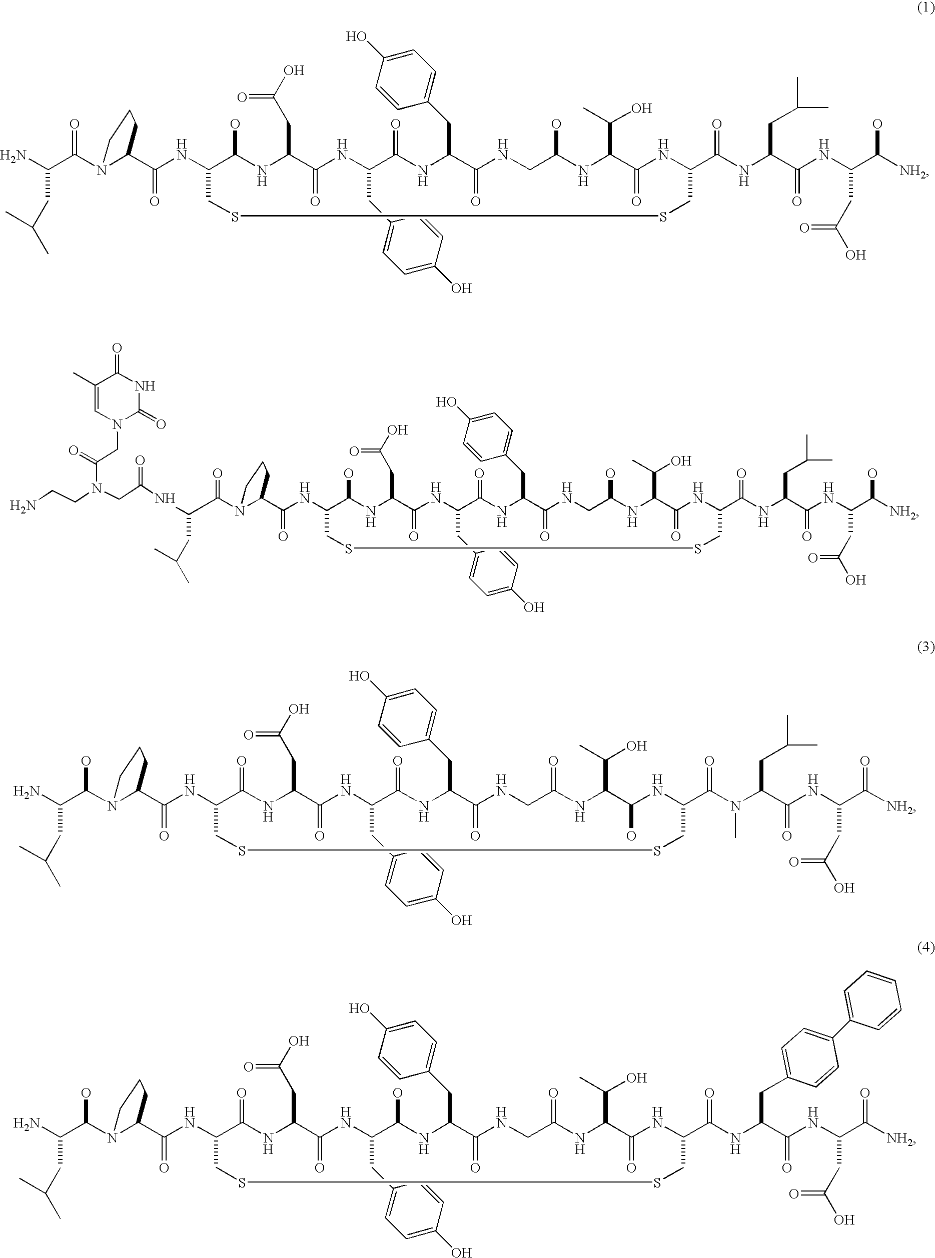

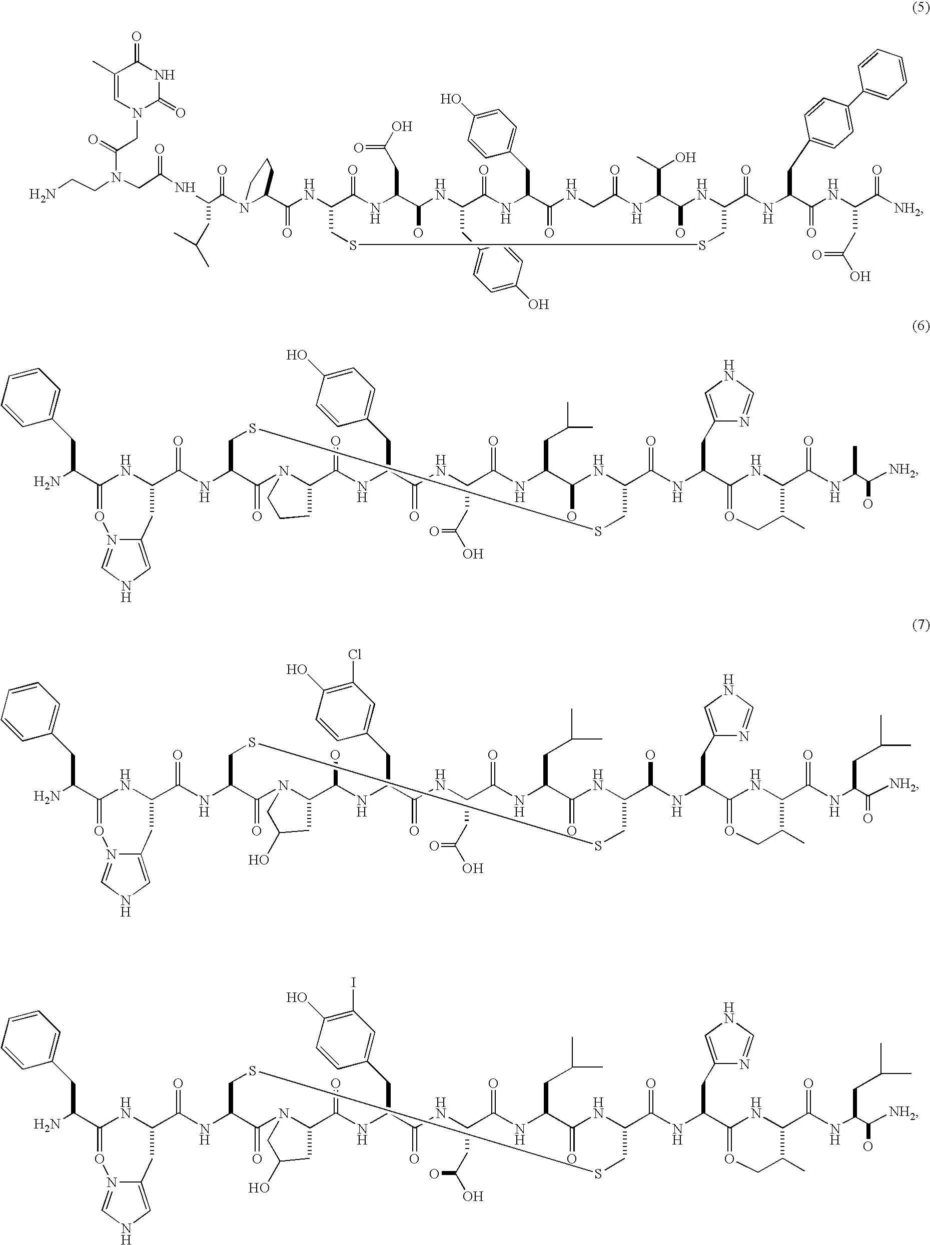

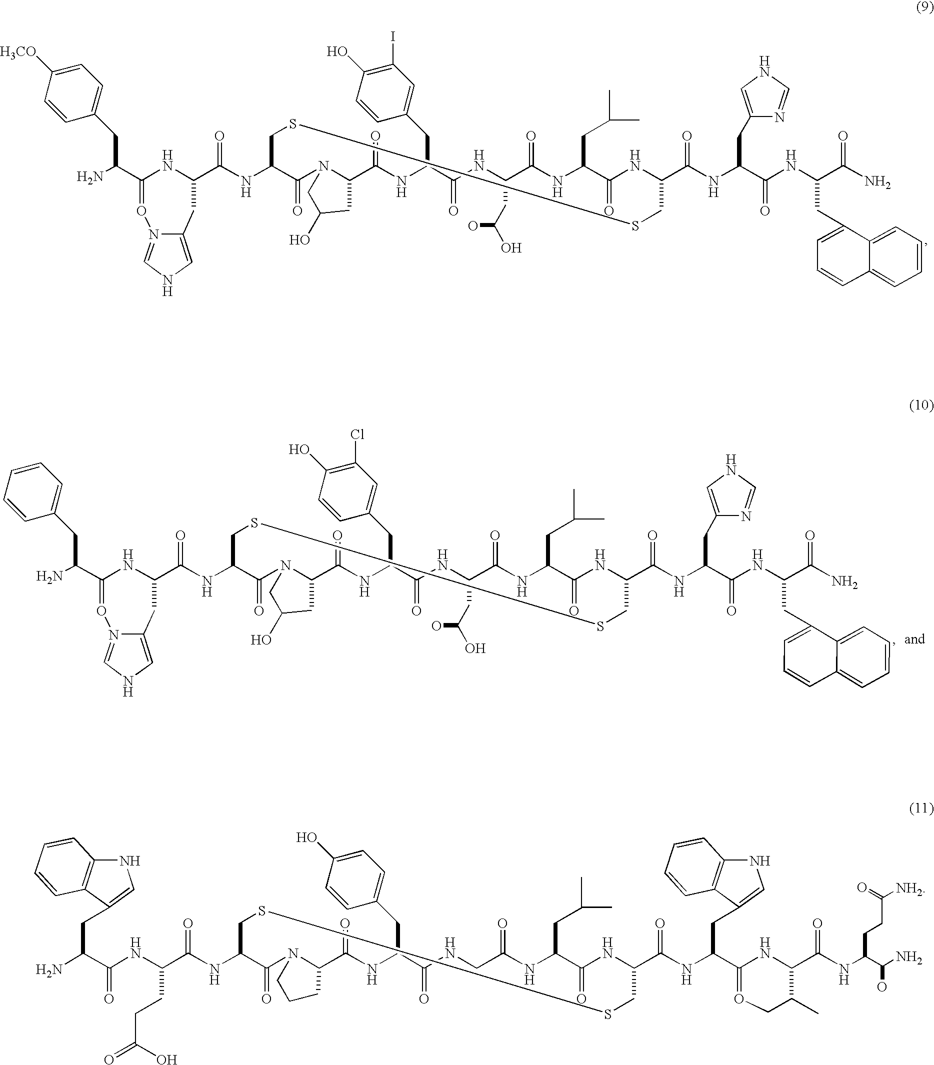

16. The hybrid molecule of claim 1, wherein the bioactive moiety comprises an antithrombotic agent.

17. The hybrid molecule of claim 16, wherein the antithrombotic agent is a thrombolytic, fibrinolytic, antiplatelet, anticoagulant, hyaluronic acid, or dextran.

18. The hybrid molecule of claim 17, wherein the antithrombotic agent is a fibrinolytic.

19. The hybrid molecule of claim 18, wherein the fibrinolytic is copperhead snake fibrolase.

20. The hybrid molecule of claim 17, wherein the antithrombotic agent is a thrombolytic.

21. The hybrid molecule of claim 20, wherein the thrombolytic is selected from the group consisting of a tissue plasminogen activator, urokinase, plasminogen activators (e.g., vampire bat plasminogen activator), streptokinase, staphylokinase, prourokinase, anisolated streptokinase plasminogen activator complex (ASPAC), and animal salivary gland plasminogen activator.

22. The hybrid molecule of claim 17, wherein the antithrombotic agent is an anticoagulant.

23. The hybrid molecule of claim 22, wherein the anticoagulant is selected from the group consisting of a thrombin inhibitor, Factor Xa inhibitor, tissue factor inhibitor, Factor VIIa inhibitor, Factor IXa inhibitor, Factor Va inhibitor, Factor XIa inhibitor, Factor XIIa inhibitor, TAFIα inhibitor, α2-antiplasmin inhibitor, PAI-1 inhibitor, PAI-2 inhibitor, PAI-3 inhibitor, prothrombinase inhibitor, tick anticoagulation peptide, protein C, warfarin, heparin, lepirudin, aspirin, ticlopidine, clopidogrel, tirofiban, and eptifibatide.

24. The hybrid molecule of claim 22, wherein the anticoagulant is selected from the group consisting of:

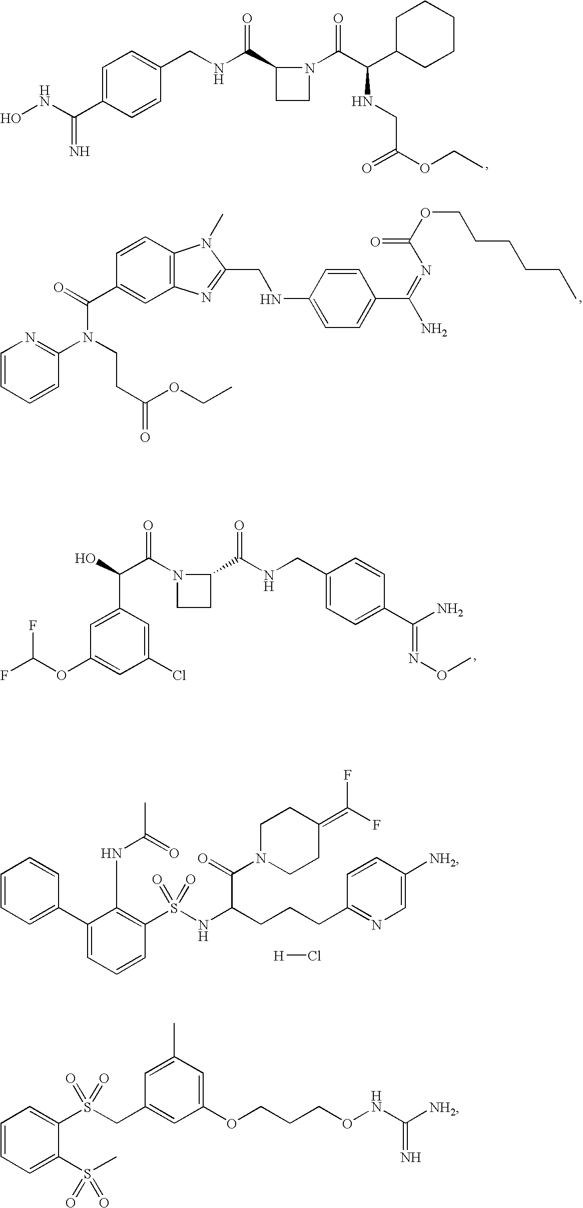

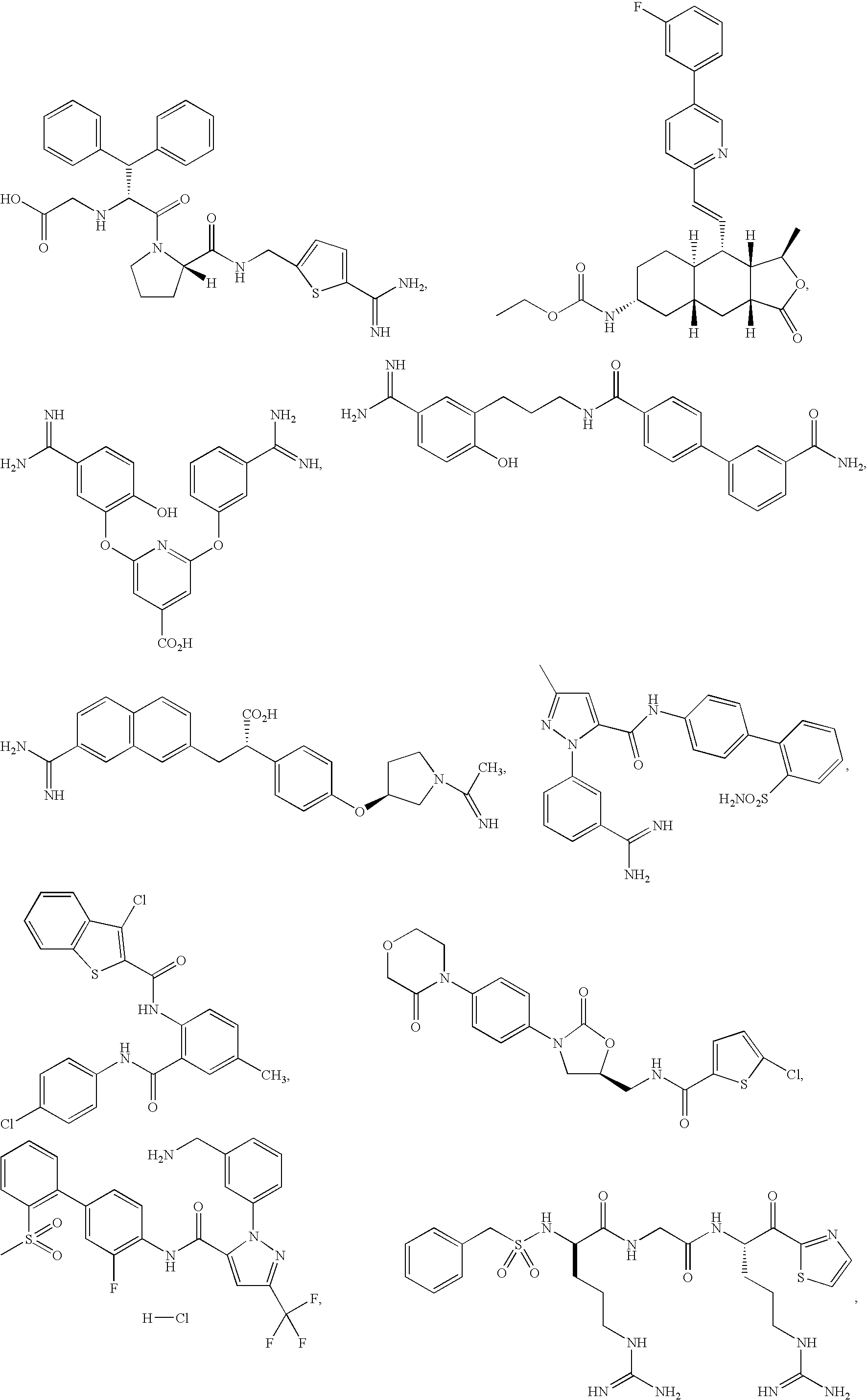

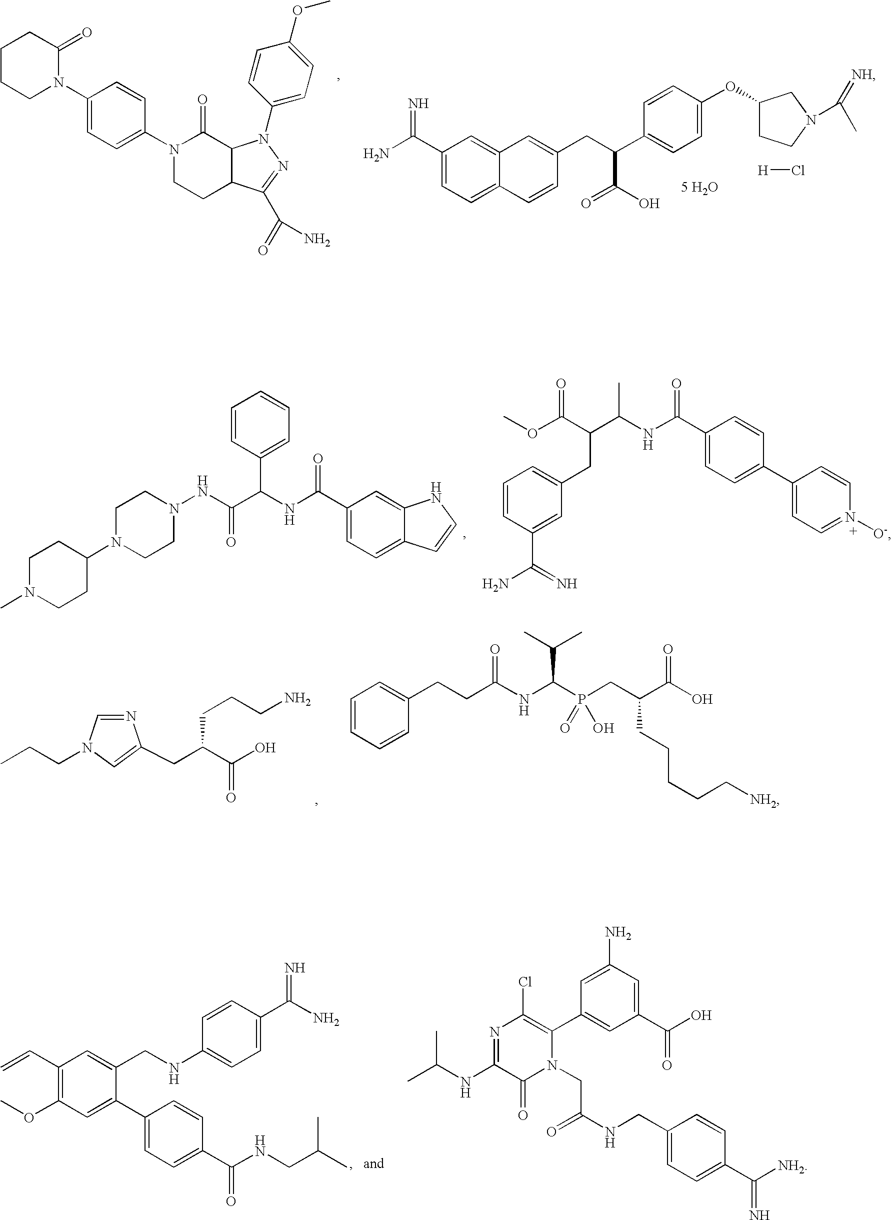

25. The hybrid molecule of claim 17, wherein the anti thrombotic is an antiplatelet.

26. The hybrid molecule of claim 25, wherein the antiplatelet is selected from the group consisting of GPIIb/IIIa receptor inhibitor, ADP receptor inhibitor, thrombin receptor inhibitor, CD40 inhibitor, CD40L inhibitor, Gas6 inhibitor, Gas6 receptor ax1 inhibitor, Gas 6 receptor inhibitor Sky, Gas6 receptor Mer inhibitor, P-selectin inhibitor, P-selectin receptor PSGL-1 inhibitor, thromboxane inhibitor, synthetase inhibitor, fibrinogen receptor antagonist, prostacyclin mimetic, phosphodiesterase inhibitor, RANTES inhibitor, phosphoinositide-3-kinase (PI(3)K) isoform β inhibitor, phosphoinositide-3-kinase (PI(3)K) isoform γ inhibitor,.eptifibatide, tirofiban, ticlopidine, and clopidogrel.

27. The hybrid molecule of claim 25, wherein the antiplatelet is selected from the group consisting of:

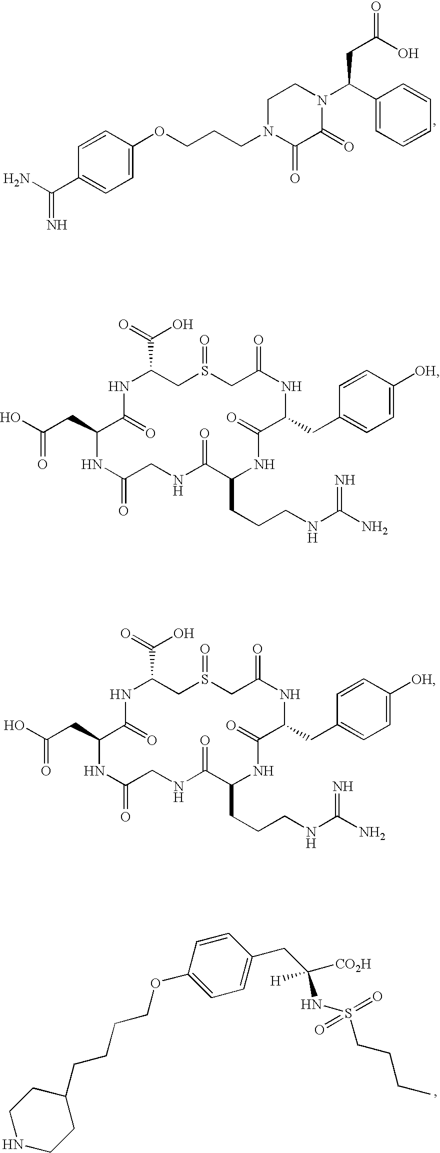

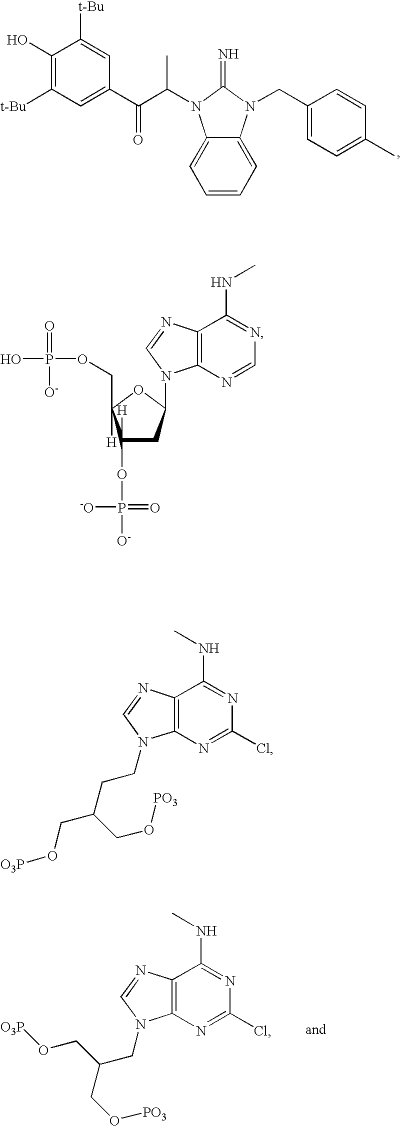

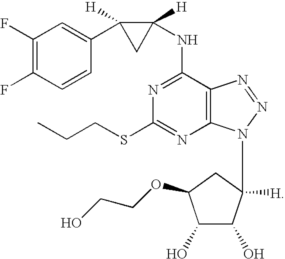

28. The hybrid molecule of claim 1, wherein the bioactive moiety comprises an antibiotic.

29. The hybrid molecule of claim 28, wherein the antibiotic is a β-lactam antibiotic, sulfonamide, quinolone, trimethoprim, aminoglycoside, tetracycline, macrolide, probenecid, chloramphenicol, or glycopeptide.

30. The hybrid molecule of claim 29, wherein the β-lactam is penicillin, cephalosporin, or carbapenem.

31. The hybrid molecule of claim 30, wherein the penicillin is penicillin, ampicillin, nafcillin, oxacillin, or methicillin.

32. The hybrid molecule of claim 30, wherein the cephalosporin is cepahlothin, cefamandole, ceftaxidime, deacetylcephalothin, cephaloridine, cefixime, or latamoxef.

33. The hybrid molecule of claim 29, wherein the macrolide is erythromycin, clarithromycin, or azithromycin.

34. The hybrid molecule of claim 29, wherein the glycopeptides is vancomycin.

35. The hybrid molecule of claim 29, wherein the aminoglycoside is kanamycin or streptomycin.

36. The hybrid molecule of claim 1, wherein the bioactive moiety comprises an antitumor agent.

37. The hybrid molecule of claim 36, wherein the antitumor agent comprises a cytotoxin.

38. The hybrid molecule of claim 37, wherein the cytotoxin is selected from cytotoxic an alkylating agent, antimetabolite, natural product, antibiotic, platinum coordination complex, or antagonist.

39. The hybrid molecule of claim 37, wherein the cytotoxin is selected from the group consisting of: 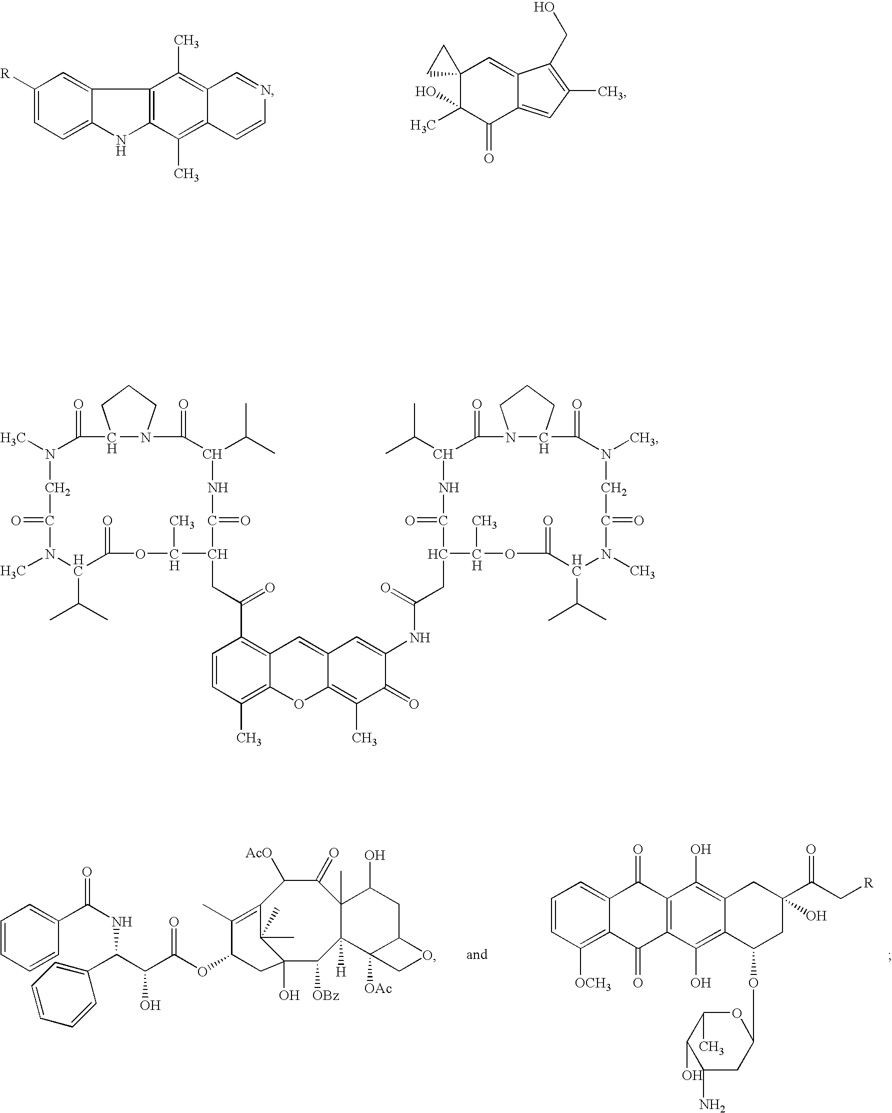
wherein R is H or OH.

40. The hybrid molecule of claim 1, wherein the bioactive moiety comprises a radiopharmaceutical.

41. The hybrid molecule of claim 40, wherein the radiopharmaceutical comprises a radionuclide.

42. The hybrid molecule of claim 41, wherein the radionuclide is selected from Iodine-131, Yttrium-90, Lutetium-177, Copper-67, Rhenium-186, Rhenium-188, Bismuth-212, Bismuth-213, or Astatine-211.

43. The hybrid molecule of claim 41, wherein the radiopharmaceutical comprises a chelating group.

44. The hybrid molecule of claim 1, wherein the linker is cleavable.

45. The hybrid molecule of claim 44, wherein the linker is cleavable by a proteolytic enzyme.

46. The hybrid molecule of claim 44, wherein the linker is cleavable by a protease selected from the group consisting of one or more matrix metalloproteases, blood coagulation factors, neutrophil elastase, prostate specific antigen, and one or more plasminogen activators.

47. A pharmaceutical composition for the treatment of thromboembolism, the composition comprising the hybrid molecule of claim 1 and at least one pharmaceutically acceptable ingredient.

48. A pharmaceutical composition for the treatment of an infection, the composition comprising the hybrid molecule of claim 1 and at least one pharmaceutically acceptable ingredient.

49. A pharmaceutical composition for the treatment of cancer, the composition comprising the hybrid molecule of claim 1 and at least one pharmaceutically acceptable ingredient.

50. A pharmaceutical composition of the treatment of thromboembolic disease, the composition comprising the hybrid molecule of claim 1 and at least one pharmaceutically acceptable ingredient.

51. The pharmaceutical composition of claims 47-50, wherein the at least one pharmaceutically acceptable ingredient selected from the group consisting of solubilizing agents, buffers, vehicles, preservatives, local anesthetic, flavorings, colorings, stabilizers, and excipients.

52. A method for treating thromboembolism in a mammal, the method comprising administering an amount of a composition to the mammal effective to treat thrombus formation within the vasculature of said mammal, wherein the composition comprises the hybrid molecule of claim 1, and wherein the bioactive moiety is an anticoagulant.

53. A method for treating thromboembolism in a mammal, the method comprising administering an amount of a composition to the mammal effective to treat thrombus formation within the vasculature of said mammal, wherein the composition comprises the hybrid molecule of claim 1, and wherein the bioactive moiety is an antiplatelet agent.

54. A method for treating thromboembolism in a mammal, the method comprising administering an amount of a composition to the mammal effective to treat thrombus formation within the vasculature of said mammal, wherein the composition comprises the hybrid molecule of claim 1, and wherein the bioactive moiety is a thrombolytic.

55. A method of treating an infection in a mammal, the method comprising administering an amount of a composition to the mammal effective to treat the infection, wherein the composition comprises the hybrid molecule of claim 1, and wherein the bioactive moiety is an antibiotic.

56. A method of treating cancer in a mammal, the method comprising administering an amount of a composition to the mammal effective to treat the cancer, wherein the composition comprises the hybrid molecule of claim 1, and wherein the bioactive moiety is an antitumor agent.

57. The method of claim 52-56, wherein the composition is administered orally, or by intravenous or intraperitoneal subcutaneous injection.

58. The method of claim 52-56, wherein the mammal is a human.
